(19) United States
(12) Patent Application Publication
(10) Pub. No.: US 2026/0216516 A1
(43) Pub. Date: Jul. 30, 2026

(54) PATIENT ELIGIBILITY FOR AUTOMATIC INITIATION OF THERAPY BASED ON VARIABILITY OF SLEEP ONSET LATENCY

(71) Applicant: INSPIRE MEDICAL SYSTEMS, INC., Golden Valley, MN (US)

(72) Inventors: Maxwell P. Lundeen, Minneapolis, MN (US); Meghna Singh, Eden Prairie, MN (US); Kent Lee, Minneapolis, MN (US)

(73) Assignee: INSPIRE MEDICAL SYSTEMS, INC., Golden Valley, MN (US)

(21) Appl. No.: 19/143,469

(22) PCT Filed: Dec. 20, 2023
(86) PCT No.: PCT/US2023/085201

§ 371 Date: Jun. 26, 2025

Related U.S. Application Data
(60) Provisional application No. 63/477,710, filed on Dec. 29, 2022.

(51) Int. Cl. A61N 1/36

(52) U.S. Cl. CPC A61N 1/36139; A61N 1/3611

(57) ABSTRACT

A device and/or method to determine patient eligibility for automatic initiation of therapy based on variability of sleep onset latency. The device comprises a control portion programmed to determine patient eligibility of a patient, based on at least one variability metric of sleep onset latency of the patient, for initiating sleep disordered breathing (SDB) stimulation therapy via an automatic sleep detection mode. The automatic sleep detection mode may be a selection from a plurality of modes, wherein the plurality of modes may include a motion-activity only detection mode, a non-motion-activity physiologic parameter detection mode, a combination motion-activity and non-motion-activity physiologic parameter detection mode and a posture timer detection mode.

### BACKGROUND

A significant portion of the population suffers from various forms of sleep-related issues, some of which may involve sleep disordered breathing (SDB) and/or other conditions. Therapies for these sleep-related issues may involve sleep detection.

### BRIEF DESCRIPTION OF THE DRAWINGS

FIG. 1A is a diagram schematically representing an example method of determining sleep detection eligibility based on sensed physiologic information.

FIG. 1B is a diagram including a front view schematically representing a patient's body, implantable components, and/or external elements of example methods and/or example devices.

FIG. 1C is a schematic diagram of a control portion.

FIGS. 2-7 are diagrams schematically representing example methods and aspects of determining variability metrics of sleep onset latency of a patient.

FIG. 8 is a graph illustrating sleep onset latency distributions over a historical period of nightly treatment periods for a plurality of patients according to an example of the disclosure.

FIGS. 9-12 are diagrams schematically representing example methods of evaluating outliers in the sleep onset latency distributions of FIG. 8, for example.

FIG. 13 is a diagram schematically representing an example method in which sleep disordered breathing stimulation therapy is delayed.

FIG. 14 is a diagram schematically representing an example method in which multiple sensors are utilized to initiate therapy.

FIGS. 15-18 are a diagrams schematically representing an example method in which criteria is utilized to initiate therapy.

FIGS. 19-22 are diagrams schematically representing example methods comprising a plurality of automatic sleep detection modes and various types of automatic sleep detection modes of the disclosure.

FIGS. 23-24 are diagrams schematically relating to parameters or variability metrics for basing the at least one sleep onset latency variability metric according to examples of the disclosure.

FIGS. 25-26 are diagrams schematically representing example methods in which automatic sleep detection mode is initiated based on comparison to a threshold.

FIG. 27 is a diagram schematically representing an example method including receiving a patient input to initiated stimulation therapy.

FIG. 28 is a diagram schematically representing an example method including implementing the stimulation therapy to include electrically stimulating upper airway patency-related tissue of the patient.

FIGS. 29-31 are diagrams schematically representing example methods relating to timing of actuation of stimulation therapy.

FIG. 32 is a diagram schematically representing an example method including re-evaluating stimulation therapy protocol.

FIG. 33 is a diagram schematically representing criteria that can optionally be utilized in the example method of FIG. 32.

FIGS. 34-35 are diagrams schematically representing the example method of FIG. 32 further including receiving recorded sleep onset latency information and using that information to determine the sleep onset latency variability metric.

FIG. 36 is a diagram schematically representing the example method of FIG. 32 further including actuating the stimulation therapy after a set period of time after the patient is sensed to be in a sleep status.

FIG. 37 is a diagram schematically representing the example method of FIG. 32 further including the patient providing an input to indicate a nightly treatment period is about to begin prior to the step of applying stimulation therapy.

FIG. 38A is a diagram schematically representing an example timeline of sleep-wake-related events according to an example method of sleep-wake determination.

FIG. 38B is a flow diagram schematically representing an example method of determining a sleep-wake status.

FIG. 39 is a diagram schematically representing an example method of sensing physiologic information via sensing motion.

FIGS. 40A-40B are diagrams schematically representing an example method of determining a sleep-wake status relative to posture information.

FIG. 40C is a diagram schematically representing an example method of determining a sleep-wake status regarding different example sensed physiologic parameters.

FIG. 41A is a flow diagram schematically representing an example method of detecting sleep and/or maintaining stimulation therapy.

FIG. 41B is a diagram schematically representing an example method of detecting sleep.

FIG. 41C is a diagram schematically representing an example method including distinguishing body motion, posture, etc.

FIGS. 42-45 are diagrams schematically representing an example method of determining a sleep-wake status relative to respiratory phase information.

FIG. 46 is a flow diagram schematically representing an example method of determining a sleep-wake status regarding variability in physiologic signals/information.

FIGS. 47-48B are diagrams schematically representing an example method of determining a sleep-wake status relative to example motion information.

FIGS. 49A-49B are diagrams schematically representing an example method of determining a sleep-wake status via identifying variability in sensed physiologic information relative to a threshold.

FIGS. 50 and 51 are diagrams schematically representing an example method of determining a sleep-wake status via tracking parameters relating to time, activity, non-movement parameters, etc.

FIG. 52 is a diagram schematically representing an example method of determining a sleep-wake status according to a probability of sleep and/or a probability of wakefulness.

FIG. 53A is a diagram schematically representing an example method of determining a sleep-wake status regarding taking an action based on a probability of sleep and/or a probability of wakefulness.

FIGS. 53B-53H are diagrams schematically representing an example taking an action, in relation to a method of determining a sleep-wake status, including initiating or terminating stimulation, relative to various example boundaries regarding time, temperature, sleep stages, etc.

FIG. 53I is a diagram schematically representing an example method of receiving inputs regarding some of the example boundaries represented in at least FIGS. 53B-53H.

FIG. 54 is a diagram schematically representing an example method of determining a sleep-wake status including dividing sense signal(s) to enable assessing different sleep-wake determination parameters.

FIG. 55 is a diagram schematically representing an example method of determining a sleep-wake status according to a probability of sleep and/or a probability of wakefulness.

FIGS. 56 and 57 are diagrams schematically representing an example method of determining a sleep-wake status according to wakefulness information and snoring information, respectively.

FIG. 58 is a block diagram schematically representing an example sensing portion of an example device and/or used as part of example method for determining a sleep-wake status.

FIG. 59 is a block diagram schematically representing an example processing portion, which may form part of and/or be in communication with the example sensing portion.

FIG. 60A is a diagram including a front view schematically representing a patient's body and example implanted medical device for treating sleep disordered breathing and/or determining a sleep-wake status according to an example of the disclosure.

FIG. 60B is a diagram including a front view schematically representing an example implantable medical device with sensors.

FIGS. 60C and 60F are diagrams schematically representing different example implementations of example implantable medical devices as a microstimulator implanted in a head-and-neck region.

FIGS. 60D, 60E and 60G are schematic diagrams of example devices.

FIG. 61A is a block diagram schematically representing an example care engine.

FIG. 61B is a diagram schematically representing an example respiratory pattern.

FIGS. 62A-62B are block diagrams schematically representing an example control portions.

FIG. 63 is a block diagram schematically representing an example user interface.

FIG. 64 is a block diagram schematically representing example communication arrangements between a medical device and other devices, which may or may not comprise medical devices.

FIG. 65A is a diagram schematically representing an example user interface including example therapy usage patterns, sleep-wake status, sleep quality portions, use metrics, etc., which may be used in association with example method and/or example device for determining sleep-wake status.

FIGS. 65B-65C are diagrams schematically representing an example methods including taking an action in relation to a sleep-wake status determination.

FIG. 65D is a diagram schematically representing an example method of receiving input in relation to starting and/or stopping therapeutic treatment.

FIG. 65E is a diagram schematically representing an example method including tracking information regarding usage, starting, stopping, etc. of a therapy.

FIG. 66 is a diagram schematically representing an example method and/or example device including a medical device in relation to a resource for determining a sleep-wake status and/or sleep onset latency information (e.g. variability, etc.), including training and/or constructing a data model (e.g. machine learning model, heuristic model, etc.).

FIG. 67 is a diagram schematically representing an example method and/or example device for training and/or constructing a data model regarding determining a sleep-wake status (such as for sleep onset, latency, etc.).

FIG. 68 is a diagram schematically representing an example method and/or example device for determining a sleep-wake status (such as for sleep onset, latency, etc.) according to a trained and/or constructed data model.

FIG. 69 is an example chart for associating a patient with a cluster of similar patients.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

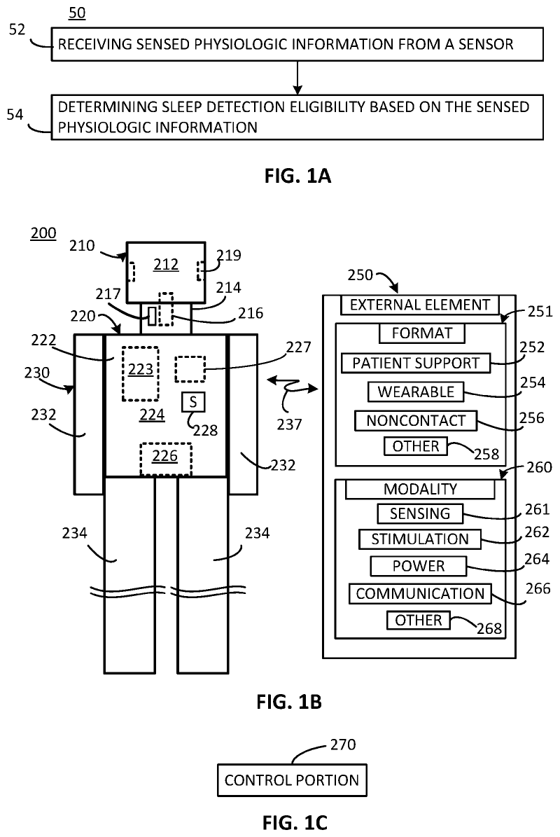

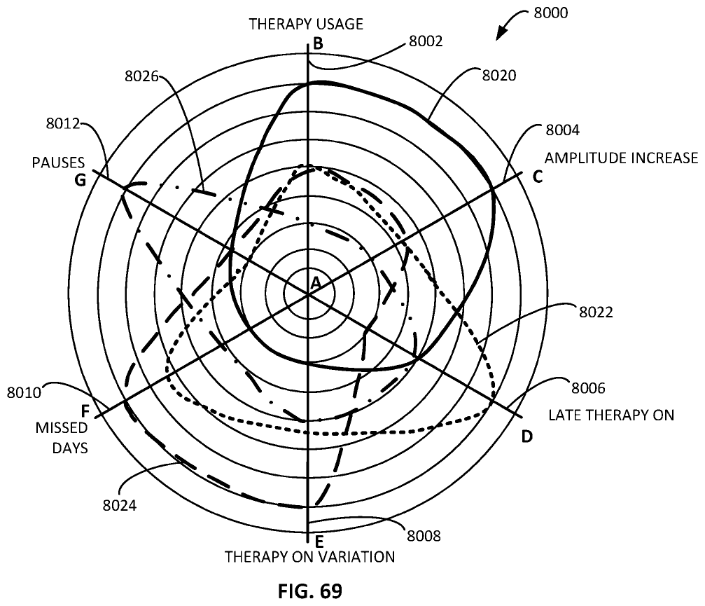

At least some examples of the present disclosure are directed to devices for diagnosis, therapy, and/or other care of medical conditions. At least some examples may comprise implantable devices and/or methods comprising use of implantable devices. However, in some examples, the methods and/or devices may comprise at least some external components. In some examples, a medical device may comprise a combination of implantable components and external components. At least some further examples of these arrangements are further described below in association with at least FIGS. 1A-69.

At least some of the example devices and/or example methods may relate to detecting at least sleep onset and related variability, the results of which may be used in caring for a patient such as (but not limited to) diagnosing, evaluating, monitoring, and/or treating a wide variety of patient conditions. In some such examples, such detection may be used to determine patient eligibility for automatic initiation of therapy such as (but not limited to) stimulation therapy.

At least some of the example devices and/or example methods, which may comprise monitoring, diagnosis, evaluation, and/or treatment (e.g. stimulation therapy). In some examples, the methods and/or devices may comprise automatically determining a sleep-wake status, which may in turn comprise detecting sleep and/or detecting wakefulness. In some examples, detecting sleep comprises detecting an onset of sleep. In some such examples, the sleep-wake determination may be used to initiate (and/or maintain) a treatment period in which neurostimulation therapy and/or therapy is delivered. In some of these examples, sleep-wake determination may comprise determining sleep onset latency, including sleep onset latency variability. In some examples, at least one variability metric of sleep onset latency may be used to determine patient eligibility for initiating therapy via automatic sleep detection. In one aspect, such automatic detection of sleep onset, which may be used to initiate therapy within a treatment period, is distinct from mere sleep staging for diagnostic purposes.

In some examples, the example devices and/or example methods may relate to sleep disordered breathing (SDB) care, which in some examples may comprise treating sleep disordered breathing via stimulation therapy and/or other therapy modalities.

Different patients have different sleep habits, whether those patients exhibit sleep disordered breathing and/or other conditions. For example, some patients that go to sleep at inconsistent times and take a long time to fall asleep on some nights but fall asleep right away after laying down on other nights. There are other patients, however, that go to sleep at consistent times and fall asleep quickly almost every night. For the patients that have inconsistent sleep habits, a sleep detection protocol may have a more difficult time detecting sleep onset latency (SL) and/or Wake After Sleep Onset (WASO). In some examples, such a protocol may be most accurate on patients with the most consistent sleep habits. Therefore, some example methods configured to initiate stimulation therapy for patients having inconsistent sleep habits may be programmed to accept a certain level of error for such patients. This accepted level of error can be reduced if the patient is screened to determine if their particular sleep habits meet criteria or thresholds for consistency. In some examples, certain aspects of the disclosure activate or initiate a protocol having a reduced level of accepted error for those patients.

In contrast to methods where patients with consistent (i.e. low variability) sleep habits become eligible for the sleep detection protocol, in alternate examples, the eligibility for the protocol could be reversed such that it is initiated for inconsistent sleepers but not consistent sleepers. The patients most benefiting from an automatic sleep detection protocol could be considered to be the ones with irregular sleep habits since a fixed therapy start delay timer alternative would likely not be sufficient for them to determine a start time for stimulation therapy. In this example, the protocol could be used in conjunction with the delay timer, wherein, if at the end of the timer, the protocol detects enough movement to classify the current state of the patient as awake, then an additional timer is added (mimicking the patient pressing a pause button on a patient therapy control remote). The method can also be used standalone, wherein stimulation therapy is turned on when the protocol has detected sleep with sufficiently high confidence to activate stimulation therapy. Therefore, the decision whether to apply the automatic sleep detection method may be made dependent on the quality of sensors available and the confidence placed in such sensors for a particular patient or patient group having certain characteristics.

At least some examples of determining sleep onset detection (including patient eligibility) also may relate to cardiac care, drug delivery, pelvic-related care, and/or other forms of care, whether standing alone or in association with sleep disordered breathing (SDB) care.

These examples, and additional examples, are further described in association with at least FIGS. 1A-69.

As schematically represented at 50 in FIG. 1A, in some examples a method comprises sensing physiologic information via at least one sensor 52, and determining a sleep detection eligibility via the sensed physiologic information 54. If such eligibility is established, sleep detection may be used to automatically initiate care (e.g. neurostimulation therapy) such as (but not limited to) SDB care. In some examples, determining sleep detection may comprise determining a sleep-wake status. Conversely, determining the sleep-wake status may comprise wakefulness detection by which the treatment period for care (e.g. SDB care) may be terminated automatically. In some examples in which a brief awakening is detected (versus prolonged wakefulness) and after which sleep is expected to be resumed, the treatment period is not terminated. Rather, the brief awakening may be deemed as a pause in the treatment period. Some example methods may comprise a time-based threshold (which may be just one factor of multiple factors) to determine whether the duration of wakefulness comprises a brief awakening or prolonged wakefulness.

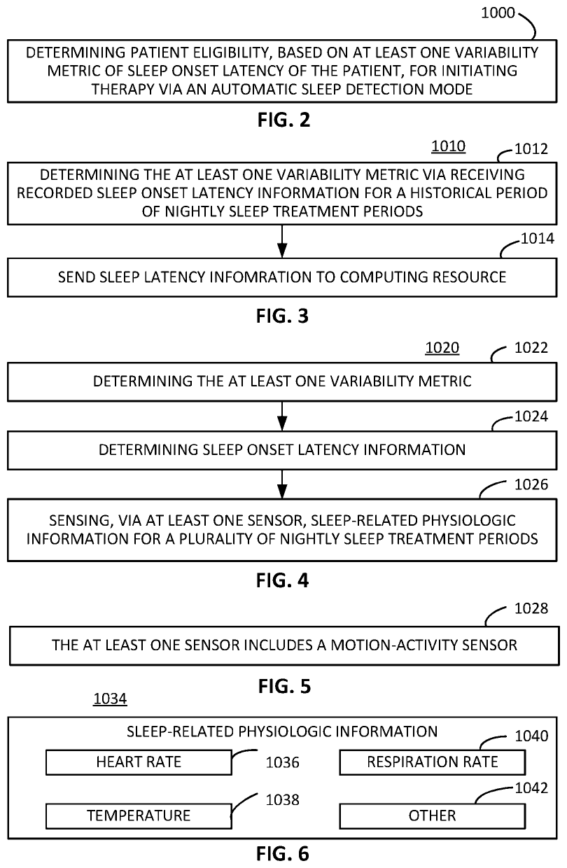

As further described below in association with at least FIGS. 2-68, in some examples determining a sleep-wake status may be associated with and/or form part of a method of determining patient eligibility for automatic sleep detection to initiate stimulation therapy (and/or other forms of therapy) in which such patient eligibility is based on at least one variability metric of sleep onset latency for the particular patient.

FIG. 1B is block diagram schematically representing a patient's body 200, including example target portions 210-234 at which at least some example sensing element(s) and/or stimulation elements may be employed to implement at least some examples of the present disclosure.

As shown in FIG. 1B, patient's body 200 comprises a head-and-neck portion 210, including head 212 and neck 214. Head 212 comprises cranial tissue, nerves, etc., and upper airway 216 (e.g. nerves, muscles, tissues), etc. As further shown in FIG. 1B, the patient's body 200 comprises a torso 220, which comprises various organs, muscles, nerves, other tissues, such as but not limited to those in pectoral region 222 (e.g. lungs 226, cardiac 227), abdomen 224, and/or pelvic region 226 (e.g. urinary/bladder, anal, reproductive, etc.). As further shown in FIG. 1B, the patient's body 200 comprises limbs 230, such as arms 232 and legs 234.

It will be understood that various sensing elements (and/or stimulation elements) as described throughout the various examples of the present disclosure may be deployed within the various regions of the patient's body 200 in order to sense and/or otherwise diagnose, monitor, treat various physiologic conditions such as, but not limited to those examples described below in association with FIGS. 2-68. In some such examples, a stimulation element 217 may be located in or near the upper airway 216 for treating sleep disordered breathing and/or a sensing element 228 may be located anywhere within the neck 214 and/or torso 220 (or other body regions) to sense physiologic information for providing SDB care including, but not limited to, sleep onset detection and related parameters.

In some examples, at least a portion of the stimulation element 217 may comprise part of an implantable component/device, such as an implantable pulse generator (IPG) whether full sized or sized as a microstimulator. The implantable components (e.g. IPG, other) may comprise a stimulation/control circuit, a power supply (e.g. non-rechargeable, rechargeable), communication elements, and/or other components. In some examples, the stimulation element 217 also may comprise a stimulation electrode and/or stimulation lead connected to the implantable pulse generator.

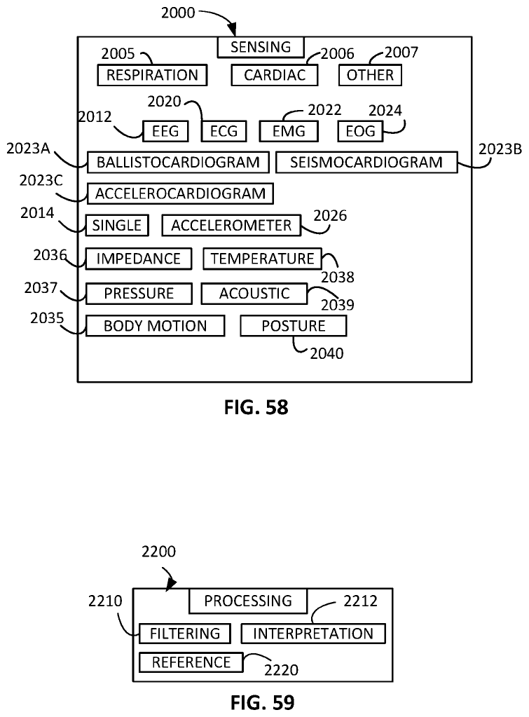

Further details regarding a location, structure, operation, and/or use of the sensing element 228, external element(s) 250, and/or stimulation element 217 are described below in association with at least FIGS. 1C-68, and in particular, at least FIGS. 58-61A.

In some examples, at least a portion of the stimulation element 217 may comprise part of an external component/device such as, but not limited to, the external component comprising a pulse generator (e.g. stimulation/control circuitry), power supply (e.g. rechargeable, non-rechargeable), and/other components. In some examples, a portion of the stimulation element 217 may be implantable and a portion of the stimulation element 217 may be external to the patient.

Accordingly, as further shown in FIG. 1B, the various sensing element(s) 228 and/or stimulation element(s) 217 implanted in the patient's body may be in wireless communication (e.g. connection 237) with at least one external element 250.

As further shown in FIG. 1B, in some examples, the external element(s) 250 may be implemented via a wide variety of formats such as, but not limited to, at least one of the formats 251 including a patient support 252 (e.g. bed, chair, sleep mat, other), wearable elements 254 (e.g. finger, wrist, head, neck, shirt), noncontact elements 256 (e.g. watch, camera, mobile device, other), and/or other elements 258.

As further shown in FIG. 1B, in some examples, the external element(s) 250 may comprise one or more different modalities 260 such as (but not limited to) a sensing portion 261, stimulation portion 262, power portion 264, communication portion 266, and/or other portion 268. The different portions 261, 262, 264, 266, 268 may be combined into a single physical structure (e.g. package, arrangement, assembly), may be implemented in multiple different physical structures, and/or with just some of the different portions 261, 262, 264, 266, 268 combined together in a single physical structure.

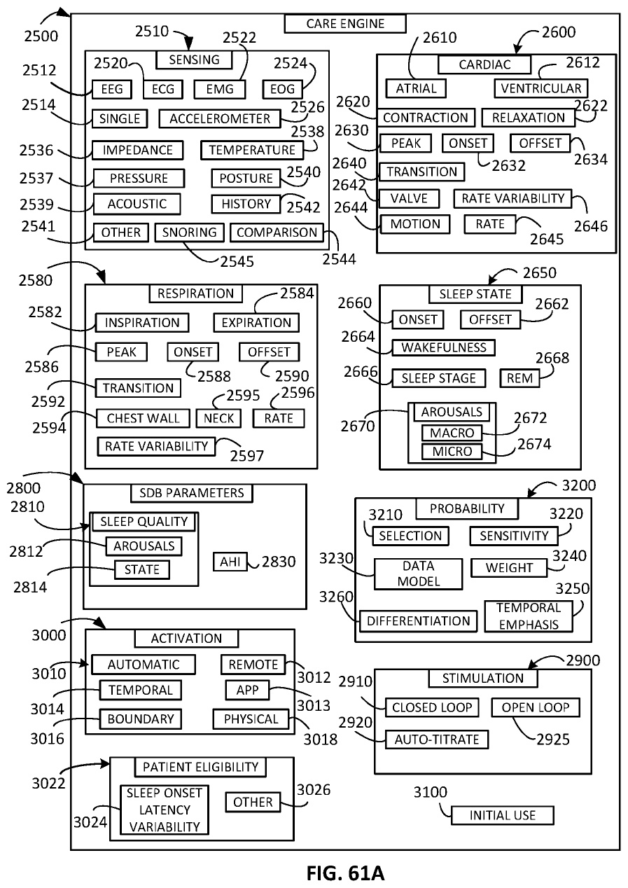

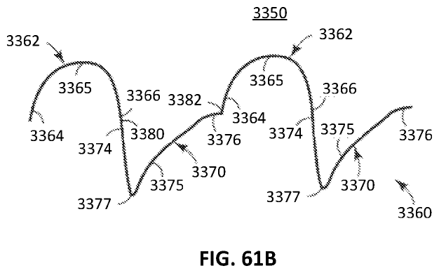

Among other such details, in some examples the external sensing portion 261 and/or implanted sensing element 228 may comprise an example implementation of, and/or at least some of substantially the same features and attributes of at least sensing portion 2000 and/or care engine 2500, as further described below in association with at least FIGS. 58 and 61A, respectively.

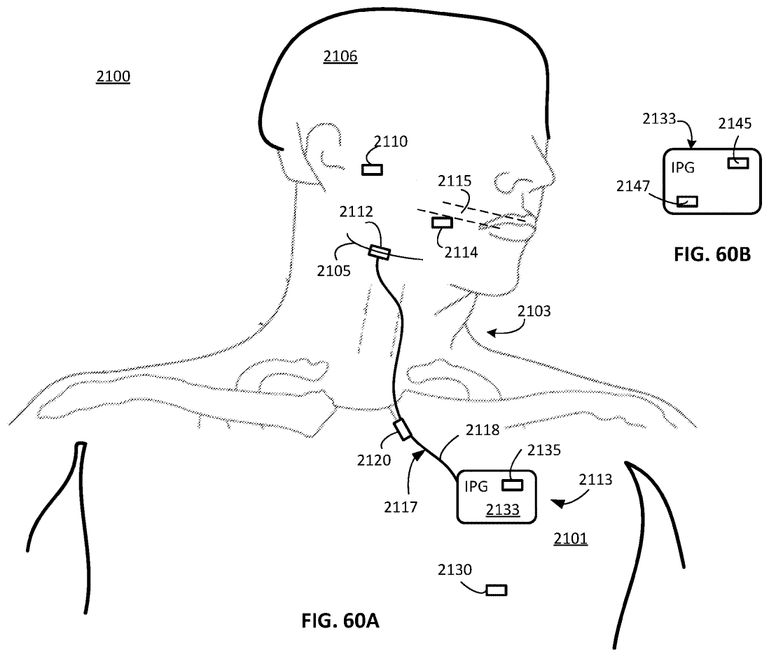

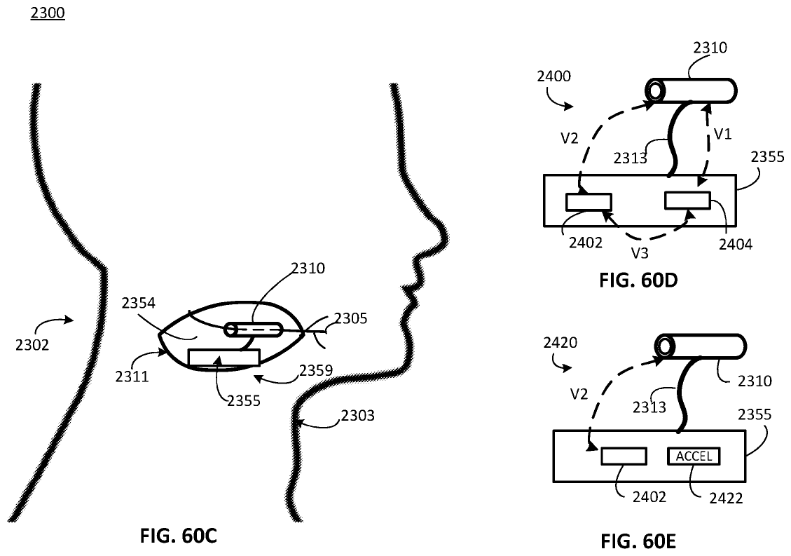

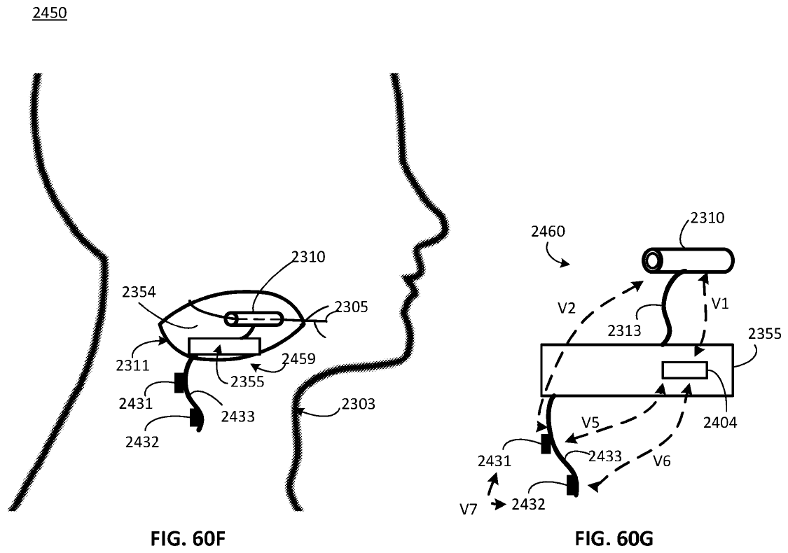

In some examples, the stimulation portion 262 and/or implanted stimulation element 217 may comprise an example implementation of, and/or at least some of substantially the same features and attributes as, at least the stimulation arrangements as further described below in association with at least FIGS. 60A-60G and/or other examples throughout the present disclosure.

In some examples, the external power portion 264 and/or power components associated with implanted stimulation element 217 may comprise at least some of substantially the same features and attributes of at least the stimulation arrangements, as further described below in association with at least FIGS. 60A-60G and/or other examples throughout the present disclosure. In some such examples, the respective power portion, components, etc. may comprise a rechargeable power element (e.g. supply, battery, circuitry elements) and/or non-rechargeable power elements (e.g. battery). In some examples, the external power portion 264 may comprise a power source by which a power component of the implanted stimulation element 217 may be recharged.

In some examples, the wireless communication portion 266 (e.g. including connection/link at 237) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 268 via which other aspects of implementing medical care may be embodied in external element(s) 250 to relate to the various implanted and/or external components described above.

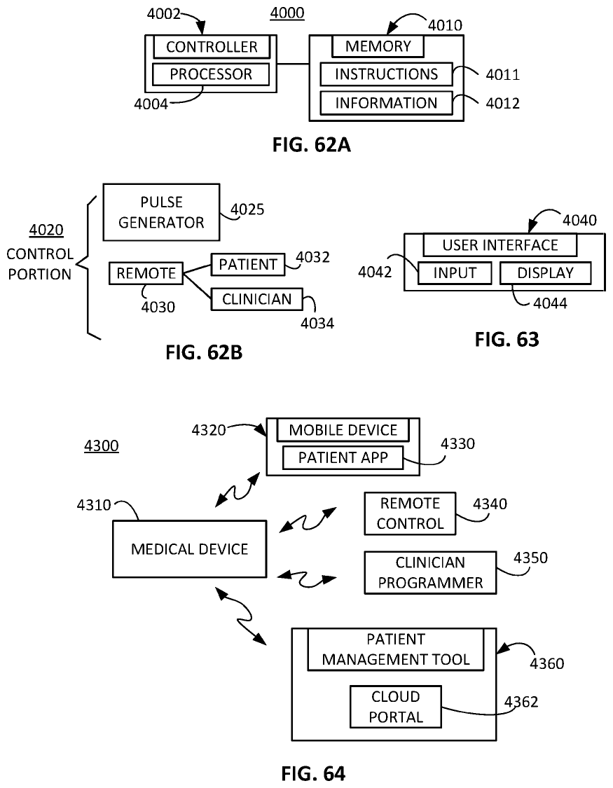

FIG. 1C schematically represents a control portion 270, which may comprise at least some of substantially the same features and attributes as the control portion 4000 in FIGS. 62A-64 and/or care engine 2500 in FIG. 61A. In some examples, the control portion 270 will be part of a care engine (e.g. 2500 in FIG. 61A) or the like. Among other aspects, example methods and/or example devices may be implemented via the control portion 270. In some examples, the control portion 270 may be used to implement at least some of the various example devices and/or example methods of the present disclosure as described herein. In some examples, the control portion 270 may form part of, and/or be in communication with, the stimulation element (e.g. 217 in FIG. 1B) or other medical devices (or portions thereof), as further described later.

In various examples, the control portion 270 is programmed to determine patient eligibility of a patient based on at least one variability metric of sleep onset latency of the patient for automatic sleep detection mode. In some examples, at least some aspects of this example arrangement may be implemented via a patient eligibility portion 3022 of care engine 2500 in FIG. 61A, as further described later.

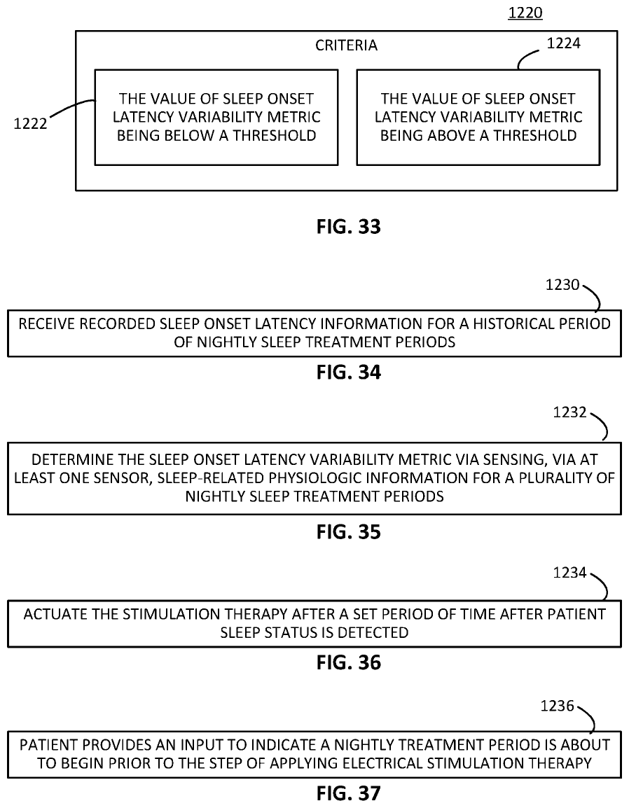

The at least one variability metric of sleep onset latency of the patient and automatic sleep detection modes will be discussed in greater detail below. In some examples, the variability metric of sleep onset latency of the patient is dependent on at least one of a measure of time and a measure of a physical parameter of the patient. In various examples, the control portion 270 is programmed to initiate a standard initiation mode upon the at least one variability metric of sleep onset latency of the patient not meeting criteria. Aspects regarding standard initiation modes of the disclosure are discussed in greater detail below with respect to at least FIG. 33.

In some examples, the automatic sleep detection mode may, in turn, be used as part of automatically initiating a therapy, e.g. a stimulation therapy. In some examples, the stimulation therapy may comprise a therapy to treat sleep disordered breathing (SDB).

Referring in addition to FIG. 2, various methods of the disclosure include determining patient eligibility based on at least one variability metric or score of sleep onset latency of the patient for an automatic sleep detection mode 1000. Such methods can be achieved at least in part with the control portion 270 of FIG. 1D, for example.

Referring now also to FIG. 3, in some examples, a method 1010 includes the step of determining the at least one variability metric via receiving recorded sleep onset latency information for a historical period of nightly sleep treatment periods 1012. Optionally, the sleep onset latency information can be sent to a computing resource 1014 for one or more of storage and processing. In some examples, the historical period of nightly sleep treatment periods includes a selectable number of multiple nights such as, but not limited to, two or more nights, three or more nights, seven or more nights, fourteen or more nights, thirty or more nights, sixty or more nights, or ninety or more nights.

Referring now in addition to FIG. 4, in some example methods 1020, the step of determining the at least one variability metric of sleep onset latency of the patient 1022 is achieved at least in part by determining sleep onset latency information 1024, which is achieved by sensing, via at least one sensor, sleep-related physiologic information (e.g. parameter(s)) for a plurality of nightly sleep treatment periods 1026. As shown in FIG. 5, one sleep-related physiologic parameter may comprise, in some non-limiting examples, a motion-activity parameter which may be sensed via a motion-activity sensor 1028, which may comprise an accelerometer or other type of motion-detecting sensor. The accelerometer may comprise a tri-axis accelerometer in one example. As shown in FIG. 6, other sleep-related physiologic information 1034 can include one or more of a heart rate of the patient 1036, temperature of the patient 1038, respiration rate of the patient 1040, among other parameters 1042 for example. It will be understood that in some examples, an accelerometer may be used to sense the other sleep-related physiologic information such as, but not limited to, the heart rate, respiration rate, etc. This accelerometer may be the same or different from an accelerometer used to sense motion-activity. In some examples, a temperature sensor is used to sense the temperature of the patient, wherein the temperature sensor may comprise an internal sensor (e.g. incorporated within an IPG) and/or external temperature sensor (e.g. bed mat, nasal airflow, etc.).

Other optional sleep-related physiologic information (e.g. parameters) which can be sensed are later described in association with at least sensing portion 2000 of FIG. 58 and/or in association with the care engine 2500 of FIG. 61A, for example. The aforementioned examples of sleep-related physiologic information (e.g. parameters) are not intended to be an exhaustive or limiting list but are provided as examples of such physiological information.

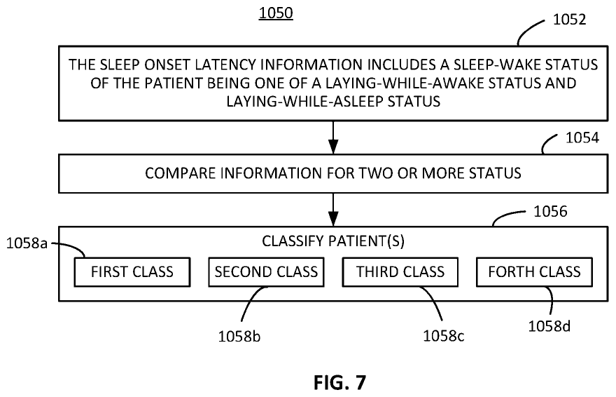

Referring now in addition to FIG. 7, in some example methods 1050 the sleep onset latency information can include a sleep-wake status or state of the patient 1052. Among other aspects, in some examples the sleep-wake status can include one of a laying-while-awake status indicating that the patient is not yet asleep but is in a sleep position (e.g. laying horizontally, supine, reclined, prone, left decubitis, right decubitis or other sleep associated position). The sleep-wake status can also include a laying-while-asleep state indicating that the patient is asleep and is laying down in any aforementioned sleep position. The present disclosure is not intended to be limited solely to these two examples of comparing sleep-wake statuses as some patients may sleep in positions other than a traditional sleep positions.

At 1054, some example methods (e.g. method 1050) may include comparing sleep-related physiologic information between a sleep status or state and a wake status or state. The comparison may be used to gauge whether there is a significant difference in one or more sleep-related physiologic parameters of a patient while they are laying-while-awake versus laying-while-asleep. Various examples include organizing or classifying or grouping one or more patients in classes or groups based one or more characteristics 1056. In one example, for a first class 1058a of patients their motion-activity in a wake state is significantly different from their motion-activity in a sleep state, with this relationship corresponding to these patients tending to have shorter and more consistent sleep onset. Accordingly, as further described later, a sleep-wake determination may be performed via sensing solely a motion-activity parameter. However, for other classes of patients, their motion-activity (between an awake state and a sleep state) is not significantly different enough to rely solely on sensing a motion-activity parameter in order to determine a sleep-wake status. In some such examples, sensing one or more other physiologic parameters (e.g. 1034 in FIG. 6) may be used to determine a sleep-wake status, whether in addition to (or instead of) sensing motion-activity.

For example, for a second class 1058b of patients, their motion-activity while in a wake state is at least somewhat different from their motion-activity when in a sleep state, and other sensed physiologic parameters (e.g. 1034 in FIG. 6) are different enough (between an awake state and a sleep state) to assist in sleep-wake determination. This second class 1058b of patients may have some problems falling asleep on occasion but otherwise fall asleep regularly without too many exceptions. In some examples, such exceptions may be referred to as outliers, as described later more fully below in continued reference to FIG. 8.

In some examples, a third class 1058c of patients may exhibit motion-activity in a wake state which is at least somewhat different from their motion-activity in a sleep state, and other sensed physiologic parameters (e.g. 1034 in FIG. 6) are different enough (between an awake state and a sleep state) to assist in making a sleep-wake status determination. However, this third class 1058c of patients may have frequent problems falling asleep such that their sleep onset latency is much larger and much more variable (than the sleep onset latency of the first and second classes of patients), with the third class 1058c of patients exhibiting some large outliers in their sleep onset latency distribution (e.g. in some instances it takes an hour or more to fall asleep).

In some examples, for a fourth class 1058d of patients, most of the sensed physiologic parameters (e.g. 1028 in FIGS. 5 and 1034 in FIG. 6) for their wake state do not differ enough from their sleep state in order to use the sensed physiologic parameters to make a sleep-wake determination. Among other aspects, these patients may exhibit a large variation in their sleep onset latency and can have extreme outliers (e.g. in some instances it takes two hours or more to fall asleep). In some such examples, a timer, other sensed physiologic parameters (e.g. posture), and/or other patient inputs may be used to make a sleep onset determination, as further described later.

Therefore, in some examples, at least some of the above-noted comparisons of values of physiologic parameters and/or of the associated different classes of patients can be used in the application of or selection of an automatic sleep detection mode. In some such examples, this arrangement may be further understood in association with examples of multiple automatic sleep detection modes as described later in association with FIG. 20 and related disclosure.

It will be understood that patients may be grouped into a greater number or lesser number of classes than the example of classes described above.

Some example methods may comprise comparing a value of at least one sensed sleep-related physiologic parameter exhibited by a patient for their wake status versus their sleep status. If there is a great enough difference in the sensed physiologic parameter of the patient between their respective sleep and wake statuses, the example method includes authorizing implementation of an automatic sleep detection mode. The automatic sleep detection mode is configured to initiate therapy when the sensed value of the physiologic parameter (e.g. motion-activity or other physiological parameter) of the patient is indicative of a sleep status. In some examples, the comparison is made while the patient is laying down such that the comparison is between a laying-down-while asleep status and a laying-down-while awake status, which can be signaled by the patient via a patient remote or the like, for example. In some such examples, the initiated therapy may comprise therapy such as (but not limited to) SDB therapy, which may comprise stimulation therapy and/or other forms of therapy.

In some examples, the comparison is made with reference to a criterion or criteria. In such examples, the automatic sleep detection mode will be authorized if the above-mentioned difference is large enough to meet the criterion, i.e. there is a large enough difference between the sensed motion-activity when asleep versus the sensed motion-activity when awake in order to easily determine sleep-wake status solely via the motion-activity parameter. Stated differently, the patient becomes eligible for initiation of therapy (e.g. SDB therapy) via an automatic sleep detection mode when the difference (between sensed motion-activity when asleep and sensed motion-activity when awake) meets a criterion.

In some examples, the determination of sleep-wake status may be performed without sensing body position (e.g. posture). However, in some examples, the determination of sleep-wake status may be performed using sensed body position (e.g. posture), as further described later in association with at least FIG. 20. In some such examples, sensing body position may be performed via an accelerometer such as, but not limited to, a tri-axis accelerometer.

In some examples, the therapy (e.g. to be initiated via an automatic sleep detection mode) may be initiated in association with a delay timer. In some such examples, a magnitude of the time delay may be based on and/or associated with a magnitude of difference between sensed physiologic parameter(s) of a sleep state and sensed physiologic parameter(s) of an awake state. In some of these examples, the magnitude of difference between the sensed physiologic parameters (of a respective sleep state and an awake state) may fall within different zones. For instance, there may be a first zone corresponding to a first range of magnitude difference and for which the time delay may comprise a first value (e.g. 30 minutes) and there may be a different second zone corresponding to a second range of magnitude of difference for which the time delay may comprise a different second value (e.g. 40 minutes).

However, in some examples, the different second value of time delay may be less than the first value of time delay. Moreover, in some examples, there may be a greater number or a fewer number of zones. In various examples, the first and second zones do not overlap. In other examples, the first and second zones overlap.

Generally, for patients having statistically significant differences in their sensed physiologic parameters information (e.g. motion-activity in FIG. 5 and/or other parameters in FIG. 6) between their respective wake and sleep states, at least some aspects of the disclosure enable a relatively basic sleep detection protocol that looks for changes in these signals alone to enable therapy (e.g. stimulation therapy for SDB and/or other conditions). At least some example systems and/or methods of the disclosure can supplement the sleep detection protocol with various timers, checks, and balances for patients with minor differences in sensed patient physiologic parameters. Such methods may be configured to wait predetermined amounts of time before checking for sleep regardless of the patient's sensor signals relating to a wake-sleep status. In one example, the predetermined amount of time to wait before checking patient signals indicating a wake-sleep status is variable based on the patient's sleep history (see also, history 2542 of FIG. 61A). For patients with no noticeable statistically significant difference between their sensed physiologic parameter when a patient is in an awake status or sleep status, some example methods of the disclosure may include a delay timer that starts based on the detection of a sleep posture or position of the patient detected by one or more sensors and/or resets based on a change in posture of the patient. In this example, once the delay timer runs out, therapy may be initiated if no later change in posture is detected that may trigger a restarting to the delay timer due to the change in posture indicating that the patient is likely in the wake status and is not asleep.

Referring now in addition to FIG. 8, when sleep onset latency information is obtained, at least one variability metric of sleep onset latency of the patient can be evaluated. Generally, FIG. 8 is a graph 1060 of sleep onset latency distribution (minutes) for a plurality of patients illustrating how patients may be eligible for different method implementations, modalities corresponding to a designation (e.g. 1062, 1064, 1066, 1068) based on their respective eligibility scores. A first modality associated with a first designation 1062 could be a motion-activity detection mode (see also FIG. 20 and related disclosure). A second modality associated with a second designation 1064 could be a combination mode (see also FIG. 20 and related disclosure). A third modality associated with a third designation 1066 could be a combination mode with timer (see also FIG. 20 and related disclosure). A fourth modality associated with a fourth designation 1066 could be a posture-timer mode (see also FIG. 20 and related disclosure). In some examples, the system may choose the least burdensome or most practical sensing modality or option, as long as the patient's eligibility score and calibration period (as discussed below) allow it. In some examples, the fourth modality may employ the general principles of a posture-timer modality but utilize a physiologic information/parameter other than, or in addition to, posture in a complementary manner with a timer.

In one non-limiting example, the most practical sensed physiologic parameter from which to make a sleep-wake status determination may comprise a motion-activity parameter. As previously mentioned, in some examples the motion-activity parameter may be sensed solely via a motion-activity sensor, such as but not limited, to an accelerometer in some examples.

In the graph of FIG. 8, the plurality of patients are organized so that the patients to the left side of the graph have a shorter sleep onset latency distribution as compared to the patients to the right on the graph. From this information, patients can be categorized or grouped by recommended sensing modalities by which sleep onset may be detected, as will be discussed in greater detail below. In some examples, each mode will correspond to a zone or section on the graph along the x-axis 1061B. In various examples, the designations 1062, 1064, 1066, 1068, which may be considered to define various zones for designations on the graph of FIG. 8 can have some overlap.

Additional information regarding example modes of the disclosure can be found below with respect to FIGS. 20-22.

As shown in FIG. 8, the x-axis 1061B represents different patients and the y-axis 1061A represents a sleep onset latency distribution including a number of minutes for a patient to fall asleep since the patient has been in bed (i.e. for sleep onset to occur) over a plurality of different nights, such as a selectable number of different nights. It will be understood that the y-axis 1061A can be another measure of time in other examples.

As shown in FIG. 8, in some examples the sleep onset latency distribution for each patient may be expressed as a box-and-whisker plot. In one aspect, the box-and-whisker plot enables the cumulative data of a plurality of nights during which sleep onset latency was measured to be displayed in a manner which illustrates a degree of variability in sleep onset latency for a particular patient. However, it will be understood that the sleep onset latency distribution may be represented via tools other than a box-and-whisker plot.

As further shown in FIG. 8, for each patient, a box-and-whisker plot 1070 includes a box 1071A which extends between a first end 1071C and opposite second end 1071D, with plot 1070 including a median 1071B located between respective ends 1071C (upper 75% of data), 1071D (lower 25% of data). In other examples, the median 1071B may be a mean value or the like. The box 1071A represents about 50 percent of the sleep onset latency data for a patient. The length of the box 1071A may provide an indication of a degree of variability in sleep onset latency in which the longer the box 1071A, the greater degree of variability in sleep onset latency and the shorter the box 1071A, the lesser degree of variability in sleep onset latency.

Accordingly, for example, the box-and-whisker plot for the particular patient shown as 1070 in FIG. 8 includes a long box (e.g. 1071A) beginning at a lower end (e.g. 1071D) of about 30 minutes of sleep onset latency and extending up to top end (e.g. 1071C) about 100 minutes of sleep onset latency, which indicates a high degree of variability of sleep onset latency, where it commonly may take that patient anywhere from 30 minutes to 100 minutes for sleep onset (i.e. to fall asleep) to occur. In addition, the box-and-whisker plot 1070 includes whiskers 1072A, with a top whisker 1072A extending from top end 1071C of box 1071A to an outer end 1072B, and a bottom whisker 1072C extending from bottom end 1071D of box 1071A to an outer end 1072D. In one aspect, the respective whiskers 1072A, 1072C extend 1.5 times the range of box 1071A, such that each respective whisker 1072A, 1072C extends to a value that is furthest from the center while still being inside a distance of 1.5 time the interquartile range from the lower or upper quartile. Any data points falling outside the end of the respective whiskers (e.g. 1072A, 1072C) is classified as an outlier.

As shown in FIG. 8, the top whisker 1072A represents a large range of variability in sleep onset latency, falling within a range between 100 minutes and 175 minutes, while the bottom whisker 1072C represents a large range of variability in sleep onset latency, falling within a range between 0 minutes and 30 minutes of sleep onset latency. Considering the box-and-whisker plot 1070 for this particular patient as a whole, it is apparent that a wide range of sleep onset latency is exhibited, from 0 to 175 minutes. In addition, in some instances for this patient, a given night of sleep onset latency falls outside the entire box-and-whisker plot 1070, such as outlier 1073A at which it took about 215 minutes for the sleep onset to occur on one night. In some examples, other patients may exhibit multiple extreme outliers, such as shown at 1073B, which represents several different nights on which it took more than 200 minutes for sleep onset to occur, and at 1073C, which represents a couple nights on which it took more than 300 minutes for sleep onset to occur.

In sharp contrast, FIG. 8 also illustrates other example box-and-whisker plots such as, but not limited to, plot 1074 at the far left end of the graph 1060. For convenience and illustrative simplicity, similar reference numerals for box-and-whisker plot 1070 will be used to refer to the respective box, whiskers, outliers, etc. of box-and-whisker plot 1074. With this in mind, box-and-whisker plot 1074 includes a very short box 1071A (e.g. between 5 and 15 minutes), a relatively low median 1071B (e.g. 10 minutes) short upper whisker 1072A (e.g. between 10 and 25 minutes), short lower whisker 1072C (e.g. between 5 and 10 minutes), and a couple of close outliers 1073A at about 27 and about 35 minutes.

As can be further observed from the plurality of box-and-whisker plots shown in FIG. 8, a wide range of sleep onset latency variability is exhibited across the spectrum of patients between the left end of the graph (e.g. plot 1074) and the right end of the graph (e.g. plot 1070).

With this example patient population in mind, at least some examples of determining patient eligibility for automatic sleep detection (based on variability in sleep onset latency) may be further understood in context with Example 1:

Patient A was found to be a good candidate for obstructive sleep apnea therapy via an implantable pulse stimulation generator. In addition to physiologic conditions and behaviors associated with obstructive sleep apnea, Patient A has insomnia. Patient A is instructed to sleep on an external sensing sleep mat some amount of days (an “eligibility period”) before device implant/activation. In some examples, the external sensing may comprise a Withings® sleep mat available from www.withings.com, and headquartered in Issy-les-Moulineaux, France. However, it will be understood that sensing formats and/or modalities other than a sleep mat may be used in addition to, or instead of, the sleep mat, and that sleep mats/similar other than the Withings® sleep mat may be used.

In one aspect, the sleep mat non-invasively measures Patient A's sleep habits and senses sleep-related physiologic information during the eligibility period. On some nights, similar to what is shown in plot 1070, Patient A may struggle to fall asleep for hours due to their insomnia, and on other nights they fall asleep quickly due to being overtired.

In some examples, a sleep eligibility score for Patient A (and other patients) can be computed given the following:

After observing the patient's sleep habits over n days, the following parameters can be calculated:
1. Sleep onset latency for day i, sol(i)
2. Mean sleep onset latency, μₛₒₗ
3. Variability in sleep onset, σₛₒₗ
4. Number of outliers, nₒ (days when the sleep onset latency is more than two standard deviations away from the mean sleep onset latency)

$${n}_{o} = \# ( \vert \frac{sol ( i ) - {μ}_{sol}}{- {σ}_{sol}} \vert > 2 )$$

5. Number of pauses on therapy remote, if the measurements are taken post-activation, nₚ
   In some examples, the eligibility score, es can be computed as:

$$es = {w}_{1} {μ}_{s o l} + {w}_{2} {σ}_{s o l} + {w}_{1} {n}_{o} + {w}_{4} {n}_{p}$$

where w₁, w₂, w₃, w₄ are the weights assigned to each of the parameters. These weights can be determined by performing statistical analysis on the collected data.

In some examples, a threshold can be set on this eligibility score such that any patient whose eligibility score crosses the threshold becomes eligible for a sleep detection protocol (under or over the threshold, depending on whether the eligibility is for consistent sleepers or inconsistent sleepers respectively).

For example Patient A and/or other patients with problematic sleep behaviors/conditions, it is believed that at least some sleep detection protocols may not be accurate enough to predict these vast variabilities in sleep behavior. Accordingly, at a first point in time, Patient A may receive designation 1068 for which a posture timer sleep detection protocol is implemented, as being ineligible for one of the other sleep detection protocols (e.g. designations 1062, 1064, 1066), or as being ineligible for any sleep detection protocol. However, as time goes on, Patient A may receive therapy for their insomnia, and learn to manage their insomnia better. Patient A may continue to sleep on their external sensor Sleep Mat (and/or sleep with other sensor modalities, formats) for additional nights for an additional period of time, which may or may not be when the therapy modalities (e.g. pulse stimulation generator) is activated. During the additional period of time, the external sensor sleep mat measures their new and improved sleep habits (i.e. a reduced variability in sleep, which falls within designation 1062). Patient A's new eligibility scores are measured against the known eligibility criteria via a computing resource (e.g. in the cloud), and Patient A may now be eligible for the automatic sleep detection protocol implementation associated with the designation 1062. This automatic sleep detection protocol may be activated the following night, and Patient A may be informed that they are eligible for automatic sleep detection to activate stimulation therapy. Accordingly, Patient A generally would no longer need to use their Patient Remote Control to turn on, pause, and/or turn off their therapy device (e.g. pulse stimulation generator). However, as just one example, Patient A may pause or turn off their therapy device if Patient A judges that stimulation therapy was activated when it should not have been. In some examples, the notification of eligibility for automatic sleep detection may be communicated via a mobile phone application or other methods. The aforementioned Example 1 is not intended to be limiting of the disclosure.

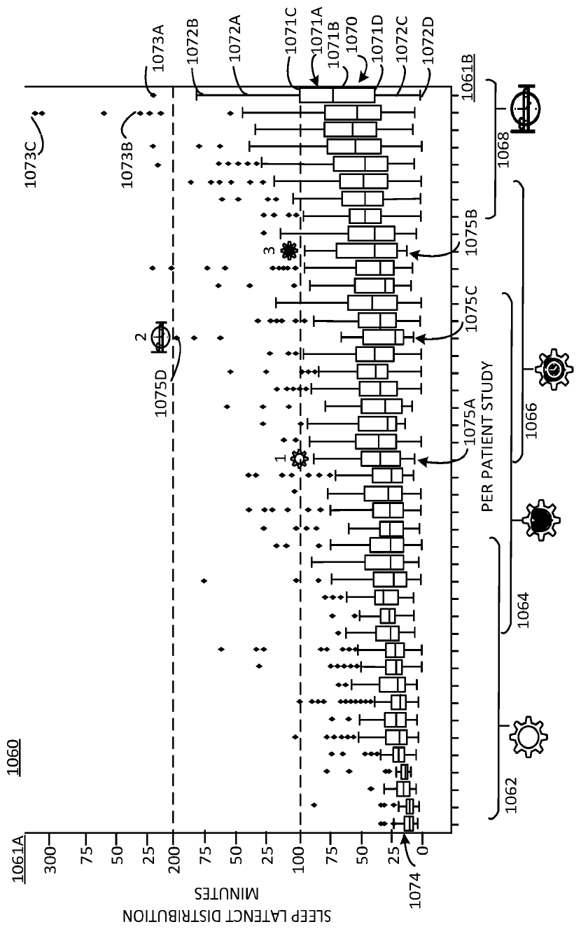

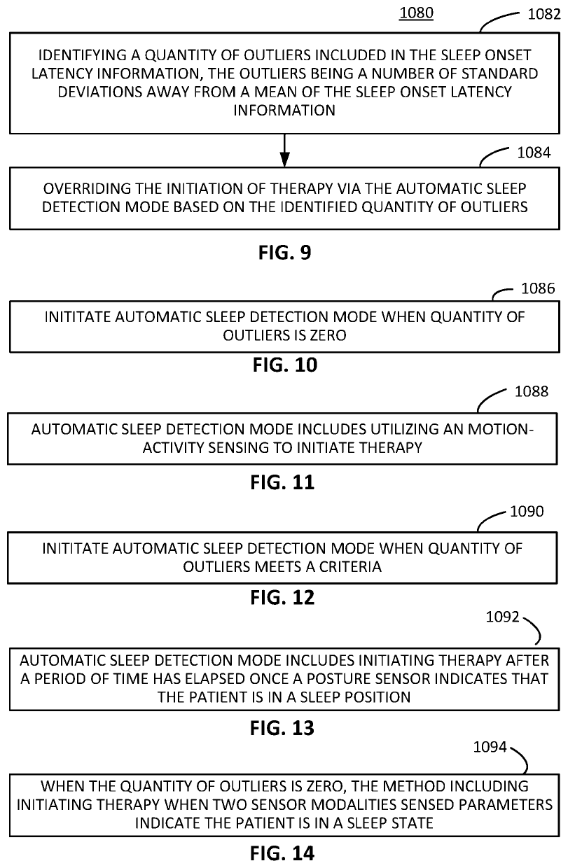

Referring now in addition to FIG. 9, in some example methods 1080, the sleep onset latency information includes outliers as noted above. Depending on a patient's quantity of outliers, severity of outliers, etc., which contribute to variability (or lack thereof) in sleep onset latency, certain patients may benefit from more strict sleep detection protocols even though their mean or median sleep onset latency is on the lower (i.e. left) end of the graph. Possible exceptions to those methods are possible in various example methods. In one example, Patient 1 (1075A) identified in FIG. 8 has no outliers, which indicates they could meet criteria be a candidate for a motion-activity only detection mode due to their relative lack of variation in sleep onset latency and relative lack of outliers in sleep onset latency. In one example, Patient 2 (1075C) identified in FIG. 8 has a relatively small sleep onset latency variability distribution but a few extreme outliers (e.g. 1075D at 200 minutes sleep onset), meeting criteria to make them a candidate for posture timer detection mode due to the unpredictability of their sleep patterns. Additionally, in another example Patient 3 (1075B) identified in FIG. 8 has a relatively large spread of sleep onset latency variability distribution but has no outliers, meeting criteria making them a suitable candidate for a combination sensing/detection modality mode (see also, FIG. 20 and related disclosure which discusses various combination detection modes, e.g. motion-activity only detection mode and other physiologic parameter detection mode.

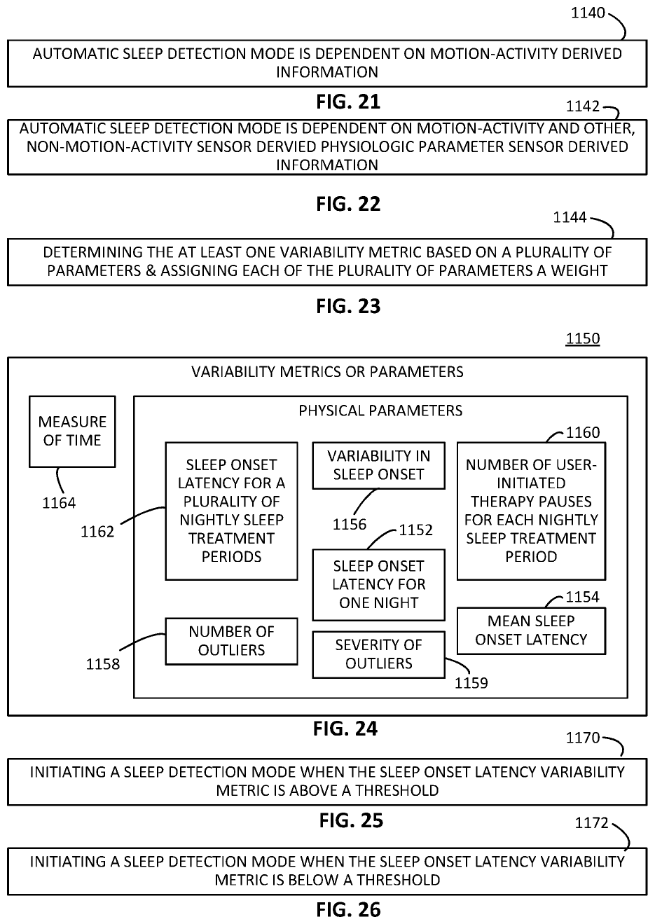

The relative severity of an outlier (e.g. 1075B) can be identified, in some examples, by the number of standard deviations by which the outlier (e.g. 1073B, 1073C, 1075D) is away from a mean of the sleep onset latency information 1082. Optionally, in some examples method 1080 can include overriding the initiation of therapy via the automatic sleep detection mode based on the identified quantity of outliers 1084. In one non-limiting example, the number of standard deviations for defining an outlier is two or more. Referring now to FIG. 10, once any outliers are identified, some example methods further include initiating automatic sleep detection mode when the quantity of outliers is zero 1086. In the example of FIGS. 10-11 for such a scenario with the number of outliers is zero, automatic sleep detection mode may be configured to utilize solely a motion/activity sensor (such as via an accelerometer) to initiate the therapy (e.g. SDB therapy), as shown at 1088. In some examples, the SDB therapy may comprise application of stimulation via stimulation element 217 (FIG. 1B) to an upper airway patency-related tissue, which may comprise a target nerve and/or target muscle. In some such examples, the target tissue/nerve may comprise a hypoglossal nerve, an infrahyoid-related nerve, other upper airway patency-related nerve, and/or muscles innervated by such example nerves. In some examples, the infrahyoid-related nerve may comprise an infrahyoid (IH) muscle-innervating nerve which innervates at least one of the infrahyoid strap muscles, such as, the sternohyoid, sternothyroid, omohyoid, and/or thyrohyoid. Such a mode may be the motion-activity only detection mode 1132 of FIGS. 20-21, further discussed below. As noted elsewhere, in some examples the motion-activity only detection mode may be implemented solely via an accelerometer (e.g. tri-axis accelerometer in some examples).

Referring in addition to FIG. 12, it is envisioned that it may be desirable to initiate automatic sleep detection mode when the quantity of outliers meets a criteria or falls within a prescribed zone (i.e. range) 1090. As indicated above, the criteria may be that the quantity of outliers is zero. In other examples, the criteria may be in a range that includes zero. In additional examples, the criteria may be in a range exclusive of zero outliers. In yet another example, the criteria may be that the quantity of outliers is more than a certain number or threshold. In an additional example, the criteria may be that the quantity of outliers is less than a certain number or threshold.

Referring in addition to FIG. 13, in various example methods, automatic sleep detection mode can include initiating the therapy (e.g. SDB stimulation therapy) after a period of time has elapsed during which one or more sensors (e.g. sensors 228, 250) indicate that the patient has not changed their body position (e.g. posture), which in turn may be indicative of the patient being in a sleep state/status as shown at 1092 in FIG. 13. In one illustrative example, the sensor is a posture (e.g. body position) sensor (e.g. see sensor 2540 of FIG. 61A and related disclosure). For example, the body position sensor may sense that the patient has remained in a lying down (e.g. horizontal) or sleep position for a selectable period of time. Examples of one “posture timer detection” mode is further discussed with respect to FIG. 20.

Referring in addition to FIG. 14, in one example when the quantity of outliers is zero, some example methods of the disclosure can include initiating SDB stimulation therapy (or other therapies) when at least two sensor modalities sensed physiologic parameters (which can be sensed with one or more of 228, 250 in examples of the disclosure) indicate that the patient is in a sleep state 1094. Although one external sensed physiologic parameter 250 and one internally sensed physiologic parameter are referenced, it is to be understood that two or more externally sensed physiologic parameters and/or two or more internally sensed physiologic parameters can be utilized in this example method. In some non-limiting examples, this mode is the “combination detection mode” discussed below with respect to FIGS. 20 and 22-23.

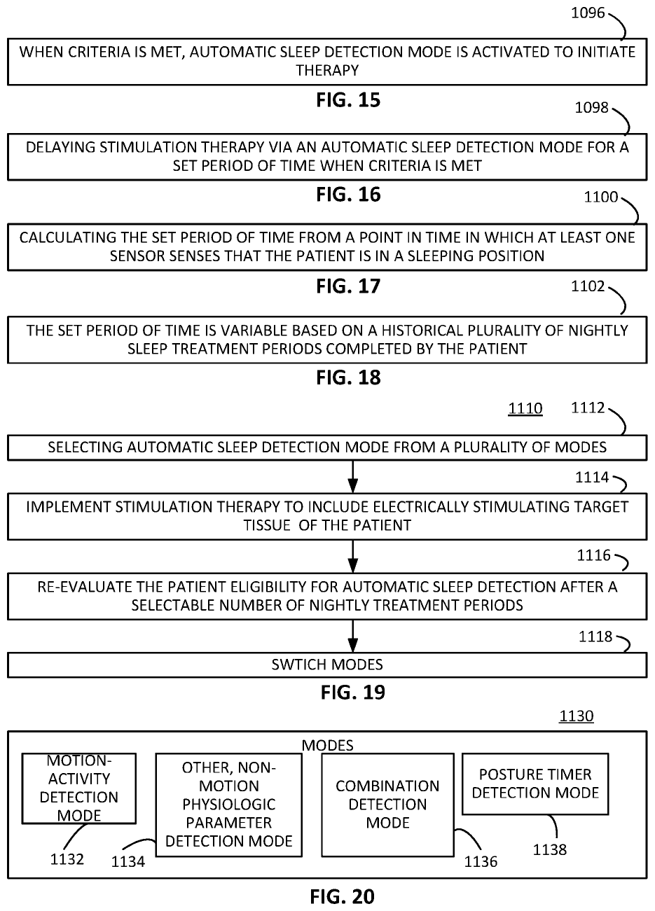

Referring in addition to FIG. 15, in view of the above, it can generally be described that automatic sleep detection mode can be activated to initiate therapy upon the occurrence of criteria being met, as shown at 1096. In one example, the criteria may relate to those indicating a designation for the patient as disclosed above. In some examples the criteria may include a plurality of metrics that need to be met in order for the criteria to be met. In yet another example, a single metric may be evaluated to determine if the criteria is met. Further, referring in addition to FIG. 16, it may be desirable to delay the start of stimulation therapy via any automatic sleep detection modes of the disclosure for a set period of time when criteria is met 1098. Referring in addition to FIG. 17, the set period of time is calculated from a point in time in which at least one sensor senses that the patient is in a sleeping position (any sleeping position disclosed herein) or the like 1100. Referring in addition to FIG. 18, in some examples, the set period of time may be calculated based on a historical plurality of nightly sleep treatment periods completed by the patient. 1102. The historical plurality of nightly sleep treatment periods can include a number of nightly sleep treatment periods of two or more, three or more, seven or more, fourteen or more, thirty or more, sixty or more, and ninety or more, for example. In one non-limiting example, the set period of time is between 1 and 20 minutes. In another example, the set period of time is between 1 and 30 minutes.

As suggested above, an automatic sleep detection mode can be one of a plurality of different modes, including different automatic sleep detection modes. Referring in addition to FIG. 19, methods 1110 of the disclosure can include selecting automatic sleep detection mode from a plurality of modes 1112, implementing therapy 1114 and further re-evaluating the patient eligibility for automatic sleep detection after a selectable number of nightly treatment periods 1116. In some examples, the therapy may comprise SDB therapy such as (but not limited to) electrically stimulating upper airway patency-related tissue of the patient.

This process may alternatively be called a calibration period or process. In some examples, re-evaluation will include repeating any of the aforementioned method steps. For example, the method steps shown described in association with at least FIGS. 2-4 may be repeated as part of the calibration process. If conducted, this calibration period can optionally serve one or more purposes; the first is to set weights and parameters for the patient based on measured/sensed signals and known sleep times, measured through patient logs, an external monitoring system, or the like. Another purpose is to re-evaluate the currently applied protocol/mode. After re-evaluation, it could potentially be evident that the patient would be better suited being regrouped and/or treated with an alternate automatic sleep detection mode or even initiating a manual automatic sleep detection mode (see also, FIG. 32 and related disclosure). In examples where automatic sleep detection mode is not imitated, such re-evaluation may result in automatic sleep detection mode being later initiated. In the aforementioned examples, the methods include initiating a first mode and switching to a second mode that differs from the first mode 1118. It is further envisioned that such re-evaluation steps may indicate that changes to the currently applied protocol are not recommended to change. In this case, the mode would not be switched or changed after re-evaluation.

Referring now in addition to FIG. 20, which illustrates various exemplary and non-limiting modes 1130 that can be applied as one of the plurality of automatic sleep detection modes of the disclosure. One mode is a motion-activity detection mode 1132. In an example of the motion-activity detection mode 1132, one or more sensors (e.g. 217, 250) are configured to look for a lack of movement or activity as an indication that the patient likely is in a sleep status. Referring in addition to FIG. 21, in such an example, automatic sleep detection mode 1132 is dependent on accelerometer derived information 1140 to identify patient movement. In various examples of motion-activity detection mode 1132, a motion-activity sensor (e.g. an accelerometer or gyroscope in some examples) may be exclusively or solely used in making a sleep determination. In some examples, when the motion/activity detected by the sensor is below a threshold such as, but not limited to, a lack of motion, the motion/activity detection mode 1132 will initiate SDB stimulation therapy.

Another automatic sleep detection mode is an other, non-motion-activity physiologic parameter detection mode 1134. In an example of the other physiologic parameter detection mode 1134, one or more sensors (e.g. 217, 250) can include a sensor configured to sense physical parameters or physiologic information (see also, FIG. 6 and related disclosure), which can be used to at least partially determine when a patient is asleep. For example, some sensed physiological information such as, but not limited to, cardiac signals/information (e.g. lowered heart rate), lowered temperature, respiratory signals/information (e.g. lowered respiration rate), etc. may be indicative of a sleep state. When a determination of a sleep state is made, physiologic parameter detection mode 1134 will initiate therapy (e.g. SDB therapy). In such an example, automatic sleep detection mode is dependent on physiologic parameter sensor derived information.

Yet another automatic sleep detection mode is a combination detection mode 1136. In one example of the combination detection mode 1136, at least two different sensed physiologic parameters (e.g. 217, 250, two of which can be internal and/or externally sensed) can include a motion-activity parameter and other (non-motion-activity) physiologic parameter, which together may permit at least partially determining a sleep state via a lack of movement (e.g. motion-activity) and other physiological indications (e.g. lowered heart rate, etc. as noted above) of a sleep state. When a sleep state is indicated by both of the motion-activity physiologic parameter and other physiologic parameter(s), combination detection mode 1136 may initiate therapy. Referring in addition to FIG. 22, in such an example, combination detection mode 1136 is dependent on motion-activity sensor-derived and other physiologic parameter sensor derived information 1142. In various examples, multiple sensor inputs are used for determining a patient's sleep-wake status. Optionally, sensor inputs can be weighted or otherwise prioritized when making a determination utilizing multiple sensor inputs. In another example, the combination detection mode 1136 can integrate a timer to delay therapy for a period of time after both sensed parameters indicate the patient is in a sleep state. In some examples, the period of time is variable/adjustable.

Yet another automatic sleep detection mode is a posture timer detection mode 1138. In an example of the posture timer detection mode 1138, one or more sensors (e.g. 217, 250) can include a posture sensor and a timer, which is configured to at least partially determine a sleep state of a patient via sensing the patient's posture for a given period of time. The one or more sensors may include an accelerometer in some examples. When a sleep state is determined based on posture sensed by the one or more sensors (e.g. a patient has been sensed as not changing posture for a period of time, such as 30 minutes, for example, or a patient has been sensed as laying down for a predetermined, set period of time), posture timer detection mode 1138 may initiate therapy. The set period of time can be variable and can optionally be dependent on the patient's sleep history (see also history 2542 of FIG. 61A). In some examples, the set period of time can be set by a clinician. In some examples, the set period of time could change (get shorter, for example) after each change in posture is detected. For example, if a patient lies down, a timer for a set period of time begins. If the patient moves to lay down on another side, the timer is extended 15 minutes. If the patient again moves to lay down on another side, the timer is again extended another 15 minutes. In another example, the timer is extended for shorter and shorter periods of time (upon each change in posture) until the timer runs down to zero and, at zero, sleep is detected.

Referring now also to FIG. 23, which indicates that methods of the disclosure can include determining the at least one variability metric based on a plurality of parameters. In some examples, each of the plurality of the parameters can be assigned a weight 1144. In some examples, the method of FIG. 23 is conducted during a re-evaluation process 1116 as additionally discussed above.

Referring now in addition to FIG. 24, which depicts example parameters or variability metrics 1150 that are non-limiting examples of parameters that can be utilized to determine one or more variability metrics of the disclosure. As shown, the one or more parameters can include, but are not limited to, sleep onset latency for one night 1152 (i.e. one nightly sleep treatment period), a mean sleep onset latency 1154, variability in sleep onset 1156, quantity of outliers 1158, and/or severity of outliers 1159, as previously described in in association with at least FIGS. 1-23. In addition, one parameter may include a quantity of pauses of the SDB stimulation therapy during one night 1160. In various examples, the stimulation element (e.g. 217) can be configured to be controlled such that the patient can pause stimulation therapy, via a remote, mobile device, sensed tap or the like, in the case that the patient wakes up briefly (see also, FIG. 64 and related disclosure). In other examples, sleep onset latency variability metrics may comprise one or more parameters, which can include sleep onset latency for a plurality of nightly sleep treatment periods and/or variability in sleep onset 1162. In another example, the sleep onset latency variability metric is a measure of time. In other words, the sleep onset latency variability metrics is a number of seconds or minutes (or other time scale) in which the patient takes to fall asleep.

Referring additionally to FIG. 25, when a sleep onset latency variability metric is obtained, some methods will include initiating automatic sleep detection mode when the sleep onset latency variability metric is below a threshold 1170. The threshold may be set at a number of standard deviations from the mean onset of sleep latency. In this instance, the sleep onset latency variability metric would indicate a relatively low variability in onset of sleep latency. In various examples, the threshold may be variable, dynamic depending on how well the systems of the disclosure are able to determine when a patient is in a sleep status and/or wake status.

Conversely, referring now in addition to FIG. 26, when the sleep onset latency variability metric is obtained, some example methods will include initiating a sleep detection mode when the sleep onset latency variability metric is above a threshold 1172. The threshold may be set at a number of standard deviations from the mean onset of sleep latency. In this instance, the sleep onset latency variability metric would indicate a relatively high variability in onset of sleep latency. Therefore, in some examples, when a sleep onset latency variability metric is above a threshold, a first mode may be enabled (e.g. standard initiation mode as described in FIG. 32) and when a sleep onset latency variability metric is below a threshold, a second mode may be enabled (e.g. an automatic sleep detection mode of the disclosure). Other modes may be designated for implementation above/below the threshold depending on the perceived effectiveness for a particular mode for a particular patent based on the sleep latency variability metric. It is further envisioned that any threshold or the criteria may be dynamic and change over time.

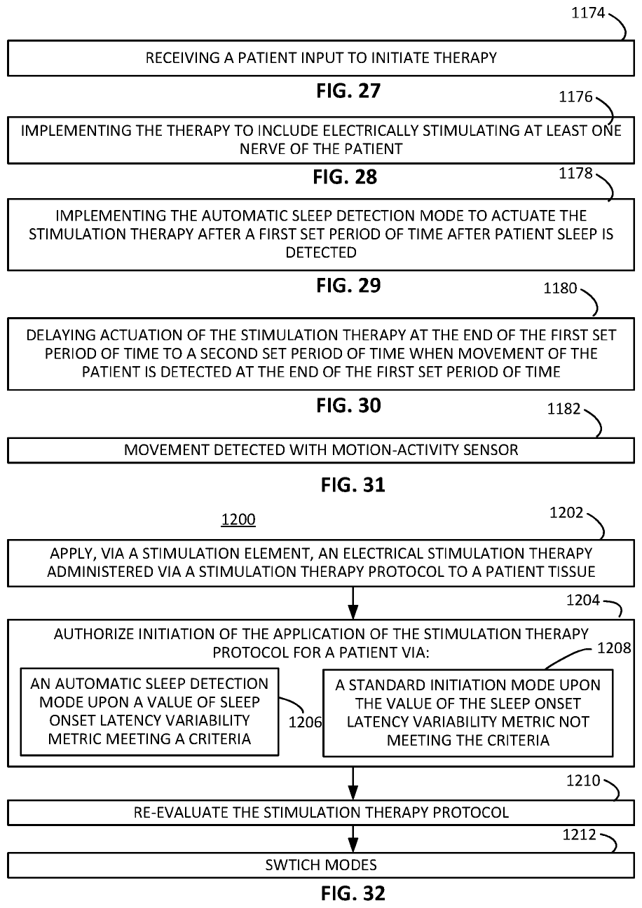

Referring in addition to FIG. 27, which at 1174 illustrates that in any of the methods disclosed herein, the methods may, for example, include a step of receiving a patient input, via remote, mobile device, sensed tap/motion or the like, to initiate therapy (e.g. SDB therapy). In one example, the patient input may specify that the patient is going to sleep. In some examples, the methods and systems of the disclosure may be configured to wait an amount of time, predetermined from historical data or otherwise, after the patient input is received to confirm the onset of sleep via one or more sensing modalities of the disclosure as the patient will not be in a sleep state at the moment the patient input is received.

Referring now in addition to FIG. 28, any of the methods of the disclosure can include implementing the therapy, such as sleep disordered breathing (SDB) stimulation therapy to include electrically stimulating upper airway patency-related tissue (e.g. at least one nerve and/or more muscle) of the patient 1176. Electrical stimulation can be of any of the type disclosed herein for any purpose disclosed herein and, further, the upper airway patency-related tissue can be any of those disclosed herein, for example.

Referring now also to FIG. 29, which illustrates that in some example methods, automatic sleep detection mode 1132 (FIG. 20) is implemented to actuate the therapy after a first set period of time after patient sleep is detected 1178. Optionally, the first set period of time can be measured with the delay timer of the disclosure or the like. Optionally, this first set period of time is clinician adjustable. Referring in addition to FIG. 30, in some examples, actuation of the therapy (e.g. SDB therapy) is delayed until the end of the first set period of time to a second set period of time when movement of the patient is detected at the end of the first set period of time 1180. In other words, the first set period of time can be extended in the instance where sensor(s) indicate that the patient is not yet asleep at the end of the first set period of time. As some therapies (e.g. SDB stimulation therapy) are often not perceived as comfortable by many patients while awake, it can be desirable to ensure that a patient is truly asleep or in a deep state of sleep before therapy (e.g. SDB stimulation therapy) is activated and applied to one or more nerves. Delaying therapy (e.g. SDB stimulation) for the first set period of time can accomplish this objective. In one non-limiting example, the first set period of time is between 1 and 120 minutes. In another non-limiting example, the first set period of time is between 1 and 30 minutes. Referring now in addition to FIG. 31, in one examples, sleep may be detected, for the purpose of starting the first set period of time, with one or more sensors, which can include a motion-activity sensor 1182, which may comprise an accelerometer or other motion-activity sensor in some examples. Optionally, the accelerometer or other motion-activity sensor can be utilized for other purposes relating to the therapy and/or selection/implementation of automatic sleep detection mode, which can be any mode of the disclosure (e.g. see FIG. 20 and related disclosure). For instance, the accelerometer may be used to sense heart rate, respiratory information (including respiratory rate), body position (e.g. posture), etc. as previously described herein.

Referring in addition to FIG. 32, which illustrates various methods 1200 of the disclosure. In various examples, methods 1200 include applying, via a stimulation element (e.g. stimulation element 217), an electrical stimulation therapy administered via a stimulation therapy protocol to patient tissue 1202. In some examples, the patient tissue is an upper airway patency-related tissue. Various methods can also include authorizing the initiation of the application of the stimulation therapy protocol for a patient 1204 via one or more of an automatic sleep detection mode 1206 of the disclosure upon a value of sleep onset latency variability metric meeting a criteria; or a standard initiation mode 1208 upon the value of the sleep onset latency variability metric not meeting the criteria. In one example, the standard initiation mode 1208 can be configured to initiate stimulation therapy with the physical manipulation of a button or switch (e.g. via a patient remote control) but it is further envisioned that standard initiation mode 1208 can be considered to include automatic initiation based on time of day (e.g. 10 p.m.) or via a delay timer. Methods can optionally further include re-evaluating the stimulation therapy protocol 1210 via any technique of the disclosure to determine if changes in the value of the sleep onset latency variability metric changes to meet the criteria. In the case where criteria is later met or no longer met, at 1212 in FIG. 32 methods can include switching modes from an automatic sleep detection mode 1206 or standard initiation mode 1208 or vice versa.

Referring now in addition to FIG. 33, as also discussed above with respect to FIG. 26, in some examples, the criteria 1220 is the value of sleep onset latency variability metric being below a threshold 1222. In other examples, the criteria is the value of sleep onset latency variability metric being above a threshold 1224.

The method 1200 of FIG. 32 can be integrated with any and all other aspects of the disclosure, including, but not limited to, FIG. 34 in which the method 1200 includes receiving recorded sleep onset latency information for a historical period of nightly sleep treatment periods for any time period disclosed herein 1230. As indicted in FIG. 35, the method 1200 of FIG. 32 can additionally include determining the sleep onset latency variability metric via sensing, via at least one sensor (e.g. sensor 217, 250) sleep-related physiologic information of any of the type of the disclosure for a plurality of nightly sleep treatment periods 1232. Referring in addition to FIG. 36, in some examples, the method 1200 of FIG. 32 includes actuating the stimulation therapy after a set period of time after a sleep status is detected via any means of the disclosure 1234. Additionally, in further reference to FIG. 37, in some examples, the patient provides an input to indicate a nightly treatment period is about to begin prior to the step of applying electrical stimulation therapy 1236.

It is to be understood that any thresholds or criteria of the disclosure can be variable, improved or modified throughout the method as performance of the protocols are evaluated and improved either through additional development or training/constructing a data model (e.g. artificial intelligence/machine learning) as discussed in additional detail below (see, e.g. data model 3230 below).

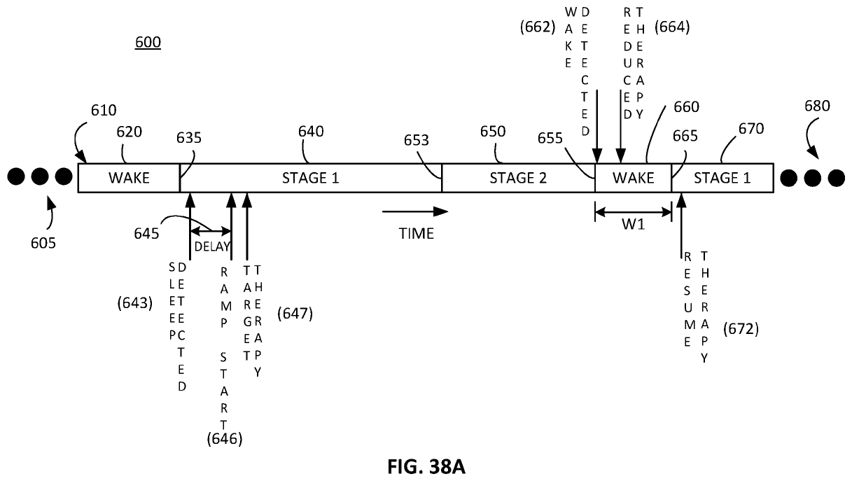

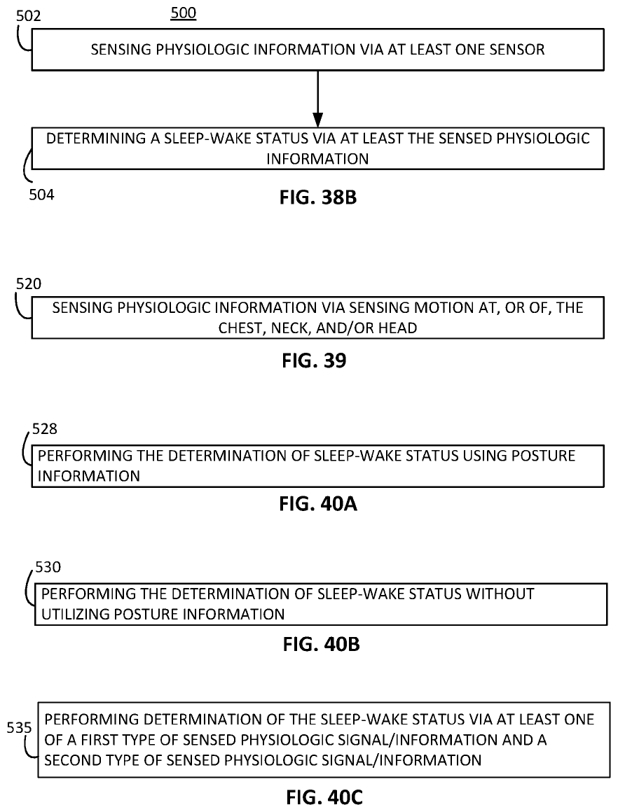

Referring in addition now to FIGS. 38A-68, which illustrate various optional devices and techniques for accomplishing any of the aforementioned functions, steps and/or processes. Among other things, as shown in FIG. 38B, the examples of FIGS. 38A-68 provide for example implementations of an example method 500 comprising, at 502, sensing physiologic information via at least one sensor and at 504, determining a sleep-wake status based on the sensed physiologic information.

In some examples, determining a sleep-wake status and/or determining sleep onset latency information (including onset latency variability) may be implemented without posture or body position information. Accordingly, in some examples, sleep in a particular posture or particular position is not used to determine sleep-wake status and/or to determine various aspects of sleep onset latency. For instance, even though a patient may be in positions other than a supine position or side-laying position, example methods and/or devices of. In the present disclosure may still determine a sleep-wake status, sleep onset latency, etc. Accordingly, example methods and/or devices may provide more robust and more accurate determination of sleep-wake status, sleep onset latency, etc., and therefore provide more useful automatic initiation and/or termination of treatment periods regardless of sleep posture. In some examples, the automatic initiation, automatic pause features, etc. (e.g. arising from automatically detecting a sleep-wake status) may be selectively activated or deactivated by a clinician or patient, such as via a clinician programmer or a patient remote control (e.g. see also FIG. 64 and related disclosure).

FIG. 38A is a diagram schematically representing a timeline 610 of sleep-wake-related events according to an example method 600 of sleep-wake determination, such as may occur during sleep disordered breathing (SDB) care (e.g. monitoring, diagnosis, treatment, etc.). In some examples, the example SDB care may comprise at least some of substantially the same features and attributes as the example SDB care methods and/or devices (including sleep-wake detection) as described in association with FIGS. 1-37 and 38B-68.

As shown in FIG. 38A, the timeline 610 includes a series of wake and sleep periods with wake period 620 occurring just before a first sleep stage period 640 (e.g. stage 1). The wake period 620 in FIG. 38A may represent an end portion of a wake period extending since the end of a prior night's sleep or may represent another wake period.

As further represented by indicator 635, a real physiologic transition occurs between the wake period 620 and the first sleep stage 640 and indicator 643 represents a detection of sleep according to examples of the present disclosure. As shown in FIG. 38A, the detection of sleep 643 may occur just after the physiologic transition 635.

In some examples, the detection of sleep 643 may trigger a delay period 645 prior to a start of therapy (e.g. electrical stimulation). In some such examples, the duration of the delay generally corresponds to an amount of time sufficient for a patient to experience sufficiently sound sleep such that the patient will not be awakened by the onset of stimulation. Moreover, in some examples, once stimulation begins it may be implemented in a ramped manner 646 with an initial lower stimulation intensity which is gradually increased until a target stimulation intensity (647) is achieved to therapeutically provide electrical stimulation to an upper airway patency related tissue.

As previously noted in the present disclosure, at least some example implementations of method 600 in FIG. 38A may comprise identifying, maintaining, and/or optimizing a target stimulation intensity (e.g. therapy level) without intentionally identifying a stimulation discomfort threshold at the time of implantation or at a later point in time after implantation.

As further shown in FIG. 38A, once the target stimulation intensity is achieved, it may be maintained throughout the treatment period.

In some examples, the target stimulation intensity may be automatically adjusted (e.g. auto-titrated) during the treatment period. In some such examples, the automatic adjustment of the target stimulation intensity may be implemented according to at least some of substantially the same features and/or attributes as described in association with at least auto-titration parameter 2920 in FIG. 61A.

As further shown in FIG. 38A, after some period of time (which may vary from night to night) the patient may sometimes experience a wake period 660 during a treatment period, which interrupts a sleep stage (e.g. a second sleep stage (S2) 6050 in this example). The example method 600 detects wakefulness (662), which may extend for a period of time (W1), before the patient goes back to sleep, such as represented by sleep stage 670 and transition 665 between the respective wake period 6060 and sleep stage 670.

In some examples, method 600 may completely pause stimulation during the wakeful period 660 or instead in some examples, method 600 may implement a reduced therapy 664 during the wakeful period 660 because of the expectation of the patient going back to sleep and a full stimulation therapy being resumed. In some such examples, the reduced therapy at 664 may comprise providing stimulation at a functional threshold (FT), which corresponds to a minimum amplitude at which the stimulation will cause the tongue to protrude at least part way past the lower teeth and at which a therapeutic outcome (e.g. reduction in apneas) may be achieved. However, in some such examples, the reduced therapy at 664 may comprise providing stimulation at a sensation threshold (ST), which involves a stimulation intensity less than the stimulation intensity to reach the functional threshold (FT). The sensation threshold (ST) may correspond to a minimum amplitude at which the patient can sense stimulation.

As indicated at 672 in FIG. 38A, therapy may be resumed automatically. It will be understood that, in at least some examples, the resumption of therapy 672 may comprise substantially the same features and attributes as the initiation of therapy as previously described in relation to indicators 643, 646, 647 in FIG. 38A, including detection of sleep 643 according to at least some of the examples of the present disclosure to determine a sleep-wake status.

In some examples, in general terms, the beginning of the first sleep stage 640 generally corresponds to a beginning of a treatment period during which a patient may be treated for sleep disordered breathing and/or the method (and/or device) may monitor for or diagnose sleep disordered breathing.

More specific example methods, devices, and/or arrangements of determining a sleep-wake status (including sleep onset latency information and related information) are described and illustrated in association with least FIGS. 39-68.

As schematically represented at 520 in FIG. 39, in some examples sensing the physiologic information may comprise sensing motion at, or of, the chest, neck, and/or head, which in turn may be used to determine the sleep-wake status. At least some aspects of such determination are further described in association with FIGS. 58-61A. For instance, sensing portion 2000 in FIG. 58A and/or care engine 2500 (including but not limited to sensing portion 2510) in FIG. 61A comprises multiple sensor types, modalities, etc., at least some of which may be used to sense motion at, or of, the chest, neck, and/or head, and to utilize such sensed motion to determine a sleep-wake status (e.g. detecting sleep). One such example modality may comprise employing an accelerometer to sense motion at the chest, neck, and/or head, as further described later. In some examples, the accelerometer may be implanted at the chest, neck, and/or head, while in some examples, the accelerometer may be secured externally on the patient's body at such locations.

The sensed motion at the chest, neck, and/or head may comprise motion of the chest, neck, and/or head or may comprise motion phenomenon at those respective locations without necessarily involving gross motion of the chest, neck, and/or head, as further described later in association with at least FIGS. 60A-60G. In one non-limiting example, sensing the motion phenomenon at the neck or other location may comprise sensing circulation of blood within a blood vessel/vasculature (e.g. arterial motion within a vessel). In some such examples, the sensing element (e.g. accelerometer, impedance, other) may be at least partially incorporated in a microstimulator (or other implantable pulse generator) sized and shaped to be implantable within a blood vessel. The example method may comprise sensing ballistic motion of the blood vessel caused by the heartbeat of the patient. In some examples, the blood vessel may comprise an external jugular vein and hence the sensing of motion may occur at the neck in some examples without necessarily being motion of the neck (e.g. bending, titling, twisting, etc.).

As schematically represented at 528 in FIG. 40A, in some examples, a method of determining a sleep-wake status may be performed using sensed posture information and/or body position information. The sensed posture information may comprise a static posture or may comprise a change in posture, which may be considered a form of gross body motion mentioned above. As noted elsewhere, the sensed posture may be used to help confirm whether the patient is likely sleeping (e.g. lying down) or awake (e.g. sitting up) which may be in combination with other sensed information (e.g. heart rate, respiratory rate, etc.).

As schematically represented at 530 in FIG. 40B, in some examples, a method of determining a sleep-wake status may be performed without utilizing posture information and/or body position information.

For instance, a patient may sometimes intentionally (or unintentionally) sleep when sitting in a chair or an airline seat, and would benefit from SDB care (e.g. neurostimulation therapy). In such instances, determining sleep-wake status without using posture information may enhance quicker or more accurate detection of sleep for the patient sleeping in a sitting position because the example method may avoid a false negative indication (by a posture-based determination) that the patient is awake.

Conversely, a patient may sometimes intentionally be awake when lying horizontally, and accordingly, does not wish to receive SDB care. In such instances, determining sleep-wake status without using posture information may enhance quicker or more accurate detection of sleep for the patient who is awake in a lying-down position because the example method avoids a false positive indication (by a posture-based determination) that the patient is asleep because they are laying in the horizontal position typically associated with sleep.

As schematically represented at 535 in FIG. 40C, in some examples, a method of determining a sleep-wake status comprises sensing at least one of a first type of physiologic signal/information (e.g. respiratory signal, from which respiratory rate and/or other information may be derived and/or a cardiac signal, from which heart rate and/or other information may be derived) and a second type of physiologic signal/information (e.g. body movement), and performing determination of the sleep-wake status at least via at least one of the respective first type of sensed physiologic signal/information and the second type of sensed physiologic signal/information. In some examples, the sensed body movement may correspond to the sensed motion in FIG. 39. Various aspects of determination the sleep-wake status based on such sensed physiologic information is further described in association with at least FIGS. 58-61A and elsewhere throughout the various examples of the present disclosure.

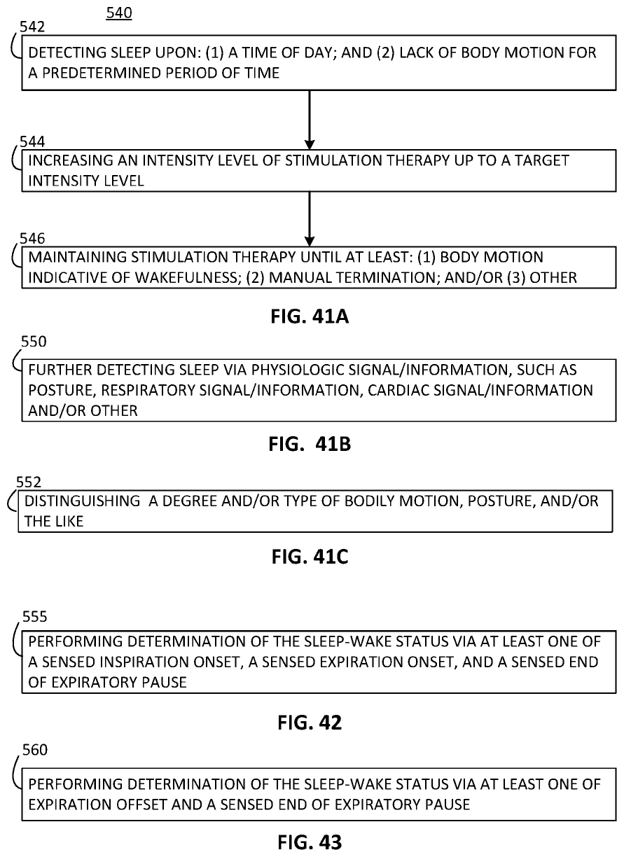

In some examples detecting sleep (and/or wakefulness) in association with delivering a stimulation therapy may comprise the method shown at 540 in FIG. 41A. As shown at 542 in FIG. 41A, the method 540 may comprise detecting sleep upon: (1) a time of day; and (2) detection of a lack of bodily motion indicative of sleep over a selectable, predetermined period of time. The time-of-day may be selectable and/or based on patient data. Once at least these two criteria are met, then as shown at 544 in FIG. 41A, the method comprises increasing the intensity of the stimulation therapy from a lower initial intensity level to a target intensity level, such as in a ramped manner. As long as sensed physiologic information indicates that sleep is continuing, then stimulation at the target intensity level continues. However, upon the detection of body motion by the patient (which is indicative of wakefulness) or upon detection of the patient mechanically indicating wakefulness (e.g. physically tapping on chest near IPG), then the method may terminate any stimulation therapy and may remain in a no-stimulation mode for a selectable predetermined of time (e.g. 15 minutes). In other words, after the interruption, the method may delay therapy onset for set period of time (e.g. 15 minutes). The length of the delay period is programmable.

As shown 550 in FIG. 41B, in some examples the method 540 may further comprise sensing onset of sleep via additional physiologic signals/information, such as sensing posture, respiratory signals/information (e.g. stability regarding period, depth, etc.), cardiac signals/information (e.g. stability per R-R interval, HR, etc.), and/or other information. For instance, in one non-limiting example, portion 542 of method 540 may comprise detecting posture (550) and comprise detection of sleep for some particular postures (but not others) and/or for some particular changes in posture (but not others). In some examples, the particular postures and/or particular changes in posture may be selectable by a patient and/or clinician. For example, the specified posture for which sleep is detectable may comprise a lying down posture (e.g. supine, left side, right side) but the method not permitting auto-detection of sleep when a patient is sitting up.

In some instances, the example methods may detect (e.g. recognize) REM sleep and thereby avoid a false positive detection of wakefulness. In particular, while respiration during REM sleep does not exhibit the same stability as in non-REM sleep, such sensed less-stable respiration may be confirmed as occurring during REM sleep (and not wakefulness) based upon the patient having been asleep for some extended period of time (e.g. passage through multiple sleep stages, S1-S4) and upon the patient exhibiting a lack of bodily motion (e.g. of the type of bodily motion one would observe in wakefulness).

Implementation of method 540 also may comprise enhancing sensitivity to and/or specificity regarding the physiologic phenomenon being sensed.

In some examples, the detection of sleep (e.g. at 542) in method 540 in FIG. 41A also may comprise distinguishing a degree and/or type of bodily motion, posture, and the like as shown at 552 in FIG. 41C. This distinguishing may be performed in association with ramping up stimulation (e.g. at 544), ramping down stimulation, terminating stimulation (e.g. 546), etc. For instance, via aspect 552 of method 540, the method may distinguish voluntary bodily motion as opposed to the jostling of the patient caused by vehicle motion (e.g. airplane, car, etc.) or by a bed partner. In some such examples, upon detecting such jostling, the method 540 may comprise temporarily decreasing stimulation therapy or pausing therapy, and then resuming the method at 544 to cause a quick return to target (e.g. therapeutic) intensity stimulation levels. In contrast, via aspect 552, the method 540 may identify physical tapping of the chest (near the IPG) as a voluntary bodily motion/cause or may identify a significant change to posture (e.g. change from lying down to sitting up) as being voluntary (e.g. not inadvertent) and then terminating therapy (or causing a longer pause) as at 546 in FIG. 41A because such detected behavior is indicative of wakefulness, whether temporary or longer term.

At least FIGS. 42-45 provide at least some example methods by which the determination of sleep-wake status may be made according to respiratory morphologic features. Moreover, at least some aspects of such sensing and related determination (of the sleep-wake status) relating to respiratory morphology features are further described in association with at least FIGS. 58 and 61A.

In one aspect, the various features of respiration morphologies addressed below in FIGS. 42-45 (e.g. inspiration onset, inspiration offset, magnitude, etc.) may enhance determining the sleep-wake status (e.g. at least sleep detection). In one aspect, these features of the respiratory morphology are readily identifiable and therefore beneficial to use in tracking a respiratory rate, which may be indicative of sleep (vs. wakefulness) according to the value of the respiratory rate, trend, and/or variability of the respiratory rate. In some examples, at least some of these features of respiration morphology may exhibit stability, which may be characteristic of sleep (vs. wakefulness). Some examples of such stability, which may be used to detect sleep/wake transitions, may include a stable respiratory rate, stability in an amplitude of the respiratory signal, stability of the percentage of the respiratory period corresponding to inspiration, and/or stability of the percentage of the respiratory period corresponding to expiration.

As schematically represented at 555 in FIG. 42, in some example methods, determining a sleep-wake status, such as via tracking at least some of the above-identified respiratory rate information, may comprise sensing at least one of an inspiration onset(s), an expiration onset(s), and end of expiratory pause, and performing determination of the sleep-wake status at least via at least one of the sensed inspiration onset(s), sensed expiration onset(s), and sensed end of expiratory pause.

As schematically represented at 560 in FIG. 43, in some example methods, determining a sleep-wake status, such as via tracking at least some of the above-identified respiratory rate information, may comprise sensing at least one of an expiration offset(s) and an end of expiratory pause(s) and performing determination of the sleep-wake status via at least one of the sensed expiration offset(s) and end of expiratory pause(s).

It will be understood that other combinations may be employed such as combining different combinations of fiducials (e.g. inspiration onset, end of expiratory pause, etc.) from FIGS. 42-45 or using just one of these fiducials from FIGS. 5-8 in determining a sleep wake status.

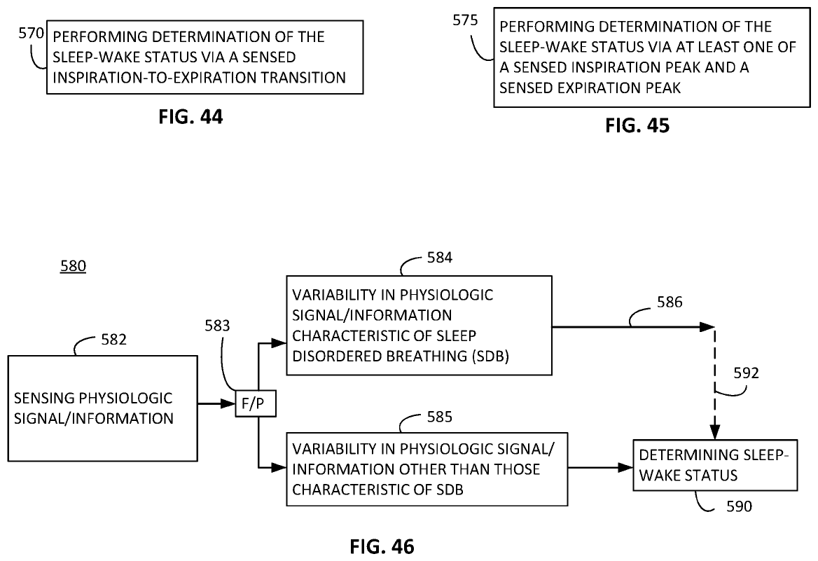

As schematically represented at 570 in FIG. 44, in some example methods, determining a sleep-wake status (such as via tracking at least some of the above-identified respiratory rate information) may comprise sensing an inspiration-to-expiration transition(s), and performing determination of the sleep-wake status at least via the sensed inspiration-to-expiration transition(s). Conversely, in some examples, sensing the physiologic information comprises sensing an expiration-to-inspiration transition(s), and determination of the sleep-wake status is performed via the sensed expiration-to-inspiration transition(s).

As schematically represented at 580 in FIG. 45, in some example methods, determining a sleep-wake status (such as via tracking at least some of the above-identified respiratory rate information) may comprise sensing at least one of an inspiration peak(s) and an expiration peak(s), and performing determination of the sleep-wake status via at least one of the sensed inspiration peak(s) and sensed expiration peak(s).

In some examples, at least some of the sensing of respiratory features, morphologies, etc. may be detected via sensing bioimpedance, as further described later in association with at least impedance parameter 2536 in FIG. 61A. Of course, as noted elsewhere, sensing of such respiratory features, etc. may be implemented via sensing modalities other than, or in addition to, sensing bioimpedance. For instance, in some examples, at least some of the sensing of respiratory features, morphologies, etc. may be detected via sensing an electrocardiogra information, as further described later in association with at least ECG parameter 2520 in FIG. 61A and/or 2020 in FIG. 58.

In some such examples associated with at least FIGS. 42-45 and/or at least FIGS. 46-48B, a method and/or device for determination of sleep-wake status via sensing variability in respiratory behavior, cardiac behavior, and/or other physiologic information may comprise identifying some features of such variability which are indicative of sleep disordered breathing (SDB) and differentiating the identified SDB-indicative features from other features of respiratory behavior, cardiac behavior, and/or other physiologic information such as those which are indicative of a sleep or wakefulness.

For instance, as schematically represented in the block diagram of FIG. 46, in some examples a method 580 (or device for) determining a sleep-wake status may comprise sensing physiologic signals/information (e.g. respiratory features and/or cardiac features) as shown at 582. At 583, method 580 may comprise applying filtering and processing (F/P) of the sensed physiologic signals/information to produce: (1) filtered/processed signal information at 584 comprising variability in physiologic signals/information (e.g. respiratory features and/or cardiac features) which are characteristic of sleep disordered breathing (SDB); and (2) filtered/processed signal information at 585 comprising variability in physiologic signals/information (e.g. respiratory features and/or cardiac features) other than those characteristic of sleep disordered breathing (SDB). Via the signal information at 585, at 590 the method may comprise determining sleep-wake status. In some such examples, the sleep-wake status determination may comprise at least some of substantially the same features as described in association with at least FIGS. 42-45 or other examples described throughout the present disclosure.

With further reference to FIG. 46, in some examples, the output 586 of the information 584 may be used in monitoring, diagnosing, treating, etc. sleep disordered breathing (SDB). However, in some examples, via path 592 this sensed physiologic signals/information (e.g. respiratory and/or cardiac information) characteristic of sleep disordered breathing (SDB) may be used to confirm determination of a sleep-wake status, such as confirming that the patient is in a sleep state by confirming the occurrence of sleep disordered breathing. In making this confirmation, the method may identify characteristics of sleep disordered breathing including (but not limited to) at least some of the periodic nature of SDB, such as the reoccurring sequence of a flow limitation, an apnea (or hypopnea), and recovery. This identification also may comprise identifying similar periodic changes in heart rate occurring without detecting any gross changes in posture.

Alternatively, the method may comprise at least partially confirming that the patient is in a wake state (which is primarily determined by other information) via confirming the absence of sleep disordered breathing, such as due to the periodic nature of changes to respiratory patterns and heart rate without gross posture changes.

In some examples, determination of a sleep-wake status may be performed via sensed cardiac morphological features. At least some aspects of such sensing and related determination (of the sleep-wake status) relating to cardiac morphology features are further described in association with at least FIGS. 58 and 61A.

Various features of cardiac morphologies may enhance determining the sleep-wake status (e.g. at least sleep detection) at least because these features of the cardiac morphology are readily identifiable and therefore beneficial to use in tracking a heart rate, which may be indicative of sleep (vs. wakefulness) according to value of, trend of, and/or the variability of the heart rate (HRV). In some examples, at least some of these features of cardiac morphology may exhibit increasing stability, which may be characteristic of sleep (vs. wakefulness). In some examples, at least some sleep stages may exhibit more or less variability in heart rate variability (HRV) and/or more or less variability in respiratory features, as noted above. For instance, more variability in cardiac features (e.g. heart rate, etc.) and respiratory features (e.g. respiratory rate, etc.) can be expected in REM sleep. At least some examples of determining a sleep-wake status may identify such variability in cardiac and respiratory signals characteristic of a REM sleep stage in a manner which can be distinguished from variability (or lack thereof in some instances) of cardiac and respiratory signals characteristic of wakefulness. For instance, when the sensing of a moderate increase in variability of respiratory and/or cardiac features follows other sleep stages (e.g. S3, S4) coupled with sensing a lack of body motion, then the example methods may identify that the patient in in REM sleep.

In some examples, at least some of the sensing of cardiac features, morphologies, etc. may be detected via sensing bioimpedance, as further described later in association with at least impedance parameter 2036 in FIG. 58 2536 in FIG. 61A. In some examples, at least some of the sensing of cardiac features, morphologies, etc. may be detected via sensing an electrocardiogra information, as further described later in association with at least ECG parameter 2020, 2520 in FIGS. 58 and 61A, respectively. The bioimpedance and/or ECG which is used to sense cardiac features, morphologies, etc., also may be used to sense respiratory features, morphologies, etc. (as previously noted), or may be used to sense both cardiac and respiratory features, morphologies, etc. It will be further understood that FIGS. 58, 61A provide additional example sensing types, modalities, etc. by which cardiac information (including but not limited to heart rate and/or heart rate variability) may be sensed, and which then may be used in determining sleep-wake status.

At least some of these relationships in cardiac morphology are further described in association with cardiac portion 2600 of care engine 2500 in FIG. 61A.

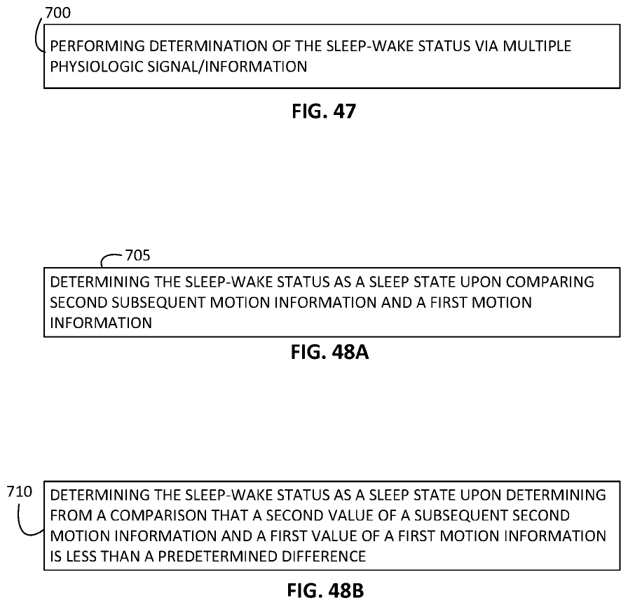

As schematically represented at 700 in FIG. 47, in some example methods, determining a sleep-wake status may comprise multiple physiologic signals/information (e.g. respiration information, cardiac information (e.g. heart motion)), and performing determination of the sleep-wake status via multiple physiologic signals/information (e.g. respiration information, cardiac information).

As schematically represented at 705 in FIG. 48A, in some example methods, determining a sleep-wake status (e.g. onset of sleep, etc.) may comprise comparing subsequent second motion information to first motion information. As further shown at 710 in FIG. 48B, in some examples, method 705 may comprise determining the sleep-wake status (e.g. onset of sleep, etc.) upon determining from the comparison that a second value of the subsequent second motion information and a first value of the first motion information is less than a predetermined difference. The value of the predetermined difference may be selectable.

In some examples of methods 705, 710, each of the respective first and second motion information comprises at least one of sensed respiratory information, sensed cardiac information, and sensed body motion.

In some examples of methods 705, 710, the subsequent second information comprises information obtained in the most recent sensed respiratory cycle and the first information comprises information obtained in a prior respiratory cycle. In some examples, the subsequent second information comprises information for respiratory activity in at least the last 30 seconds. In some examples, this information may relate to respiratory activity in at least the last 60 seconds. In some examples, this information may relate to respiratory activity in at least the last 7 breaths.

In some examples, the prior respiratory cycle comprises a respiratory cycle immediately preceding the most recent sensed respiratory cycle. In some examples, the prior respiratory cycle(s) comprise respiratory activity in the 30 seconds (or 60 seconds, or 7 breaths) preceding the most recent sensed respiratory cycle. In some examples, the first information comprises respiratory information over at least one respiratory cycle or at least 30 seconds or at least 60 seconds.

In some examples of methods 705, 710, recent motion information is compared to objective values indicative of sleep. In some examples, lower and/or more stable respiration rates and heart rates are more likely to be associated with sleep. As previously noted in association with at least FIG. 46, determining a sleep-wake status may comprise separating out (e.g. filtering, rejection) of respiratory features characteristic of sleep disordered breathing (SDB) and/or of respiratory features characteristic of particular sleep stages which do not necessarily contribute to general sleep detection (e.g. detecting onset of sleep).

However, as previously noted with respect to at least aspects 584, 592 in the method of FIG. 46, the detection of sleep disordered breathing (SDB) also may be used to sense or confirm the presence of sleep, or may be used to sense or confirm the onset of sleep in some instances.

In some examples, the method (at 705, 710 in FIGS. 48A-48B) may comprise determining the subsequent second motion information from a second average value of motion information in the respiratory cycles of the sensed second respiratory period and determining the first motion information from a first average value of motion information in the respiratory cycles of the first respiratory period. In some such examples, the second average value of motion information corresponds to an average of a parameter, such as but not limited to: an average amplitude of the sensed second respiratory period; an average respiratory rate of the sensed second respiratory period; and/or an average ratio of an inspiratory period relative to an expiratory period for the sensed second respiratory period.

It will be further understood that in some examples, the example implementations associated with FIGS. 48A-48B may be used for any biologic signal of interest which may contribute to determining sleep-wake status throughout the various examples of the present disclosure.

In some examples, at least some of the aspects described above with respect to FIGS. 48A-48B may be implemented via a history parameter 2542 and/or comparison parameter in sensing portion 2510 of care engine 2500, as later described in association with at least FIG. 61A.

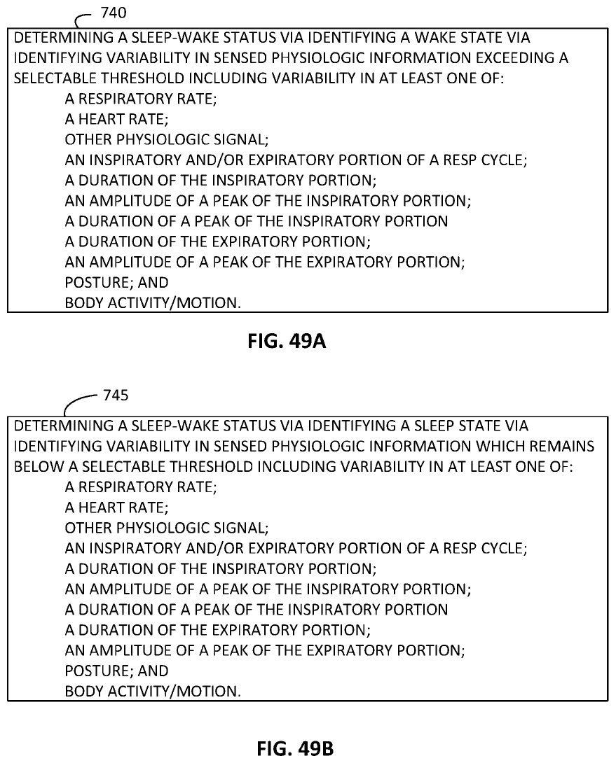

As schematically represented at 740 in FIG. 49A, in some example methods, determining a sleep-wake status may comprise identifying a wakefulness state (or lack thereof) via identifying variability in sensed physiologic information including variability in at least one of: a respiratory signal and/or information derived therefrom (e.g. a respiratory rate); a cardiac signal and/or information derived therefrom (e.g. a heart rate); other physiologic signal (e.g. EEG, ECG, EMG, EOG, etc.); an inspiratory and/or expiratory portion of a respiratory cycle; a duration of the inspiratory portion; an amplitude of a peak of the inspiratory portion; a duration of a peak of the inspiratory portion; a duration of the expiratory portion; body activity; and an amplitude of a peak of the expiratory portion. In some examples, for at least some parameters, the variability in sensed physiologic signals/information may be evaluated relative to a threshold, which may be fixed in some examples.

As schematically represented at 745 in FIG. 49B, in some example methods, determining a sleep-wake status may comprise identifying a sleep state (or lack thereof) via identifying variability in sensed physiologic information including variability in at least one of: a respiratory signal and/or information derived therefrom (e.g. a respiratory rate); a cardiac signal and/or information derived therefrom (e.g. a heart rate); other physiologic signal (e.g. EEG, ECG, EMG, EOG, etc.); an inspiratory and/or expiratory portion of a respiratory cycle; a duration of the inspiratory portion; an amplitude of a peak of the inspiratory portion; a duration of a peak of the inspiratory portion; a duration of the expiratory portion; body activity; and an amplitude of a peak of the expiratory portion. In some examples, for at least some parameters, the variability in sensed physiologic signals/information may be evaluated relative to a threshold, which may be fixed in some examples.

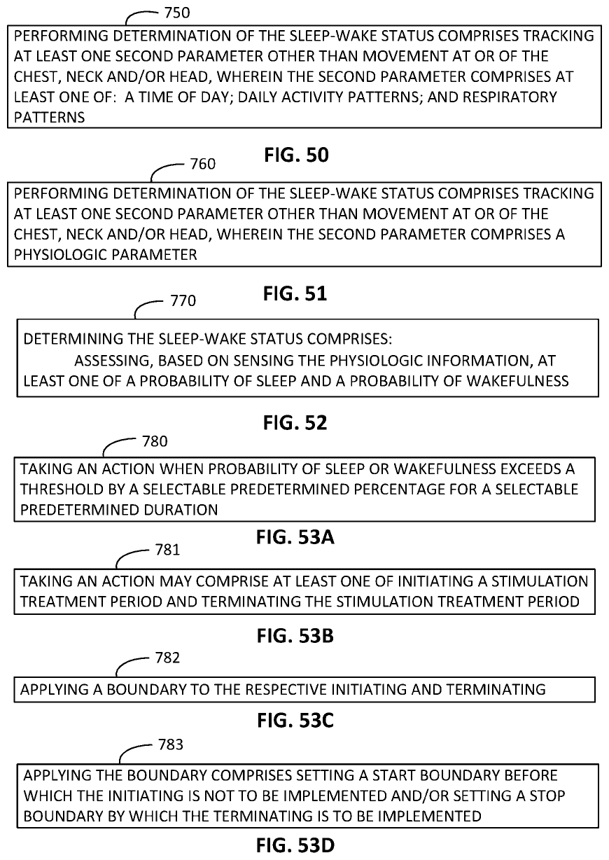

As schematically represented at 750 in FIG. 50, in some examples, performing determination of the sleep-wake status comprises tracking at least one second parameter other than movement at (or of) the chest, neck, and/or head, wherein the second parameter comprises at least one of: a time of day; daily activity patterns; and (typical) respiratory patterns.

As schematically represented at 760 in FIG. 51, in some examples, performing determination of the sleep-wake status comprises tracking at least one second parameter other than movement at (or of) the chest, neck, and/or head, wherein the second parameter comprises a physiologic parameter. For examples, one such physiological parameter may comprise temperature (e.g. 2038 in FIG. 58, 2538 in FIG. 61A).

As schematically represented at 770 in FIG. 52, in some examples, determining the sleep-wake status comprises assessing, based on sensing the physiologic information, at least one of a probability of sleep and a probability of wakefulness.

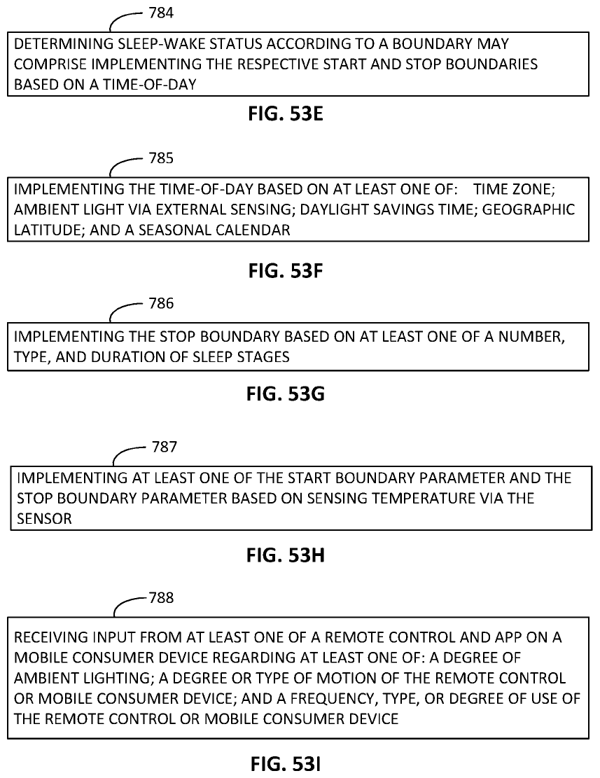

As schematically represented at 780 in FIG. 53A, some example methods (and/or devices) comprise taking an action when a probability of sleep or a probability of wakefulness exceeds a threshold. In some such examples, some example methods (and/or devices) comprise taking an action when a probability of sleep or a probability of wakefulness exceeds a threshold by a selectable predetermined percentage for a selectable predetermined duration.

In some examples of method 780 (FIG. 53A), taking an action may comprise at least one of initiating a stimulation treatment period and terminating the stimulation treatment period as shown at 781 in FIG. 53B. In some such examples, the taking an action (when a probability of sleep exceeds the threshold as in 780 in FIG. 53A) may comprise initiating a therapy treatment period (e.g. applying stimulation), resuming stimulation within a treatment period after a pause or suspension of stimulation, and/or other actions. In some examples, taking an action (when a probability of wakefulness exceeds the threshold) may comprise terminating a therapy treatment period, suspending stimulation within a treatment period, and/or other actions.

In some such examples, the initiating and/or resuming stimulation therapy may comprise employing a stimulation ramp in which an initial stimulation intensity is lower and then increased to a target intensity level. In some examples, terminating therapy may comprise employing a stimulation ramp in which a stimulation intensity is decreased gradually from a target therapy intensity level until stimulation is no longer applied (i.e. stimulation intensity equals zero).

With further reference to at least FIG. 53A, in some examples taking an action in method 780 may comprise use of an observer for an additional period of time to ensure the patient is asleep and/or using a start timer to initiate counting a selectable, predetermined period of time (e.g. delay) until stimulation is initiated as part of a treatment period.

In some such examples as method 780 (FIG. 53A), the method further comprises applying a boundary to the respective initiating and terminating as shown at 782 in FIG. 53C. At least some aspects of such a boundary are further described in association with boundary parameter 3016 of activation portion 3000 in FIG. 61A.

In some example methods associated with method 780, applying the boundary comprises setting a start boundary before which the initiating is not be implemented and/or setting a stop boundary by which the terminating is to be implemented, as shown at 783 in FIG. 53D.

In some examples, the method (e.g. 782, 783) of determining sleep-wake status according to a boundary may comprise implementing the respective start and stop boundaries based on a time-of-day, as shown at 784 in FIG. 53E. In some examples, as shown at 785 in FIG. 53F, the method may comprise implementing the time-of-day based on at least one of: time zone; ambient light via external sensing; daylight savings time; geographic latitude; and a seasonal calendar.

In some examples, as shown at 786 in FIG. 53G, the method (e.g. 783) may comprise implementing the stop boundary based on at least one of a number, type, and duration of sleep stages.

In some examples, as shown at 787 in FIG. 53H, the method (e.g. 783) may comprise implementing at least one of the start boundary parameter and the stop boundary parameter based on sensing temperature via the implantable sensor. In some examples, the method may comprise implementing, at least one of the initiating of the stimulation treatment period and the terminating of the stimulation treatment period, based on sensing body temperature via the implantable sensor. In some examples, the method may comprise arranging the implantable sensor within an implantable pulse generator and the implantable sensor comprises a temperature sensor. In some such examples, method 787 may be implemented via, and/or is further described later in association with, at least temperature sensor 2038 in FIG. 58, temperature parameter 2538 in FIG. 61A, and/or at least boundary parameter 3016 in FIG. 61A.

In some examples, as shown at 788 in FIG. 53I, the method (including determining the sleep-wake status such as at 770 in FIG. 52 and/or in 780 at FIG. 53A) may comprise receiving input from at least one of a remote control and app on a mobile consumer device regarding at least one of: a degree of ambient lighting; a degree or type of motion of the remote control or mobile consumer device; and a frequency, type, or degree of use of the remote control or mobile consumer device.

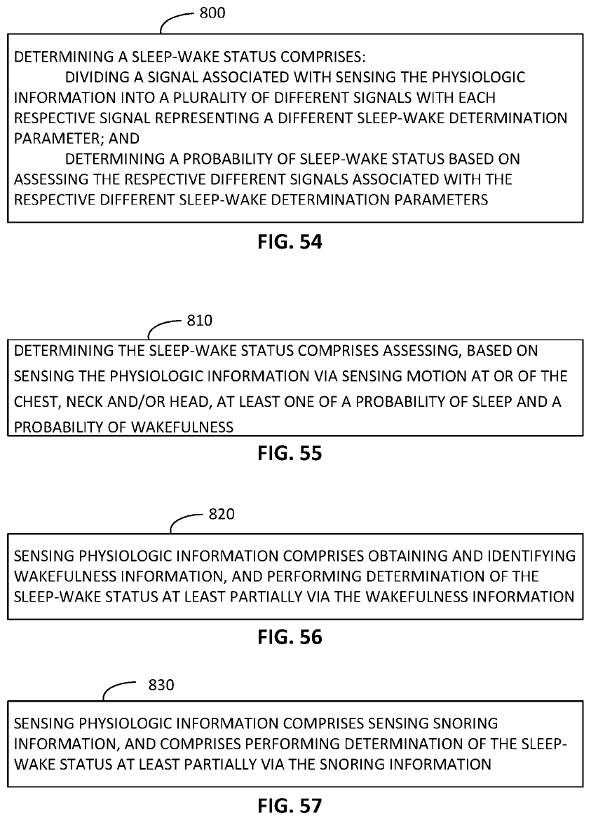

As schematically represented at 800 in FIG. 54, some examples of determining a sleep-wake status may comprise: dividing a signal associated with sensing the physiologic information into a plurality of different signals with each respective signal representing a different sleep-wake determination parameter; and determining a probability of sleep-wake status based on assessing the respective different signals associated with the respective different sleep-wake determination parameters. In some examples, this example method may comprise voting, by which each signal provides input to the overall probability of sleep. In some such examples, the various separate signals may be weighted differently so as to apply each respective sleep-wake determination parameter relatively more or relatively less in comparison to the other respective sleep-wake determination parameters.

In some examples, at least some aspects of method 800 (FIG. 31) may be implemented via at least some of the features and attributes of the arrangement described in association with at least FIGS. 58-61A.

As schematically represented at 810 in FIG. 55, in some examples determining the sleep-wake status comprises at least one of: assessing, based on sensing the physiologic information via sensing motion at (or of) the chest, neck, and/or head, at least one of a probability of sleep and a probability of wakefulness.

As schematically represented at 820 in FIG. 56, in some examples, sensing physiologic information comprises obtaining and identifying wakefulness information (e.g. during normal wake periods), and comprising performing determination of the sleep-wake status at least partially via the wakefulness information. In some such examples, the wakefulness information is used to better characterize sleep and therefore more readily determine a sleep-wake status (e.g. such as detecting sleep or lack thereof). However, in this context, the identified wakefulness information is not used to adjust therapy (e.g. stimulation parameters, etc.) and/or not used to characterize a respiratory disorder. In some examples, the identification of wakefulness may be performed via sensing at least one of gross body motion and movement. In some examples, sensing physiologic information comprises obtaining sleep information, and comprising performing determination of the sleep-wake status via the sleep information.

As schematically represented at 830 in FIG. 57, in some examples, a method comprises sensing snoring and using the snoring information as part of determining sleep-wake status. In some such examples, the method(s) may comprise quantifying the sensed snoring, and reporting snoring information to at least one of a patient, physician, or caregiver. In some examples, the snoring may be differentiated from normal speech. In some examples, snoring may be sensed, tracked, etc. in association with acoustic sensor 2039 (FIG. 58) and/or acoustic parameter 2539 (FIG. 61A).

In some examples, various features and attributes of the example methods (and/or care devices) described in association with at least FIGS. 1A-1D, 38-57 for determining sleep-wake status may be combined and implemented in a complementary or additive manner.

These, and additional features and attributes associated with FIGS. 1A-57 will be further described in association with at least FIGS. 58-61A. Moreover, at least some of the examples described in association with FIGS. 58-68 may comprise example implementations of the examples described in association with FIGS. 1A-57.

FIG. 58 is a block diagram schematically representing an example sensing portion. In some examples, an example method may employ and/or an example SDB care device may comprise the sensing portion 2000 to sense physiologic information and/or other information, with such sensed information relating to sleep-awake detection, among other uses. The sensed information may be used to implement at least some of the example methods and/or examples devices described in association with at least FIGS. 1A-57 and/or FIGS. 59-68.

It will be understood that the sensing portion 2000 may be implemented as single sensor or multiple sensors, and may comprise a single type of sensor or multiple types of sensing. In addition, it will be further understood that the various types of sensing schematically represented in FIG. 58 may correspond to a sensor and/or a sensing modality.

In some examples, the sensed information may refer to physiologic signals (e.g. biosignals) and/or information (e.g. metrics) which may be derived from such physiologic signals. For instance, among other sensed physiologic information, one example of physiologic information may comprise respiration (2005) obtained from a respiratory signal and from which various information may be derived such as, but not limited to, respiratory rate, respiratory rate variability, respiratory phase, rate times volume, waveform morphology, and more. The respiration information and/or signal may be sensed via one or more sensing modalities described below (and/or other sensing modalities) such as, but not limited to, accelerometer 2026, ECG 2020, EMG 2022, ballistocardiogram 2023A, seismocardiogram 2023B, accelerocardiogram 2023C, impedance 2036, pressure 2037, temperature 2038, acoustic 2039, and/or other sensing modalities, at least some of which are further described below. In some examples, the sensed physiologic information may comprise cardiac information (2006) obtained from a cardiac signal and from which various information (e.g. metrics) may be derived such as, but not limited to, heart rate (HR), heart rate variability (HRV), P-R intervals, waveform morphology, and more. One example of a cardiac signal may comprise an ECG signal, as represented at 2020 in FIG. 30A. Accordingly, the cardiac information and/or signal may be sensed via one or more sensing modalities described below (and/or other sensing modalities) such as, but not limited to, accelerometer 2026, ECG 2020, EMG 2022, impedance 2036, pressure 2037, temperature 2038, acoustic 2039, and/or other sensing modalities, at least some of which are further described below. In some examples, the sensed physiologic information (e.g. via sensing portion 2000) may comprise a wide variety of physiologic information other (2007) than respiration and/or cardiac information, with at least some examples further described below in association with FIG. 30A, 32, and other examples throughout the present disclosure.

The sensed physiologic signals and/or information (e.g. respiration 2005, cardiac 2006, and/or other information 2007) may be used for a wide variety of purposes such as, but not limited to, sleep-wake status (e.g. various sleep onset determinations), timing stimulation relative to respiration, disease burden, arousals, etc. In some such examples, the detection of disease burden may comprise detection of sleep disordered breathing events, which may be used in determining, assessing, etc. therapy outcomes such as, but not limited to, AHI, as well as titrating stimulation parameters, adjusting sensitivity of sensing the physiologic information, etc.

For instance, in one non-limiting example, an electrocardiogram (ECG) sensor 2020 in FIG. 58 may comprise a sensing element (e.g. electrode) or multiple sensing elements arranged relative to a patient's body (e.g. implanted in the transthoracic region) to obtain ECG information. In some examples, the ECG information may comprise one example implementation to obtain cardiac information, including but not limited to, heart rate and/or heart rate variability (HRV), which may be used (with or without other information) in determining sleep-wake status as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g. outer housing) of an implantable pulse generator (IPG), such as further described later in association with at least FIG. 60A. In such instances, other ECG electrodes are spaced apart from the ECG electrode associated with the IPG. In some examples, such as further described in association with FIG. 60A, at least some ECG sensing electrodes also may be employed to deliver stimulation to a nerve or muscle, such as but not limited to, an upper airway patency-related nerve (e.g. hypoglossal nerve) or other nerves or muscles.

In some examples, multiple ECG sensing electrodes may be mounted on or form different portions of a case of an IPG, such as later described in association with at least FIGS. 60C, 60D, 60E. In such examples, the respective ECG electrodes are arranged on the case of the IPG to be electrically independent of each other so that a suitable ECG signal may be obtained.

In some examples, an ECG sensing electrode may be used solely for sensing (e.g. single purpose) but is located along a lead body of a stimulation lead, as further described later in association with FIG. 60A. It will be understood that such dedicated ECG sensing electrode is positioned along the stimulation lead in a manner to avoid contact with a case of the IPG, particularly in examples in which an exposed electrically conductive portion of the case of the IPG may act as an electrode and by which a sensing vector may be obtained via a combination of the sensor electrode along the lead and the conductive portion of the IPG. Similarly, the same/similar electrode arrangement may be used to sense bioimpedance, as also described more fully later in association with FIGS. 60A-60G, 61A.

In some examples, other types of sensing may be employed to obtain cardiac information (including but not limited to heart rate and/or heart rate variability), such as via ballistocardiogram sensor(s) 2023A, seismocardiogram sensor(s) 2023B, and/or accelerocardiogram sensor(s) 2023C as shown in FIG. 58. In some examples, such sensing is based on and/or implemented via accelerometer-based sensing such as further described below in association with accelerometer 2026.

In one aspect, in some examples the ballistocardiogram sensor 2023A senses cardiac information caused by cardiac output, such as the forceful ejection of blood from the heart into the great arteries that occurs with each heartbeat. The sensed ballistocardiogram information may comprise heart rate (HR), heart rate variability (HRV), and/or additional cardiac morphology. In some examples such ballistocardiogram-type information may be sensed from within a blood vessel in which the sensor (e.g. accelerometer) senses the movement of the vessel wall caused by pulsations of blood moving through the vessel with each heartbeat. This phenomenon may sometimes be referred to as arterial motion.

In one aspect, the seismocardiogram sensor 2023B may provide cardiac information which is similar to that described for ballistocardiogram sensor 2023A, except for being obtained via sensing vibrations, per an accelerometer (e.g. single or multi-axis), in or along the chest wall caused by cardiac output. In particular, the seismocardiogram measures the compression waves generated by the heart (e.g. per heart wall motion and/or blood flow) during its movement and transmitted to the chest wall. Accordingly, the sensor 2023B may be placed in the chest wall.

In some such examples of sensing per sensors 2023A, 2023B, such methods and/or devices also may comprise sensing a respiratory rate and/or other respiratory information.

As further shown in FIG. 58, in some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-neck region otherwise suitable to sense EEG information. Accordingly, the EEG sensor(s) 210 are located near the brain and may detect frequencies associated with electrical brain activity.

In some examples, a sensing element used to sense EEG information is chronically implantable, such as in a subdermal location (e.g. subcutaneous location external to the cranium skull), rather than an intracranial position (e.g. interior to the cranium skull). In some examples, the EEG sensing element is placed and/or designed to sense EEG information without stimulating a vagus nerve at least because stimulating the vagal nerve may exacerbate sleep apnea, particularly with regard to obstructive sleep apnea. Similarly, the EEG sensing element may be used in a device in which a stimulation element delivers stimulation to a hypoglossal nerve or other upper airway patency nerve without stimulating the vagus nerve in order to avoid exacerbating the obstructive sleep apnea.

In some examples the sensing portion 2000 may comprise an electromyogram (EMG) sensor 2022 to obtain and track EMG information. In some such examples, the EMG sensor may comprise an electrode positioned near the tongue to detect signals indicative of voluntary control of the tongue, which in turn may be indicative of wakefulness. In some examples, the sensed EMG signals may be used to identify sleep and/or obstructive events. At least some additional aspects regarding EMG sensing is described in association with at least FIG. 60A.

In some examples, as shown in FIG. 58, the sensing portion 2000 may comprise an EOG sensor 2024 to obtain and track EOG information, which may be used to a determine sleep-wake status and/or different sleep stages. In some instances, such sensed EOG information may be used to distinguish REM sleep from non-REM sleep or from wakefulness. In some examples, a sensing element for obtaining EOG information may be implanted in the head-and-neck portion, such as adjacent the eyes, eye muscles, and/or eye nerves, etc. In some examples, the sensing element may communicate the EOG information wirelessly, or via an implanted lead, to a control element (e.g. monitor, pulse generator, and the like) implanted within the head-and-neck region. In some such examples, the sensing element may comprise an electrode implanted near one or both eyes of the patient.

However, in some examples, the EOG information may be obtained via external sensing elements which are worn on the head or which may observe the eye movement, position, etc. such as via a mobile phone, monitoring station within proximity to the patient, and the like. Such externally-obtained EOG information may be communicated wirelessly to an implanted monitor, pulse generator and the like which controls sensing elements and/or stimulation elements implanted within the patient's body. Some aspects of sensing via EOG sensor are further described later in association with at least FIG. 61A.

In some examples, any one or a combination of the various sensing modalities (e.g. EEG, EMG, etc.) described in association with FIG. 34A may be implemented via a single sensing element 2014.

In some examples, the sensing portion 2000 may comprise an accelerometer 2026. In some examples, the accelerometer 2026 and associated sensing (e.g. motion at (or of) the chest, neck, and/or head, respiratory, cardiac, posture, etc.) may be implemented according to at least some of substantially the same features and attributes as described in Dieken et al., ACCELEROMETER-BASED SENSING FOR SLEEP DISORDERED BREATHING (SDB) CARE, published as U.S. 2019-0160282 on May 30, 2019, and which is incorporated by reference herein in its entirety. In some examples, the accelerometer may comprise a single axis accelerometer while in some examples, the accelerometer may comprise a multiple axis accelerometer.

Among other types and/or ways of sensing information, the accelerometer sensor(s) 2026 may be employed to sense or obtain a ballistocardiogram (2023A), a seismocardiogram (2023B), and/or an accelerocardiogram (2023C), which may be used to sense (at least) heart rate and/or heart rate variability (among other information such as respiratory rate in in some instances), which may in turn may be used as part of determining sleep-wake status as described throughout the examples of the present disclosure.

In some examples, the accelerometer 2026 may be used to sense activity, posture, and/or body position as part of determining a sleep-wake status, the sensed activity, posture, and/or body position may sometimes be at least partially indicative of a sleep-wake status.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g. electrodes) spaced apart from each other across a portion of the patient's body, such as electrodes 2120, 2135, 2130 in FIG. 60A, and/or example electrodes (e.g. 2310, 2402, 2404) in FIGS. 60C-60G. In some such examples, one of the sensing elements (e.g. electrode 2135 in FIG. 60A) may be mounted on or form part of an outer surface (e.g. case) of an implantable pulse generator (IPG) or other implantable sensing monitor, while other sensing elements (e.g. electrodes 2120, 2130 in FIG. 60A) may be located at a spaced distance from the sensing element of the IPG or sensing monitor. In at least some such examples, the impedance sensing arrangement integrates all the motion/change of the body (e.g. such as respiratory effort, cardiac motion, etc.) between the sense electrodes (including the case of the IPG when present). Some examples implementations of the impedance measurement circuit will include separate drive and measure electrodes to control for electrode to tissue access impedance at the driving nodes.

In some examples, the sensing portion 2000 may comprise a pressure sensor 2037, which senses respiratory information, such as but not limited to respiratory cyclical information. In some such examples, the respiratory pressure sensor may comprise at least some of substantially the same features and attributes as described in Ni et al., US Patent Publication US2011/0152706, METHOD AND APPARATUS FOR SENSING RESPIRATORY PRESSURE IN AN IMPLANTABLE STIMULATION SYSTEM, published on Jun. 23, 2011, and which is incorporated herein by reference in its entirety. In some examples, the pressure sensor 2037 may be located in direct or indirect continuity with respiratory organs or airway or tissues supporting the respiratory organs or airway in order to sense respiratory information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiratory information, snoring, etc.

In some examples, sensing portion 2000 may comprise body motion parameter 2026 by which patient body motion may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities, as described throughout various examples of the present disclosure.

In some examples, the sensing portion 2000 in FIG. 58 may comprise a posture parameter 2040 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine sleep-wake status. As described elsewhere herein, in some examples posture may be considered as one of several parameters when determining a probability of sleep (or awake).

For instance, sensing an upright posture typically is associated with a wakeful state, such as standing or walking. However, as noted elsewhere, a person could be in an upright sitting position and still be in a sleep state (e.g. sleeping a chair). Accordingly, posture may be just one parameter used in determining a sleep-wake state, along with at least some other parameters described in association with sensing portion 2000 of FIG. 58 and/or care engine 2500 in FIG. 61A. Conversely, sensing a supine or lateral decubitis (i.e. laying on a side) posture typically is associated with a sleep state. However, a patient might be in such a position without being asleep, such that other parameters (e.g. FIGS. 58, 61A) in addition to, or instead of, posture may significantly enhance determination of sleep-wake status.

Moreover, sensing posture may not be limited to sensing a static posture but extend to sensing simple changes in posture (or body position), which may be indicative of a sleep-wake state at least because certain changes in posture (e.g. from supine to upright) are mostly likely indicative of a wake state. Similarly, more complex or frequent changes in posture and/or body position may be further indicative of a wake state, whereas maintaining a single stable posture for an extended period time may be indicative of a sleep state.

In some examples, the sensing portion 2000 of care engine 2500 (FIG. 61A) comprises other parameter 2041 to direct sensing of, and/or receive, track, evaluate, etc. sensed information other than the previously described information sensed via the sensing portion 2510.

In some example methods and/or devices, via sensing portion 2000, a sleep-wake status may be determined without using posture information or body position information. In some such examples, a determination of a sleep-wake status without regard to posture information (or body position) may permit the device to provide efficacious sleep disordered breathing (SDB) care even when the patient may be sleeping in a vertical position, such as sitting in a chair, in a zero-gravity environment, etc. in contrast to a conventional assumption of sleep occurring in a horizontal body position. Such implementations may permit SDB care when a patient is sleeping during travel, such as sitting in an airplane seat, automobile seat, train seat, etc. In some such examples, a SDB care method and/or SDB care device may sometimes be referred to as being posture-insensitive.

As further shown in FIG. 58, in some examples the sensing portion 2000 may comprise a temperature sensor 2038. In some examples, such sensed temperature may be tracked, evaluated, etc. in association with temperature parameter 2538 in sensing portion 2510 of care engine 2500 in FIG. 61A.

In some examples, the sensed temperature may be used as one factor in making a sleep-wake status determination according to the examples of the present disclosure. In one aspect, the temperature sensor 2038 may sense and track a patient's normal fluctuation (e.g. temperature profile) in body temperature within a 24 hour daily period, which may exhibit on the order of a 2 degree F. change. For most patients, their body temperature may reach and remain at the high end (e.g. 99.5 F) of its range during the middle of the day and evening (e.g. 7 pm) before falling throughout late evening and overnight to the low end (e.g. 97.5 F) of its range by early morning (e.g. 5 or 6 am). In some examples, the temperature sensor 2038 may sense a change in the sensed temperature which occurs within a selectable time window of a 24 hour daily period and which exceeds a selectable threshold. In some examples, the selectable time window may comprise one hour, two hours, or other time periods. In some such examples, one method comprises selecting that a change of a predetermined number of degrees within the selectable time window will correspond to either a wake-to-sleep state transition or a sleep-to-wake state transition.

In some example methods, sensing a change in temperature (such as via sensor 2038) during a treatment period may be used to identify sleep disordered breathing behavior. In some such examples, additional sensed information (as described in examples of the present disclosure) may be used in addition to sensed temperature to identify sleep disordered breathing (SDB) behavior.

In some examples, this temperature fluctuation information se sensed via temperature sensor 2038 may be used in association with boundary parameter 3016 to automatically implement a boundary or limit on the beginning and end of the treatment period, such that the lowest sensed body temperature may be used to at least partially mark a boundary of an end of a treatment period for a typical patient which sleeps at night. Similarly, the highest sensed body temperature (e.g. held for an extended period) may be used to at least partially implement a boundary at a beginning of a treatment period. In some such examples, these features may be used to implement method 787 in FIG. 20H.

In some examples, these same temperature-based boundaries may be used as one factor (among other factors) to determine a sleep-wake status. At least some other factors, which may be used with this sensed temperature fluctuation information to determine a sleep-wake status, may comprise a time of day parameter, accelerometer information, cardiac information, respiratory information, etc.

In some examples, smaller yet detectable temperature changes within a treatment period may be used to at least partially determine a sleep-wake status. For instance, a detectable temperature change may be sensed as a result of patient exertion to breathe in response to an apnea event, given the greater muscular effort in attempting to breathe.

Moreover, in some examples, such sensed temperature fluctuation information may provide a more distinctive or characteristic indication of a sleep or wake period when compared with heart rate or body position, which may exhibit more changes, some of which are not necessarily indicative of a sleep period or wake period, at least in some instances.

In some examples, at least some of the sensors and/or sensor modalities described in association with FIG. 58 (and/or FIG. 61A) may be incorporated within or on a pulse generator (IPG 2133 in FIG. 60A), or within or on a microstimulator (e.g. FIGS. 60A-60F).

FIGS. 60A-64 include diagrams schematically representing several example implementations of sensing elements, stimulation devices, related components, care engines, etc. for treating a patient, which may be employed in, may comprise an example implementation of, and/or may comprise at least some of substantially the same features and attributes as, of at least some of the example methods and/or example devices described throughout the present disclosure. In particular, in some examples, at least some aspects of the examples associated with FIGS. 60A-64 may comprise an example implementation of, may comprise at least some of substantially the same features and attributes as, and/or may include (or be exchanged with) additional/other elements from the various components, functions, relationships of at least the examples in association with at least FIGS. 1A-1C.

FIG. 60A is a diagram schematically representing several example implementations of sensing elements and a neurostimulation device 2113 implanted with a patient. As shown in FIG. 60A, the neurostimulation device 2113 may comprise an implantable pulse generator (IPG) 2133 and stimulation lead 2117, which comprises a lead body 2118 and a stimulation electrode 2112. The stimulation electrode 2112 is subcutaneously implanted and engaged relative to an upper airway patency-related nerve 2105, such as the hypoglossal nerve. In some examples, the IPG 2133 is implanted in the pectoral region 2101 with stimulation lead 2117 extending upward into the head-and-neck region 2103. In some examples, the stimulation electrode 2112 is chronically implantable, and may comprise a cylindrical arrangement to be at least partially wrapped about a nerve, may comprise a paddle-style electrode, may comprise a non-cuff configuration, or other configuration by which electrode may be chronically implanted in nerve-stimulating relation to the nerve.

In some examples, the stimulation electrode 2112 may comprise at least some of substantially the same features and attributes as described in Bonde et al. U.S. Pat. No. 8,340,785, SELF EXPANDING ELECTRODE CUFF, issued on Dec. 25, 2102 and Bonde et al. U.S. Pat. No. 9,227,053, SELF EXPANDING ELECTRODE CUFF, issued on Jan. 5, 2016, both which are hereby incorporated by reference in their entirety. In some examples, the stimulation electrode 2112 may comprise at least some of substantially the same features and attributes as described in Johnson, U.S. Pat. No. 8,934,992 NERVE CUFF, issued on Jan. 13, 2015, and/or in Rondoni, CUFF ELECTRODE, published as WO 2019/032890 on Feb. 14, 2019 (and filed as U.S. application Ser. No. 16/485,954 on Aug. 14, 2019), both which are hereby incorporated by reference in their entirety. Moreover, in some examples the stimulation lead 2117 may comprise at least some of substantially the same features and attributes as the stimulation lead described in U.S. Pat. No. 6,572,543 to Christopherson et al., and which is incorporated herein by reference.

However, it will be understood that in some examples the IPG 2133 also may take the form of a microstimulator, which is sized and placed, in the head-and-neck region 2103 in close proximity to the upper airway patency-related nerve 2105 to be stimulated. In some such examples, the microstimulator 2133 also may incorporate and/or include the stimulation electrode 2112 and/or sensing electrodes. In some example implementations in which the IPG 2133 may comprise a microstimulator, then placement of the microstimulator in the head-and-neck region 2103, such as in close proximity to the upper airway patency-related nerve would also place any exposed electrodes (e.g. 2135) on microstimulator in closer proximity to the nerve 2105, as well as in closer proximity to the head portion 2105 from which EEG information (including sleep information) may be determined via such electrode 2135. In some examples, such example microstimulators may comprise at least some of substantially the same features and attributes as described in association with at least MICROSTIMULATION SLEEP DISORDERED BREATHING (SDB) THERAPY DEVICE, published on May 26, 2017 as PCT Publication WO 2017/087681 from application PCT/US2016/062546 filed on Nov. 17, 2016, and filed as U.S. application Ser. No. 15/774,471 on May 8, 2018, both of which are which is incorporated herein by reference. In such example arrangements including a microstimulator as the IPG 2133, the stimulation lead 2117 may be omitted (while still retaining stimulation electrode 2112) or the stimulation lead 2117 may be significantly shortened.

Via such neurostimulation device(s) (2133, 2112), delivery of a stimulation signal to the upper airway patency-related nerve 2105 may cause contraction of at least some upper airway patency-related muscles (e.g. the genioglossus muscle, infrahyoid strap muscles, etc.) to cause at least protrusion of the tongue (and/or other muscular actions) to maintain, increase, or restore upper airway patency, and thereby provide therapeutic treatment of obstructive sleep apnea. At least some further example implementations regarding such stimulation are described in association with at least FIGS. 61A-68.

In some examples, the example microstimulator may be implanted in the head-and-neck region (e.g. 2103) of a patient to sense at least some of the desired sleep-wake-related information, which may be used to perform sleep-wake determination. In some examples, sleep-wake determination may be used to implement, control, adjust, etc. therapy of sleep disordered breathing per neurostimulation of upper-airway-patency related nerves, muscles, tissues, etc. At least some example implementations of a head-and-neck implanted microstimulator may take the form described later in association with at least FIGS. 60C-60G.

In some examples, such as described later in FIGS. 60C-60G, the device implanted within the head-and-neck region may comprise a sensing element forming a part of and/or associated with the microstimulator. In some such examples, the sensing element(s) may be used to determine sleep-wake via detection of cardiac signals such as heart rate based on ECG or arterial motion. In some examples, the sensing element(s) may be used to determine sleep-wake via detection of respiratory signals such as respiratory motion or the subset of such motions that could be considered sounds including, but not limited to, snoring. In some such examples, the sensing element(s) may detect both cardiac signals and respiratory signals.

In some examples, whether involving microstimulation or involving other implantable pulse generators (IPG 2133 in FIG. 60A), changes to sensed signals after and/or during stimulation may be used to quantify therapy effectiveness and/or may be used to implement auto-titration of the stimulation, as further described later in association with at least FIG. 61A.

In some examples, the stimulation electrode 2112 also may serve as a sensing element to sense physiologic information. In some such examples, the electrode 2112 may act as the sole sensing element to sense the physiologic information, such as a single channel EEG electrode or a single channel ECG electrode or other sensing modalities per sensing portion 2000 (FIG. 58A) or sensing portion 2500 (FIG. 61A). As noted below, in some examples the stimulation electrode 2112 may be used for sensing in combination with other sensing elements and/or sensing modalities.

In some examples, the stimulation lead body 2118 may comprise a sensing element (e.g. electrode) 2120, which may act as the sole sensing element to sense the physiologic information, such as cardiac information, EEG information, EMG information, movement information, etc. in accordance with sensing portion 2000 (FIG. 58), 2500 (FIG. 61A). Accordingly, in some examples, the sensing element 2120 may comprise an accelerometer. However, in some examples a sensing element (e.g. electrode) 2120 may be considered the sole sensing element when used in association an electrically conductive exterior portion (e.g. at least part of a case/housing) of an implantable stimulator (e.g. IPG or microstimulator).

In some examples, a single/sole sensor may comprise a pressure sensor (e.g. 2037 in FIG. 58), and in some examples, pressure sensed via sensor 2037 may being tracked, evaluated, etc. via pressure parameter 2537 in sensing portion 2510 of care engine 2500 in FIG. 61A.

In some examples, the EMG information sensed via one of the electrodes (e.g. 2120, 2112, etc.) may comprise detecting upper airway patency to assess obstruction (e.g. degree, location, etc.) and/or assess stimulation effectiveness, as well as detecting (and/or assessing) inhalation/exhalation during respiration. In some examples, the sensed EMG information may comprise sensing intercostal muscle activity in order to identify respiratory cyclical information (e.g. inspiration, expiration, expiratory pause) and/or identify or differentiate between central sleep apnea and obstructive sleep apnea.

However, in some examples, one or both of electrode 2112 and electrode 2120 may be used in association with other sensing elements (e.g. electrodes) to sense physiologic information.

In some examples, IPG 2133 comprises a sensing element(s) 2135. In some such examples, the sensing element 2135 is located on a surface of (or forms) a case of IPG 2133, and one of both of electrodes 2112 and 2120 may be used in association with electrode 2135 to measure bio-impedance (2036 in FIG. 58; 2536 in FIG. 61A), to obtain an ECG signal, an EMG signal, etc. sense cardiac information (including cardiac morphology), respiratory information (including respiratory morphology), motion/movement of the chest, neck, and/or head, etc.

In some such examples, the sensing element 2135 of the IPG 2133 may comprise an accelerometer, which may comprise a single axis or multiple-axis accelerometer. The accelerometer may be located internally within the IPG 2133, may be located externally on the IPG 2135, or may extend a short distance from the IPG 2133 via a small lead body.

As discussed in association with at least parameters 2026, 2526 in FIGS. 58 and 61A, respectively, the accelerometer may be employed to sense motion at (or of) the chest, neck, and/or head, cardiac information, respiratory information, etc. In some examples, the accelerometer may be used to sense body activity/movement/motion, such as gross body motion (e.g. walking, talking), which may be indicative of activity associated with wakefulness. Alternatively, sensing a lack of activity via an accelerometer may be indicative of a sleep state, in some examples. In some such examples, the accelerometer may be used to sense physiologic information for use in at least some of the example methods of determining sleep-wake status without being used to sense posture or body position, as previously described herein. However, in some examples, the accelerometer may be used to sense such posture or body position.

With further reference to FIG. 60A, in some examples of determining a sleep-wake status, an electrode 2110 may be implanted subdermally in a head portion 2105 of a head-and-neck region 2103 (e.g. above the shoulder) to sense electrical brain activity and obtain EEG information and/or other central nervous system (CNS) information, with such sensed information being used to determine the sleep-wake status. Among other aspects, sleep onset, sleep termination, and/or various sleep stages may be determined via the sensed EEG information. In some examples, multiple electrodes 2110 may be placed subdermally about the head portion 2015 to sense such EEG information. In some examples, a single electrode 2110 may be used in combination with another electrode, such as the stimulation electrode 2112, to sense such EEG information. In some examples, the electrode 2110 may comprise the sole sensing element used to determine sleep-wake status.

In some example methods and/or devices of determining a sleep-wake status, an electrode 2114 may be implanted in or in close proximity to a tongue 2115 to sense electromyography (EMG) information. This sensed EMG information may include signals which are indicative of voluntary control of the tongue (e.g. talking, eating, etc.), which in turn may be indicative of wakefulness. In addition, this sensed EMG information may include signals which are indicative of sleep and/or sleep disordered breathing (e.g. obstructive events) such as when the tongue may relax into a position obstructing the upper airway.

It will be understood that just some of the various electrodes shown in FIG. 60A may be implanted or present in a particular example implementation. Moreover, while some of the electrodes (if present) may be used in combination with each other, some of the electrodes may be used to implement a particular sensing modality periodically or selectively rather than all of the time. For instance, there may be periods of time in which some electrodes are used to sense one modality (e.g. cardiac information, such as an ECG or other), while some electrodes are used to sense another modality (e.g. impedance) during some periods of time, with such periods of time being overlapping, coincidental, or independent of each other.

With this in mind, in some examples, one or multiple sensing modalities for determining wake-sleep status may be implemented in some instances while another, a different sensing modality (or a different combination of sensing modalities) may be implemented in other instances. For example, certain sensing modalities may be employed solely or less significantly during a portion of the daily period (e.g. normal wake period, such as 6 a.m. to 10 p.m.) and then not employed at all or less significantly during another portion of the daily period (e.g. normal sleep period), or vice versa.

In some example methods and/or devices, the normal wake period may be identified via at least one of clinician input, patient input, machine learning, and other observational criteria. In the example of clinician input or patient input, a user may directly specify the start time and/or end time of the normal wake period (and conversely the normal sleep period). In some examples, the normal wake period (or conversely the normal sleep period) may be at least partially determined via historical data for a particular patient and/or historical data regarding multiple patients or the general population. In some such examples, machine learning (e.g. machine-learning parameter 3230 in FIG. 61A) may be applied to the historical data to make the determination. In some such examples, the machine learning may be on-going on a daily basis using at least the most recent historical data (e.g. last 30 days).

In some examples, as described later, a probability of sleep (or the sleep-wake status) may be determined from among a plurality of sleep-wake status parameters in which different sleep-wake status parameters may be weighted differently. Such different weighting for a given sleep-wake status parameter may depend on a time-of-day, clinician/patient input, etc.

As schematically represented in FIG. 60B, in some examples the IPG 2133 of FIG. 60A may comprise a plurality of sensing elements (e.g. electrodes 2145, 2147) mounted on, or formed as part of, an outer surface (e.g. case) of the IPG 2133. As previously described elsewhere, this arrangement may be used to sense cardiac information (e.g. ECG, other), impedance, etc.

In some examples, whether mounted on a single housing (e.g. IPG 2133 in FIG. 60B) or placed in multiple different location (or on different components), the electrode(s) shown in FIG. 60A, 35AA may be used to sense cardiac information (including cardiac morphology), respiratory information (including respiratory morphology), motion/movement of the chest and/or neck, etc., as described in association with sensing portion 2000 (FIG. 58) and/or sensing portion 2510 (FIG. 61A). In some examples, this sensed information may comprise a respiratory rate and/or a heart rate. In some such examples, the sensed respiratory information and/or cardiac information may comprise at least some of substantially the same features and attributes as described in association with respiration portion 2580 and/or cardiac portion 2600 in FIG. 61A.

In some examples, the respiratory information is obtained via measuring trans-thoracic impedance solely via the electrodes on the IPG or via electrodes in addition to those present on the surface of the IPG. However, in some examples, the respiratory information may be derived from the ECG information.

With this in mind, in some examples described elsewhere in the present disclosure, the respiratory information and/or cardiac information may be obtained via an accelerometer (2026 in FIG. 58), which may be located in the IPG 2133. As previously noted, there may be times at which the accelerometer is used to sense respiratory information, cardiac information, and/or other information in order to determine sleep-wake status while at other times, sensing modalities (e.g. ECG electrodes, EMG electrodes, etc.) other than an accelerometer may be used to sense respiration information, cardiac information, and/or other information to determine sleep-wake status.

Unless noted specifically otherwise, it will be understood that the electrodes described in FIG. 60A comprise an exposed electrically conductive portion to engage bodily tissues, etc. within the patient.

In some examples, a single SDB care device comprises a single housing. In some examples, the single device comprises an on-board power source. In some examples, a single device comprises a plurality of sensing elements (e.g. electrodes). In some examples, at least one sensing element (e.g. electrode) is located on two separate portions of a device. For instance, one electrode may be located on IPG 2133 while one electrode may be located on a stimulation lead body 2118.

As previously described in association with at least FIGS. 60A-60B, in some examples an implantable pulse generator (IPG) may take the form of a microstimulator, and may be used to implement various sensing modalities as previously described. At least some example implementations of such a microstimulator are shown in at least FIGS. 60C and 60F.

As shown in FIG. 60C, an example device 2359 including an example microstimulator 2355 may be implanted in a head-and-neck region 2302 of a patient, and in particular in the neck region 2303 in this example. The microstimulator 2355 is implanted subcutaneously via access-incision 2311. In the particular illustrated example, a stimulation electrode 2310 is electrically connected to and extends from the microstimulator 2355, with stimulation electrode 2310 coupled to nerve 2305 to stimulate the nerve, which causes contraction of musculature (e.g. tongue) maintain or restore upper airway patency to treat sleep disordered breathing. In some examples, the stimulation electrode 2301 may comprise at least some of substantially the same features and attributes as stimulation electrode 2112 in FIG. 60A, including acting as a sensing electrode in some examples.

As further shown in the schematic representation of an example device 2400 in FIG. 60D, in some examples the microstimulator 2355 may comprise at least one electrode (e.g. 2402 and/or 2404) relative to which sensing vectors V1, V2, and/or V3 among electrodes 2310, 2402, 2404 may be established to sense physiologic phenomenon (e.g. ECG, bioimpedance, motion at (or of) the neck 2303, etc.) as previously described. This sensed physiologic information may be used to determine a sleep-wake status, among other things, such as implementing stimulation therapy. It will be further understood that in some examples, additional sensing modalities (e.g. EMG) described in association with FIGS. 60A, 24A, and 61A may be implemented via at least a portion of the microstimulation devices of FIGS. 60C-60G. While not fully shown in FIG. 60C, FIG. 60D illustrates that electrode 2310 may be arranged on a lead 2313 extending from microstimulator 2355.

As further shown in the schematic representation of an example device 2420 in FIG. 60E, in some examples the microstimulator 2355 may comprise an accelerometer 2422 and by which sensing physiologic information (e.g. via sensing motion at or of the neck, etc.) may be implemented as previously described throughout the present disclosure. In addition, the microstimulator 2355 in FIG. 60E also may comprise an electrode 2402 (as in FIG. 60D) by which at least some of the previously described sensing (e.g. cardiac, ECG, bioimpedance, motion, etc.) may be implemented via sensing vector V2. This sensed physiologic information may be used to determine a sleep-wake status, among other things, such as implementing stimulation therapy.

FIG. 60F provides a schematic representation 2450 of an example device 2459 which comprises at least some of substantially the same features as the devices of FIGS. 60C-60E, except further comprising a dedicated sensing lead 2433 extending (subcutaneously) into tissue from microstimulator 2355 to support at least one electrode (e.g. 2431, 2432) spaced apart from microstimulator 2355 and/or other electrodes (e.g. 2431, 2432 or 2404 in FIG. 60G). This arrangement may be used to sense physiologic information (e.g. ECG, bioimpedance, motion at or of neck 2303) via vectors V1, V2, V3, V5, V6, and/or V7 (FIG. 60G) in a manner similar to that described for at least example arrangement in FIG. 60C-60E.

In some examples, the microstimulator devices of FIGS. 60C-60G facilitate SDB care, including sleep-wake determination, in a compact arrangement in which sensing, stimulation, implant-access, etc. may be implemented in a single body region (e.g. neck) instead of being dispersed among several body regions (e.g. neck and torso), thereby simplifying implantation and SDB care. For instance, the neck-located microstimulator devices may sense physiologic phenomenon (e.g. respiration, cardiac, etc.) which may sometimes primarily be associated with a different region of the body (e.g. chest) while simultaneously conveniently placing a stimulation element in the neck region in which the microstimulator is located.

FIG. 59 is a block diagram schematically representing an example processing portion 2200, which may form part of and/or be in communication with at least sensing portion 2000 (FIG. 58). In general terms, the processing portion processes signals and/or information obtained by a single sensor, single sensor type, or multiple types of sensors as described in association with at least FIG. 58. As shown in FIG. 58, processing portion 2200 may comprise a filtering function 2210 to filter the sensed signals to exclude noise, non-relevant information, etc. In some examples, the processing portion 2200 may comprise interpretation function 2212, which may interpret the information sensed via sensing portion 2000 in light of sensed physiologic information present in typical sleep patterns. In some such examples, the interpretation may be performed, at least partially with respect to information associated with a reference parameter 2220. In some such examples, the information available via reference parameter 2220 (for interpreting sensed information) may comprise a respiratory rate and/or respiratory signal morphology and/or may comprise a cardiac rate and cardiac signal morphology. Normalization may or may not be utilized.

In some examples, the sensing portion 2000 (FIG. 58) and/or processing portion 2200 (FIG. 59) may be employed in methods to extract important features from sensor signals. Such feature extraction may comprise band-pass filtering, frequency analysis, power spectral analysis, signal amplitude analysis, derivative signal analysis, use of thresholds, and differential signal analysis. Moreover, in some examples, such feature extraction also may comprise amplification and gain control, outlier rejection methods, be based on physiological rates, and/or wavelet analysis, as well as combinations of the preceding parameters. In some examples, the feature extraction may relate to and/or be performed to enable analysis of periods of periodic behavior. For instance, feature extraction may be performed on the sensed signal and analyzing the extracted feature as a moving average or in discrete time chunks as a distribution to determine if the particular extracted feature (e.g. heart rate, heart rate variability, respiratory rate, etc.) has reached a threshold of stability or exhibits a change from the previous behavior.

In some examples, at least a part of processing portion 2200 may comprise, and/or be implemented, at least some of the features and attributes described in association with FIGS. 56-61A.

In some examples, all or a portion of processing portion 2200 may be incorporated within sensing portion 2510 or other portions of care engine 2500 in FIG. 61A and/or may be incorporated within control portion 4000 (FIG. 62A).

FIG. 61A is a block diagram schematically representing an example care engine 2500. In some examples, the care engine 2500 may form part of a control portion 4000, as later described in association with at least FIG. 362A, such as but not limited to comprising at least part of the instructions 4011 and/or information 4012. In some examples, the care engine 2500 may be used to implement at least some of the various example devices and/or example methods of the present disclosure as previously described in association with FIGS. 1A-60G and/or as later described in association with FIGS. 61B-68. In some examples, the care engine 2500 (FIG. 61A) and/or control portion 4000 (FIG. 62A) may form part of, and/or be in communication with, a pulse generator (e.g. 2133 in FIG. 60A-60C) whether such elements comprise a microstimulator or other arrangement.

In one aspect, at least the sensing portion 2510 of care engine 2500 in FIG. 61A directs the sensing of information, and/or receives, tracks, and/or evaluates sensed information obtained via one or more of the sensing modalities, sensing elements, etc. of sensing portion 2000 (FIG. 58B), with care engine 2500 employing such information to determine sleep-wake status, among other actions, functions, etc. as further described below.

As shown in FIG. 61A, in some examples the care engine 2500 comprises a sensing portion 2510, a sleep state portion 2650, a sleep disordered breathing (SDB) parameters portion 2800, and/or a stimulation portion 2900. In some examples, the sensing portion 2510 may comprise an EEG parameter 2512 to sense EEG information, such as a single channel (2514) or multiple channels of EEG signals. Such sensed EEG information may be obtained via EEG sensor 2012 (FIG. 24) or derived from information sensed via another sensing modality. In some examples, the EEG information sensed per parameter 2512 comprises sleep state information. In some such examples, the sleep state information may comprise the parameters provided in the later described sleep state portion 2650 of care engine 2500.

In some examples, the sensing portion 2510 may comprise an electroocoulogram (EOG) parameter 2524, which relates to receiving, tracking, evaluating, and/or directing sensing of eye movement, eye position, etc., such as via an EOG sensor (e.g. 2024 in FIG. 58). In some such examples, the sensing element may comprise an optical sensor.

In some examples, this EOG information may be used as part of determining and/or confirming sleep state information, among other CNS information (2532) which may be used to sense, diagnose, and/or treat sleep disordered breathing (SDB) behavior. For instance, in some such examples, this EOG information may comprise detection and/or tracking of rapid eye movement (REM) per parameter 2668 (FIG. 61A) during sleep, which in turn may be used in differentiating between an awake state, REM state, and/or other sleep states, including various sleep stages.

As further shown in FIG. 36, the care engine 2500 may comprise a sleep state portion 2650 to sense and/or track sleep state information, which may be obtained via the EEG information parameter 2512, in some examples. In some examples, the sleep state portion 2650 may identify and/or track onset (2660) of sleep and/or offset (2662) of sleep, as well as identify and/or track sleep stages once the patient is asleep. Accordingly, in some examples, the sleep state portion 2650 comprises sleep stage parameter 2666 to identify and/or track various sleep stages (e.g. REM and N1, N2, N3 or S1, S2, S3, S4) of the patient during a treatment portion or during longer periods of time. In some instances, the various stages (e.g. N1-N3 or S1-S4) other than REM sleep may sometimes be referred to as non-REM sleep. The sleep state portion 2650 also may comprise, in some examples, a separate rapid eye movement (REM) parameter 2668 to sense and/or track REM information in association with various aspects of sleep disordered breathing (SDB) care, as further described below and throughout various examples of the present disclosure. In some examples, the REM parameter 2668 may form part of, or be used with, the sleep stage parameter 2666.

In some examples, the sleep state portion 2650 may comprise a wakefulness parameter 2664 to direct sensing of, and/or to receive, track, evaluate, etc. sensing a wakeful state of the patient. An awake state of a patient may be indicative of general non-sleep periods (e.g. daytime) and/or of interrupted sleep events, such as macro-arousals (per parameter 2672) associated with a patient waking up to use the restroom (e.g. urinate, etc.), rolling over in bed, waking up in the morning to turn off their alarm, and the like.

Conversely, in some examples, the sleep state portion 2650 may comprise a micro-arousal parameter 2674, by which one may detect and/or track neurological arousals associated with sleep disordered breathing (SDB) events in which a patient experiences a short neurological arousal due to sleep apnea, such as but not limited to obstructive sleep apnea, central sleep apnea, and/or hypopneas. Such SDB-related micro-arousals typically do not result in the patient waking up, in the traditional sense familiar to a lay person. In at least some examples, the stimulation intensity within a treatment period is not varied in response to such SDB-related micro-arousals as one goal of the therapy is for the electrical stimulation to prevent or substantially reduce sleep disordered breathing, which in turn would lessen the frequency and volume of such SDB-related micro-arousals.

In some examples, via at least the sleep state portion 2650 of care engine 2500, the sleep detection method/device may differentiate between wakefulness and sleep disordered breathing (SDB), which occurs during sleep. Among other situations, this differentiation may enable effective neurostimulation therapy such as when a patient is in a sleep position (e.g. laying horizontally or incline position) and the sleep detection arrangement detects a change in sensed data which could possibly be interpreted as a rolling over (e.g. from a supine position onto their side (e.g. lateral decubitus) or vice versa) or as consistent with a SDB behavior. In the case of a bona fide rollover by the patient, such as when getting out of bed, the system will pause the neurostimulation therapy. However, if the detected change may be confirmed as legitimate SDB behavior, then the system/method does not pause the neurostimulation therapy in at least some examples.

With this in mind, in some examples the device/method may differentiate between REM sleep (even where no sleep disordered breathing (SDB) is present) and wakefulness at least because if the patient is in REM sleep, the system avoids pausing neurostimulation therapy for sleep disordered breathing. Conversely, if the patient is in an actual wakeful state, the system should not initiate neurostimulation therapy or may act to pause or to terminate neurostimulation therapy. In some examples, one characteristic feature associated with REM is a lack of body motion, which may sometimes be referred to as paralysis or at least partial paralysis of voluntary muscle control.

In some examples, sleep disordered breathing may occur during REM sleep, such that at least some example device/methods may differentiate sleep disordered breathing from wakefulness and/or differentiate REM sleep from wakefulness. For instance, in some such examples, sensing a lack of body motion may prevent a false positive if/when other parameters (e.g. HR) might otherwise be indicative of wakefulness. For example, during REM sleep stage, sensed information may indicate increased variability in the respiratory period and/or in the heart rate (HR) of the patient.

In some such examples and as previously described, the sleep state information (per sleep state portion 2650) may be used to direct, receive, track, evaluate, diagnose, etc. sleep disordered breathing (SDB) behavior. In some such examples and as previously described, the sleep state information may be used in a closed-loop manner to initiate, terminate, and/or adjust stimulation therapy to treat sleep disordered breathing (SDB) behavior to enhance device efficacy. At least some example closed-loop implementations are further described later in association with at least parameter 2910 in FIG. 61A.

For instance, in some examples via sensing wakefulness (2664 in a sleep state portion 2650), stimulation therapy may be terminated automatically. In some examples, via sensing commencement of particular sleep stages (2666), stimulation therapy may be initiated automatically. In some examples, the intensity of stimulation therapy may be adjusted and implemented according to a particular sleep stage and/or particular characteristics within a sleep stage. In some examples, a lower stimulation intensity level may be implemented upon detecting a REM sleep stage. In some examples, stimulation intensity may be decreased in some sleep stages to conserve power and battery life as well as to improve patient comfort and/or therapy utilization.

In some examples, in cooperation with at least sleep stage parameter 2666 of care engine 2500, delivery of a stimulation signal may be toggled among different predetermined intensity levels for each different sleep stage (e.g. N1, N2, N3 or S1, S2, S3, S4, REM).

In addition to the above described sensing parameters, modalities, etc. described in association with FIG. 61A, in some examples, the sensing portion 2510 of care engine 2500 comprises an ECG parameter 2520, EMG parameter 2522, accelerometer parameter 2526, pressure parameter 2537, temperature parameter 2538, acoustic parameter 2539 to direct sensing of, and/or to receive, track, evaluate, etc. sensing signals from the previously described ECG sensor 2020, EMG sensor 2022, accelerometer 2026, pressure parameter 2037, temperature sensor 2038, and/or acoustic sensor 2039 in association with FIG. 58A. In some examples, the EMG parameter 2522 may comprise detecting muscle activity and/or motion at interscostal muscles, the upper airway, and/or the tongue, such as described in association with at least FIG. 60A and other examples throughout the present disclosure.

In some examples, the sensing portion 2510 of care engine 2500 (FIG. 61A) comprises an impedance parameter 2536 to sense and/or track sensing of impedance within the patient's body to sense motion at (or of) the chest and/or neck and/or other parameters in order to determine sleep-wake status. In addition to or instead of being used to determine sleep-wake status, the impedance parameter 2536 also may be used to sense respiratory information, and/or other information in association with sleep disordered breathing (SDB) care. The impedance parameter 2536 may obtain impedance information from impedance sensor 2036 in FIG. 58 and/or other sensors.

In some examples, sensing portion 2510 of care engine 2500 may comprise a posture parameter 2540 to direct sensing of, and/or to receive, track, evaluate, etc. sensing signals from the previously described posture sensor 2040 in FIG. 58 or other posture, body-position sensor, etc. Like the other parameters of sensing portion 2510, the posture parameter 2540 may be used alone or in combination with other parameters to determine a sleep-wake state of the patient. As previously noted, however, in some example methods (and/or devices) a determination of sleep-wake status may be made without (or independent of) posture information.

In some examples, sensing portion 2510 of care engine 2500 comprises a snoring parameter 2545 to direct sensing of, and/or to receive, track, evaluate, etc. snoring information, which in some examples may be detected and obtained via motion sensing. This sensed snoring information may be used, in some examples, to at least partially determine a sleep-wake status. In one aspect, snoring may be defined as noise associated with each exhalation when respiratory periods are relatively stable and with stable frequency content. Conversely, talking lacks stable respiratory periods and frequency content, and therefore would not be detected as snoring. As noted elsewhere, in some examples the snoring is sensed via acoustic sensor 2039 (FIG. 58) and/or acoustic parameter 2539 (FIG. 61A).

In some examples, sensing portion 2510 of care engine 2500 may comprise a history parameter 2542 by which a history of sensed physiologic information is maintained, and which may be used via comparison parameter 2544 to compare recent sensed physiologic information with older sensed physiologic information. At least some example implementations of using such history parameter 2542 and comparison parameter 2544 are described in association with at least FIGS. 23-24.

In some examples, via care engine 2500, at least some example methods to determine a sleep-wake status may comprise identifying sleep via trends (including variability) in a respiratory rate and/or in a heart rate. In some examples, determination of the sleep-wake status may comprise identifying sleep via a morphology of respiratory cycles, via stability of a respiratory rate, and/or stability in the respiratory morphology. At least some of these examples are further described below in association with at least respiration portion 2580 of care engine 2500.

As shown in FIG. 61A, in some examples, care engine 2500 may comprise a respiration portion 2580. In at least some examples, in general terms respiration portion 2580 may direct sensing of, and/or receive, track, and/or evaluate respiratory morphology, including general patterns and/or specific fiducials within a respiratory signal. In some examples, the respiration portion 2580 may operate in cooperation with, or as part of, sensing portion 2510 of care engine 2500 in FIG. 61A and/or sensing portion 2000 (FIG. 58). At least some aspects of such respiratory morphology managed via respiration portion 2580 may comprise inspiration morphology (parameter 2582) and/or expiration morphology (parameter 2584). In some examples, the respective inspiration morphology parameter 2582 and/or expiration morphology parameter 2584 may comprise amplitude, duration, peak 2586, onset 2588, and/or offset 2590 of the respective inspiratory and/or expiratory phases of the patient's respiratory cycle. In some examples, the detected respiration morphology may comprise transition morphology 2592 such as an inspiration-to-expiration transition and/or an expiration-to-inspiration transition. In some examples, any one or more of these aspects (e.g. peak, onset, offset, magnitude, etc.) of the respective inspiratory and expiratory phases may be used to at least partially determine sleep and/or wakefulness.

For example, the inspiration-to-expiration transition associated with respiration portion 2580 of care engine 2500 may be used as a fiducial to detect and/or track a respiratory rate (and respiratory rate variability), which may be indicative of a change in wake-sleep status. In some examples, changes in a duration of the inspiration-to-expiration transition, changes in peak-to-peak amplitude, and/or changes in the respiratory rate may be indicative of sleep and/or wakefulness, and therefore used to determine a sleep-wake status.

With regard to the sensing, tracking, etc. of respiratory morphologies described above, FIG. 61B is a diagram 150 schematically representing a respiratory cycle 150 which illustrates at least some aspects of respiratory morphology, with respiratory cycle 150 including an inspiratory phase 162 and an expiratory phase 170. The inspiratory phase 162 includes an initial portion 164 (e.g. onset), inspiratory peak 165, end portion 166 (e.g. offset), while expiratory phase 170 includes an initial portion 174 (e.g. onset), intermediate portion 175 (including expiratory peak 177), and end portion 176 (e.g. offset). The above-noted peak parameter 2586, onset parameter 2588, and offset parameter 2590 of the inspiration morphology 2582 (in sensing portion 2510 of care engine 2500) corresponds to the inspiration peak 165, inspiration onset 164, and inspiration offset 166 of the respiratory cycle diagram 150 in FIG. 61B, while the above-noted peak parameter 2586, onset parameter 2588, and offset parameter 2590 of the expiration morphology parameter 2584 (in sensing portion 2510 of care engine 2500) corresponds to the expiratory peak 177, expiratory onset 164, and expiratory offset 166 of the respiratory cycle diagram 150 in FIG. 61B.

In the respiratory cycle diagram 150 in FIG. 61B, a first transition 180 occurs at a junction between the end inspiratory portion 166 and the initial expiratory portion 174. In some instances, this transition 180 may sometimes be referred to as an inspiration-to-expiration transition 180, which as noted above may be used to determine a sleep-wake status per parameter 2592 of respiration portion 2580 of care engine 2500 in FIG. 61A. A second transition 182 occurs at a junction between the end expiratory portion 176 and the initial inspiratory portion 164. In some instances, this transition 182 may sometimes be referred to as an expiration-to-inspiration transition 182, which as noted above may be used to determine a sleep-wake status per parameter 2592 of respiration portion 2580 of care engine 2500 in FIG. 61A.

In some examples, as shown in FIG. 61A the respiration portion 2580 may comprise a chest wall parameter 2594 to direct sensing of and/or receive, track, evaluate, etc. chest wall behavior of the patient. In some such examples, the chest behavior may comprise chest wall motion (e.g. ribcage motion). In some examples, the sensed chest wall motion (e.g. used in determining sleep-wake status) may comprise general motion (e.g. rise and fall) of the chest wall associated with inspiration and expiration of a respiratory cycle as the patient breathes. In some instances, this chest wall motion may comprise intercostal muscle contraction. In some examples, this sensed general chest wall motion (e.g. used in determining sleep-wake status) does not include characteristics such as pectoral muscle contraction and/or signal information (which may be unrelated to breathing and/or cardiac function). Among other uses, the sensed chest motion may be used to determine respiratory information, cardiac information and/or other physiologic information in order to determine a sleep-wake status, as further described throughout various examples of the present disclosure. For instance, one use of the sensed chest motion is to at least partially determine whether respiration is passive or active (e.g. forced), which in turn may be used to determine a sleep-wake state. As just one example aspect of passive respiration, normal exhalation occurs without direct muscular effort, as during normal tidal breathing when air may be expelled from the lungs as a result of the recoil effect of elastic tissues in the chest, lungs, and diaphragm. This behavior would be expected in a sleep state. In contrast, one example of active respiration, which may be associated with an awake state, includes forced exhalation which involves contraction of the abdominal wall, internal intercostal muscles, and diaphragm.

In some examples, as shown in FIG. 61AS the respiration portion 2580 may comprise a neck parameter 2595 to direct sensing of and/or receive, track, evaluate, etc. neck movement of the patient, which may be indicative of respiratory information and/or cardiac information regarding the patient, which may be used to determine a sleep-wake state. As previously described, such sensed movement of the neck and/or at the neck may comprise movement such as (but not limited to) motion from the airway and/or blood vessels, impedance, and/or other physiologic phenomenon. For instance, at least some sensed impedance vectors may be measured across the airway, across a vessel, and/or across both.

In some examples, the respiratory portion 2580 may comprise a respiratory rate parameter 2596 to direct sensing of, and/or receive, track, evaluate, etc. respiratory rate information including a respiratory rate, respiratory rate variability 2597, etc., which may be used to determine a sleep-wake status or change in sleep-wake status. In some examples, sensing the respiratory rate (and any associated variability, trends, etc.) may be implemented via sensing and tracking one of the above-noted identifiable parameters (e.g. peak, onset, offset, transition) of respiration morphology per respiratory portion 2580 of care engine 2500.

As shown in FIG. 61A, in some examples the care engine 2500 may comprise a cardiac portion 2600. In some examples, in general terms the cardiac portion 2600 may be employed to sense, track, determine, etc. cardiac information, which may be indicative of a sleep-wake status, among other information pertinent to SDB care. In some examples, the cardiac portion 2600 may operate in cooperation with, or as part of, sensing portion 2510 of care engine 2500 (FIG. 61A) and/or sensing portion 2000 (FIG. 58A). The cardiac portion 2600 may be employed, alone or in combination with, other elements, modalities, etc. of the care engine 2500. In some examples, the cardiac portion 2600 may employ a single type of sensing or multiple types of sensing in sensing portion 2510, and in some examples, the cardiac portion 2600 may employ other sensing types, modalities, etc. in addition to, or as an alternative to, the particular sensing types, modalities of sensing portion 2510. Moreover, the cardiac portion 2600 may determine, track, etc. a sleep-wake status in cooperation with, or independent of, the respiration portion 2580 of care engine 2500.

In some examples, in general terms the cardiac portion 2600 may direct sensing of, and/or receive, track, evaluate, etc. cardiac signal morphology to at least determine a sleep-wake status. As shown in FIG. 61A, in some examples the cardiac portion 2600 comprises an atrial morphology parameter 2610 and/or a ventricular morphology parameter 2612, which may be employed alone, or in combination, to determine a sleep-wake status. In some examples, at least some aspects of the respective atrial and ventricular morphologies 2610, 2612 may comprise detecting contraction (parameter 2620) and/or relaxation (parameter 2622) of the atria and ventricles, respectively. In some such examples, the tracking of the respective contraction and/or relaxation may facilitate determining a sleep-wake status by providing a readily identifiable portion of a cardiac waveform by which heart rate (HR) and/or heart rate variability (HRV) may be detected, tracked, and from which values, trends, etc. of the heart rate or heart rate variability may indicate sleep or wakefulness.

In some examples, at least some aspects of the respective atrial and ventricular morphologies 2610, 2612 may comprise a peak 2630 of an atrial or ventricular contraction, which may be used to determine a sleep-wake status.

In some examples, at least some aspects of the respective atrial and ventricular morphologies 2610, 2612 may comprise an onset (e.g. start) 2632 of an atrial contraction, of an atrial relaxation, of a ventricular contraction, or of a ventricular relaxation. In some examples, at least some aspects of the respective atrial and ventricular morphologies 2610, 2612 may comprise an offset (e.g. termination, end) 2634 of an atrial contraction, an atrial relaxation, a ventricular contraction, or ventricular relaxation.

In some examples, at least some aspects of the respective atrial and ventricular morphologies 2610, 2612 by which a sleep-wake status may be detected may comprise a combination of atrial and ventricular contraction.

In some examples, at least some aspects of the respective atrial and ventricular morphologies 2610, 2612 may comprise a transition 2640, such as a transition between different phases of the cardiac cycle.

In some examples, at least some aspects of the cardiac information (by which a sleep-wake status may be determined) may comprise opening or closing of a heart valve per parameter 2642. In some examples, such detection of opening and/or closing of a heart valve (per parameter 2642) also may be used to help determine the timing and/or occurrence of an onset and/or offset of a contraction (or relaxation) of an atria or ventricles in association with parameters 2610, 2612, 2620, 2622, 2630, 2632, 2634.

In some examples, cardiac information may comprise heart motion 2644, from which the above-described cardiac morphology parameters may be determined. The heart motion 2644 may be obtained via one or more of the various sensing modalities (e.g. accelerometer, EMG, etc.) described in association with at least FIG. 58.

As further shown in FIG. 61A, in some examples, cardiac information may comprise heart rate parameter 2645 to direct sensing of, and/or receive, track, evaluate, etc. heart rate information including a heart rate (HR), heart rate variability (HRV) 2646, etc., which may be used to determine a sleep-wake status or change in sleep-wake status. In some examples, sensing the heart rate (and any associated variability, trends, etc.) may be implemented via sensing and tracking one of the above-noted identifiable parameters (e.g. peak, onset, offset, transition) of cardiac morphology per cardiac portion 2600.

In some examples, at least some of the above-described cardiac information may be determined, at least partially, according to heart sounds (e.g. S1, S2, etc.), which may be sensed acoustically (e.g. 2039 in FIG. 24; 2539 in FIG. 61A).

In some examples, sleep-wake status may be determined via a combination of sensed respiratory features and sensed cardiac features. At least some aspects of use of this combination of information are previously described in association with at least FIGS. 142-48B, and elsewhere throughout examples of the present disclosure.

As further shown in FIG. 61A, in some examples the care engine 2500 comprises a SDB parameters portion 2800 to direct sensing of, and/or receive, track, evaluate, etc. parameters particularly associated with sleep disordered breathing (SDB) care. For instance, in some examples, the SDB parameters portion 2800 may comprise a sleep quality portion 2810 to sense and/or track sleep quality of the patient in particular relation to the sleep disordered breathing behavior of the patient. Accordingly, in some examples the sleep quality portion 2810 comprises an arousals parameter 2812 to sense and/or track arousals caused by sleep disordered breathing (SDB) events with the number, frequency, duration, etc. of such arousals being indicative of sleep quality (or lack thereof). In some such examples, such arousals may correspond to micro-arousals as described in association with at least parameter 2674 in sleep state portion 2650 of care engine 2500 in FIG. 61A.

In some examples, the sleep quality portion 2810 comprises a state parameter 2814 to sense and/or track the occurrence of various sleep states (including sleep stages) of a patient during a treatment period or over a longer period of time. In some such examples, the state parameter 2814 may cooperate with, form part of, and/or comprise at least some of substantially the same features and attributes as sleep state portion 2650 of care engine 2500.

In some examples, the SDB parameters portion 2800 comprises an AHI parameter 2830 to sense and/or track apnea-hypopnea index (AHI) information, which may be indicative of the patient's sleep quality. In some examples, AHI information is sensed throughout each of the different sleep stages experienced by a patient, with such sensed AHI information being at least partially indicative of a degree of sleep disordered breathing (SDB) behavior. In some examples, the AHI information is obtained via a sensing element, such as one or more of the various sensing types, modalities, etc. in association with at least sensing portion 2000 (FIG. 24) and/or sensing portion 2510 (FIG. 27A), which may be implemented as described in various examples of the present disclosure. In some examples, AHI information may be sensed via a sensing element, such as an accelerometer located in either the torso or chin/neck region with the sensing element locatable and implemented as described in various examples of the present disclosure. In some examples, a combination of accelerometer-based sensing and other types of sensing may be employed to sense and/or track AHI information. In some examples, the AHI information is obtained via sensing modalities (e.g. ECG, impedance, EMG, etc.) other than via an accelerometer.

In some examples, determination of sleep-wake status may be implemented via a probability portion 3200 of care engine 2500 in FIG. 61A. In some such examples, via a selection parameter 3210, the probability portion 3200 may enable selective inclusion or selective exclusion of at least some sleep-wake determination parameters without directly affecting the general operation of determining sleep-wake status. In some examples, via the probability function 3200, a sensitivity parameter 3220 may be adjusted by a patient, clinician, caregiver to increase or decrease a sensitivity of determining the sleep-wake status via a particular parameter. In some examples, a machine-learning parameter 3230 may be implemented to assess and modify adjustments to probabilistic determinations of the sleep-wake status, including but not limited to, adjustments to any amplitude thresholds, duration thresholds, etc. associated with a probabilistic determination of sleep-wake status. In some examples, employing such probabilistic determinations may permit more granular controls of a patient's individual signals (used in combination to make the determination of sleep-wake status), which in turn, may enable balancing simple control with the capability of complex control and sensor flexibility when desired.

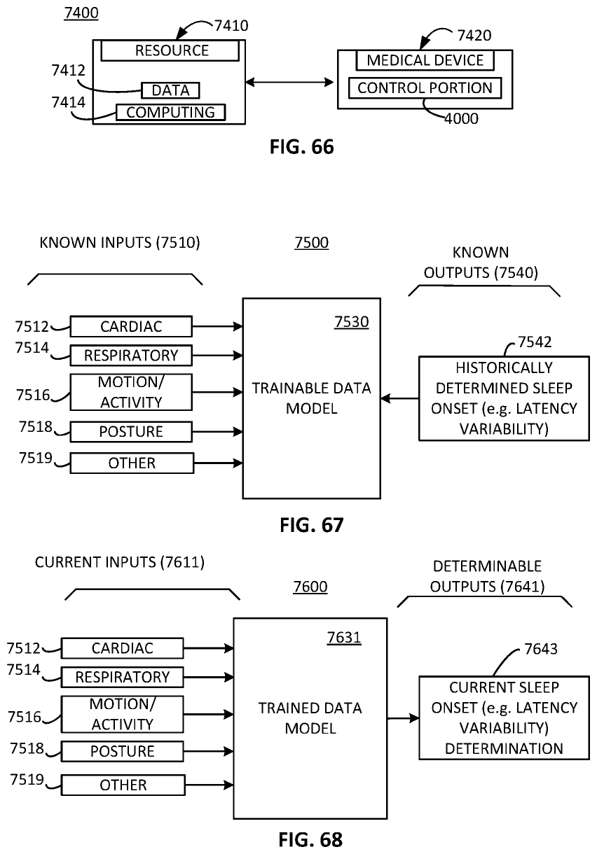

In some examples, via at least the machine-learning parameter 3230, the care engine 2500 may comprise and/or access a neural network resource (e.g. deep learning, convolutional neural networks, etc.) to identify patterns indicative of sleep from a single sensor of multiple sensors. In some examples, a decision tree-based expert resource also could be used to combine sensors or neural network output with other signals such as time of day or remote inputs/usage. One example implementation of machine learning, such as via parameter 3230, is further described later in association with at least FIGS. 66-68. At least some other example implementations are described throughout the present disclosure.

In some examples, via probability portion 3200, care engine 2500 may assign and apply a weight (parameter 3240) to be associated with each signal in order to increase (or decrease) the relative importance of a particular sensor signal in determining sleep-wake status.

In some examples, via a temporal emphasis parameter 3250 different thresholds may be selected for different times of a 24 hour daily period. For instance, during a first period (e.g. daytime such as Noon) some parameters may be de-emphasized and/or other parameters emphasized, while during a second period (e.g. late evening such as 10 pm), some parameters may be emphasized in determining sleep-wake status while other parameters are de-emphasized. Alternatively, during the first period, the sensitivity of most or all parameters (for determining sleep-wake status) may be decreased and during the second period, the sensitivity of some or all parameters (for determining sleep-wake status) may be increased.

In some such examples, this adjustability via the temporal emphasis parameter 3250 may enhance sleep-wake determinations for a patient having non-standard sleep periods, such as a graveyard shift worker (e.g. works 11 pm-7 am), because their intended sleep period (e.g. 8 am-3 pm) conflicts with a conventional sleep period (e.g. 10 pm-6 am).

In some examples, the probability function 3200 of care engine 2500 may implement a probabilistic determination of sleep-wake status based on sensing motion at (or of) the chest, neck, and/or head. In some such examples, an accelerometer and/or other sensors (e.g. impedance, EMG, etc.) may be employed to sense motion at (or of) the chest, neck, and/or head. In some such examples, per a differentiation parameter 3260, where sensing is performed via a sensor (e.g. accelerometer) with multiple signal components (e.g. a multiple axis accelerometer) or captures a signal (e.g. ECG) from which multiple different signals may be derived, an example method may comprise dividing a signal associated with sensing the physiologic information into a plurality of different signals with each respective signal representing a different sleep-wake determination parameter. Stated differently, multiple components within a signal are differentiated into distinct and separate signals, each of which may be indicative of sleep-wake status. A probability of sleep-wake status is then determined based on assessing the respective different signals associated with the respective different sleep-wake determination parameters. As noted above, in some examples each respective different signal may comprise one axis of a multiple axis accelerometer (e.g. in which each axis is orthogonal to other axes) or may comprise a single axis accelerometer (when multiple single-axis accelerometers are employed). In some such examples, a different processing method or technique may be applied to at least some of the signal components (e.g. sleep determination parameters).

As shown in FIG. 61A, in some examples the care engine 2500 may comprise an activation portion 3000, which in general terms may control activation of an implantable medical device, such as an IPG. In some such examples, nerve stimulation delivery via the implantable medical device may be activated and terminated automatically 3010, with such activation and termination based on sleep-wake status. In some such examples, the sleep-wake status is determined automatically via care engine 2500.

Accordingly, in some examples, via automatic parameter 3010, at least some example methods and/or devices for determining sleep-wake status may be used to automatically initiate a treatment period (e.g. upon automatically detecting sleep) and to automatically terminate a treatment period (e.g. upon automatically detecting wakefulness).

In some examples in which the automatic determination of a sleep-wake status is unavailable or deactivated by the patient (or clinician or caregiver), then per remote parameter 3012 the treatment period may comprise a period of time beginning with the patient using a remote control to turn on the therapy device and ending with the patient turning off the device via the remote control. In some examples, per remote parameter 3012, a treatment period may be initiated and/or terminated based on at least one a degree of ambient light sensed via a remote control, a degree or type of motion sensed by the remote control, and/or the above-described therapy activation (e.g. on, off) implemented via the remote control. In some examples, the remote control may comprise the remote control 4340 shown in FIG. 64. It will be understood that, in some examples, detecting the degree of ambient light and/or the degree or type of motion of the remote control may be used as part of other features described herein to perform an automatic determination of sleep-wake status, which in turn may determine automatic initiation, termination, pause, adjustment, etc. of a treatment period in which neurostimulation therapy is applied. In some examples, remote control parameter 4340 may be implemented in association with method 788 in FIG. 53I.

In some examples in which the automatic determination of a sleep-wake status is unavailable or deactivated by the patient (or clinician or caregiver), then per app parameter 3013 the treatment period may comprise a period of time beginning with the patient using an app to turn on the therapy device and ending with the patient turning off the device via the app. In some examples, per app parameter 3013, a treatment period may be initiated and/or terminated based on at least one a degree of ambient light sensed via app, a degree or type of motion sensed by the mobile device, and/or the above-described therapy activation (e.g. on, off) implemented via the app on the mobile device. In some examples, the app may comprise the app 4330 shown in FIG. 64, which may be implemented via a mobile device 4320 (FIG. 64), such as a mobile smart phone, tablet, phablet, smart watch, etc. The mobile device may comprise a control portion, user interface (e.g. display) operate the app, and the mobile device may comprise sensor(s) to sense the above-described features (e.g. motion, ambient light, sounds, etc.) in manner to enable the app to perform sleep-wake determination at least partially based on the use (or non-use) of the mobile device.

In some examples, the sensor(s) of a remote control and/or mobile device may comprise an accelerometer, gyroscope, and/or other motion detector.

However, in some examples where automatic determination of sleep-wake status is unavailable (or deactivated), via the temporal parameter 3014 the treatment period may begin automatically at a selectable, predetermined start time (e.g. 10 p.m.) and may terminate at a selectable, predetermined stop time (e.g. 6 a.m.)

In one aspect, the treatment period corresponds to a period during which a patient is sleeping such that the stimulation of the upper airway patency-related nerve and/or central sleep apnea-related nerve is generally not perceived by the patient and so that the stimulation coincides with the patient behavior (e.g. sleeping) during which the sleep disordered breathing behavior (e.g. central or obstructive sleep apnea) would be expected to occur. Accordingly, to avoid enabling stimulation prior to the patient falling asleep, in some examples stimulation can be enabled during the treatment period after expiration of a timer started upon the automatic sleep detection. To avoid continuing stimulation after the patient wakes, stimulation can be disabled upon the automatic detection of wakefulness. Accordingly, in at least some examples, these periods may be considered to be outside of the treatment period or may be considered as a startup portion and wind down portion, respectively, of a treatment period.

In some examples, via a boundary parameter 3016 a selectable, predetermined first time marker (e.g. 10 pm) may be used as a limit or boundary to prevent automatic initiation of a treatment period (based on automatic detection of sleep) before the first time marker, and a selectable, predetermined second time marker (e.g. 6 am) may be used as a limit or boundary to ensure automatic termination of a treatment period to prevent continuance of a treatment period after the second time marker. Via such example arrangements, the treatment period may be initiated automatically via automatic sleep detection and/or may be terminated automatically via automatic wakefulness detection, while providing assurance to the patient of a treatment period not being initiated during normally wakeful periods, or not extending beyond their normal sleep period.

In some examples, determining sleep-wake status in association with boundary parameter 3016 may comprise and/or be combined with at least the features and attributes as previously described in association with method 780 in FIG. 53A, as well as in association with temperature parameter 2038 (FIG. 58) as previously described.

However, in some instances, via a physical parameter 3018 a user may take physical steps to cause activation (or deactivation) of a treatment period for the implantable medical device. For instance, via the activation portion 3000 and physical parameter 3018, the care engine 2500 may receive physical input such as tapping of the chest (or neck or head) or tapping over the implant to activate or deactivate the device. Alternatively, a user may use a patient remote control function 3012 to activate or deactivate the implantable medical device, which in turn may activate or deactivate delivery of nerve stimulation. In some such examples, activation or deactivation of the treatment period (in which nerve stimulation is applied) may be implemented via physical motion of a remote control or a mobile device (e.g. hosting an app). In some instances, via a clinician programmer or remote control, this physical feature (3018) may be activated or deactivated at the discretion of the clinician or user.

As further shown in FIG. 61A, in some examples care engine 2500 comprises a patient eligibility portion 3022 to track, evaluate, and/or control determining sleep onset (including variability of onset latency) and/or use of such information for determining patient eligibility of automatic sleep detection, which in some examples may be used to automatically initiate, pause, and/or terminate therapy (e.g. SDB stimulation therapy) in accordance with various examples of the present disclosure in association with at least FIGS. 1A-66. In some such examples, the patient eligibility portion 3022 may operate in complementary relation with at least the activation portion 3000 (and/or other features, functions, portions, etc. of care engine 2500) to implement the functions, attributes, etc. associating with the various examples of the present disclosure relating to automatic activation (and/or pause, termination), sleep onset determination, sleep onset latency variability determinations, and/or related patient eligibility.

As further shown in FIG. 61A, in some examples care engine 2500 comprises a stimulation portion 2900 to control stimulation of target tissues, such as but not limited to an upper airway patency nerve, to treat sleep disordered breathing (SDB) behavior. In some examples, the stimulation portion 2900 comprises a closed loop parameter 2910 to deliver stimulation therapy in a closed loop manner such that the delivered stimulation is in response to and/or based on sensed patient physiologic information.

In some examples, the closed loop parameter 2910 may be implemented as using the sensed information to control the particular timing of the stimulation according to respiratory information, in which the stimulation pulses are triggered by or synchronized with specific portions (e.g. inspiratory phase) of the patient's respiratory cycle(s). In some such examples and as previously described, this respiratory information may be determined via a single type of sensing or multiple types of sensing via sensing portion 2000 (FIG. 58) and sensing portion 2510 (FIG. 61A).

In some examples in which the sensed physiologic information enables determining (at least) a sleep-wake state, the closed loop parameter 2910 may be implemented to initiate, maintain, pause, adjust, and/or terminate stimulation therapy based on the determined sleep-wake state (including particular sleep stages).

As further shown in FIG. 61A, in some examples the stimulation portion 2900 comprises an open loop parameter 2925 by which stimulation therapy is applied without a feedback loop of sensed physiologic information. In some such examples, in an open loop mode the stimulation therapy is applied during a treatment period without (e.g. independent of) information sensed regarding the patient's sleep quality, sleep state, respiratory phase, AHI, etc. In some such examples, in an open loop mode the stimulation therapy is applied during a treatment period without (i.e. independent of) particular knowledge of the patient's respiratory cycle information.

However, in some such examples, some sensory feedback may be utilized to determine, in general, whether the patient should receive stimulation based on a severity of sleep apnea behavior.

As further shown in FIG. 61A, in some examples the stimulation portion 2900 comprises an auto-titration parameter 2920 by which an intensity of stimulation therapy can be automatically titrated (i.e. adjusted) to be more intense (e.g. higher amplitude, greater frequency, and/or greater pulse width) or to be less intense (e.g. lower amplitude, lower frequency, and/or lower pulse width) within a treatment period.

In some such examples and as previously described, such auto-titration may be implemented based on sleep quality and/or sleep state information, which may be obtained via sensed physiologic information, in some examples. It will be understood that such examples may be employed with synchronizing stimulation to sensed respiratory information (i.e. closed loop stimulation) or may be employed without synchronizing stimulation to sensed respiratory information (i.e. open loop stimulation).

In some examples, at least some aspects of the auto-titration parameter 2920 may comprise, and/or may be implemented, via at least some of substantially the same features and attributes as described in Christopherson et al., SYSTEM FOR TREATING SLEEP DISORDERED BREATHING, issued as U.S. Pat. No. 8,938,299 on Jan. 20, 2015, and which is hereby incorporated by reference in its entirety.

With regard to the various examples of the present disclosure, in some examples, delivering stimulation to an upper airway patency nerve is to cause contraction of upper airway patency-related muscles. In some such examples, the contraction comprises a suprathreshold stimulation, which is in contrast to a subthreshold stimulation (e.g. mere tone) of such muscles. In one aspect, a suprathreshold intensity level corresponds to a stimulation energy greater than the nerve excitation threshold, such that the suprathreshold stimulation may provide for maximum upper-airway clearance (i.e. patency) and obstructive sleep apnea therapy efficacy.

In some examples, at least some example methods may comprise identifying, maintaining, and/or optimizing a target stimulation intensity (e.g. therapy level) without intentionally identifying a stimulation discomfort threshold at the time of implantation or at a later point in time after implantation.

In some examples, upon determining sleep according to a minimum predetermined confidence level, an amplitude (e.g. intensity) of the stimulation signal may start a lower value and then be increased to higher values in a ramped manner. In some such examples, the increases in amplitude (up to a desired/target value) may be made dependent on additional or further predetermined confidence levels. However, if it is later determined that sleep is not occurring but rather that the patient is in a quiet, restful awake state, then the stimulation may be terminated or ramped down while still in the ramping phase, prior to reaching a target stimulation amplitude. Among other applications, this example method may be beneficial for patients with cardiac or respiratory disorders at least because the cardiac morphologies and/or respiratory morphologies (from which sleep may be detected) may be more complex such that accurate detection of actual sleep may be more challenging in such patients.

As noted above in association with the boundary parameter 3016 of the activation portion 3000, a clock or time keeping element within an implantable medical device (e.g. IPG 2133) may be used to implement boundaries or limit for when stimulation therapy (within a treatment period) may be automatically initiated or terminated via automatic sleep detection (or wake detection) per determining a sleep-wake status. In some examples, the time-based boundaries may be based on patient behaviors and/or direct clinician programming. In some examples, such tracked patient behavior may be used as input to a probabilistic model of determining a sleep-wake status. In some examples, the time-based boundaries also may be based, at least in part, on a history of patient activities.

In some examples, the time-based boundaries may account for daylight savings time and travel (e.g. different time zones), and may be adjusted via a patient remote control or physical tapping on the chest. In some such examples, a time-based boundary parameter may comprise one of multiple inputs used to determine sleep-wake status, and which may increase reliability in determining sleep-wake status in a variety of environments (rather than a single time-place environment such as only a patient bedroom).

In some examples, boundary parameter 3016 of activation portion 3000 in FIG. 61A may comprise criteria which are not strictly time-based (e.g. time of day). For instance, in some examples the boundary parameter 3016 may be implemented based on a number, type, and/or duration of various sleep stages associated with a single treatment period (e.g. a night's sleep). For instance, an example method may determine a boundary or an end limit of a treatment period according to observing a certain number (e.g. 4 or 5) of REM sleep periods, stage 4 sleep periods, or stage 3 sleep periods, etc. In some such examples, the number of particular sleep stage periods may be selectable. In some examples, the boundary may be based on a selectable percentage that a patient spends in one or more particular sleep stages.

In some examples, upon detecting a sleep state (per a sleep-wake status) a neurostimulation signal may be applied to a phrenic nerve, in order to treat central sleep apnea. In some examples, determining a sleep-wake status may be used to control initiation and/or termination of stimulation of both an upper airway patency nerve (e.g. hypoglossal nerve) and a diaphragm control nerve (in a manner coordinated relative to each other) to treat sleep disordered breathing.

In some examples, the stimulation portion 2900 may operate cooperatively with at least the respiration portion 2580 and/or the sensing portion 2510 of care engine 2500 (e.g. such as in association with sensing portion 2000 in FIG. 58) to determine efficacy of stimulation, and/or whether a flow limitation exists, by evaluating a flow response within a single respiratory cycle. Such evaluation stands in contrast to performing such evaluation on a cycle-to-cycle basis, such as looking at the respiratory signal from a peak of an inspiratory phase of one cycle to a peak of an inspiratory phase of another cycle.

For instance, in the example method, if the stimulation portion 2900 were to cause a change in the stimulation intensity level (e.g. increase or decrease) during the inspiratory phase, one feature of the care engine 2500 may comprise determining whether a substantial change (e.g. 10%, 15%, 20%, or more) in the flow response occurred.

In some such examples of stimulation portion 2900 in evaluating whether stimulation therapy is efficacious (based on a flow response of the inspiratory phase within a single respiratory cycle versus from cycle-to-cycle), some example methods may comprise determining whether a change (e.g. a substantial change) in the flow response were to occur upon a complete termination of stimulation or upon initiation of stimulation (such as when no stimulation was previously occurring) during an inspiratory phase of a single respiratory cycle.

In some examples, care engine 2500 in FIG. 61A may comprise an initial use function 3100, which in some examples may automatically enhance determination of sleep-wake status. In some such examples, via initial use function 3100 a method and/or device for SDB care may omit a manual training period and instead automatically “normalize” use of the method and/or device for a particular patient. For instance, in some examples, determination of a sleep-wake status may begin with default parameters or may begin with parameters collected at the time of implant of a SDB care device in the patient. In some examples, determination of a sleep-wake status may be performed initially with no default parameters. In some such examples, when wakefulness is detected, sensing portion 2000 (FIG. 58) and/or care engine 2500 (FIG. 61A) may collect respiratory information, motion information, and/or posture information, etc. associated with wakefulness, which in turn may to allow for more sensitive detection of sleep in determining a sleep-wake status. In some examples, detection of wakefulness may comprise detecting gross body motion, such as but not limited to walking, swallowing, torso motion, etc. In some examples, a gravity vector is established at the time of implanting the SDB care device.

With this in mind, per the initial use function 3100, such automatic normalization may comprise omitting the use of absolute thresholds and instead perform determination of sleep-wake status (e.g. detection of onset of sleep) on the basis of percentage change in sensed values. Moreover, in some examples, sensing of various physiologic phenomenon (e.g. respiration, cardiac, etc.) may be used to determine a highest value or lowest value of such physiologic phenomenon and then use such end-of-the-range values to adjust thresholds accordingly.

It will be understood that the various parameters, functions, portions, etc. shown and described in association with FIG. 61A are not limited to the particular groupings, relationships, etc. shown in FIG. 61A, but may be arranged in groupings, relationships, etc. other than shown in FIG. 61A. Moreover, it will be understood that the care engine 2500 (or portions thereof) in FIG. 61A may be implemented with just some (i.e. not all) of the portions, elements, parameters, etc. shown in FIG. 61A.

With reference to at least care engine 2500 in FIG. 61A and the example methods and/or devices described throughout the present disclosure, it will be understood that such engines, methods, and/or devices (and components, portions, etc. thereof) for determining a sleep-wake status also may be used for quantifying activity levels and assessing related health parameters.

FIG. 62A is a block diagram schematically representing an example control portion 4000. In some examples, control portion 4000 provides one example implementation of a control portion forming a part of, implementing, and/or generally managing stimulation elements, power/control elements (e.g. pulse generators, microstimulators), sensors, and related elements, devices, user interfaces, instructions, information, engines, elements, functions, actions, and/or methods, as described throughout examples of the present disclosure in association with FIGS. 1-61B and 62A-68.

In some examples, control portion 4000 includes a controller 4002 and a memory 4010. In general terms, controller 4002 of control portion 4000 comprises at least one processor 4004 and associated memories. The controller 4002 is electrically couplable to, and in communication with, memory 4010 to generate control signals to direct operation of at least some of the stimulation elements, power/control elements (e.g. pulse generators, microstimulators) sensors, and related elements, devices, user interfaces, instructions, information, engines, elements, functions, actions, and/or methods, as described throughout examples of the present disclosure. In some examples, these generated control signals include, but are not limited to, employing instructions 4011 and/or information 4012 stored in memory 4010 to at least determining sleep-wake status of a patient, including particular sleep stages. Such sleep-wake determination may comprise part of directing and managing treatment of sleep disordered breathing such as obstructive sleep apnea, hypopnea, and/or central sleep apnea, with such sleep-wake determination also comprising sensing physiologic information including but not limited to electrical brain activity, respiratory information, heart rate, and/or monitoring sleep disordered breathing, etc. as described throughout the examples of the present disclosure in association with FIGS. 1-61B and 62A-68-. In some instances, the controller 4002 or control portion 4000 may sometimes be referred to as being programmed to perform the above-identified actions, functions, etc. such that the controller 4002, control portion 4000 and any associated processors may sometimes be referred to as being a special purpose computer, control portion, controller, or processor. In some examples, at least some of the stored instructions 4011 are implemented as, or may be referred to as, a care engine, a sensing engine, monitoring engine, and/or treatment engine. In some examples, at least some of the stored instructions 4011 and/or information 4012 may form at least part of, and/or, may be referred to as a care engine, sensing engine, monitoring engine, and/or treatment engine.

In response to or based upon commands received via a user interface (e.g. user interface 4040 in FIG. 63) and/or via machine readable instructions, controller 4002 generates control signals as described above in accordance with at least some of the examples of the present disclosure. In some examples, controller 4002 is embodied in a general purpose computing device while in some examples, controller 4002 is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of this application, in reference to the controller 4002, the term “processor” shall mean a presently developed or future developed processor (or processing resources) that executes machine readable instructions contained in a memory. In some examples, execution of the machine readable instructions, such as those provided via memory 4010 of control portion 4000 cause the processor to perform the above-identified actions, such as operating controller 4002 to implement the sensing, monitoring, determining, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g. non-transitory tangible medium or non-volatile tangible medium), as represented by memory 4010. In some examples, the machine readable instructions may comprise a sequence of instructions, a processor-executable machine learning model, or the like. In some examples, memory 4010 comprises a computer readable tangible medium providing non-volatile storage of the machine readable instructions executable by a process of controller 4002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 4002 may be embodied as part of at least one application-specific integrated circuit (ASIC), at least one field-programmable gate array (FPGA), and/or the like. In at least some examples, the controller 4002 is not limited to any specific combination of hardware circuitry and machine readable instructions, nor limited to any particular source for the machine readable instructions executed by the controller 3002.

In some examples, control portion 4000 may be entirely implemented within or by a stand-alone device.

In some examples, the control portion 4000 may be partially implemented in one of the sensing devices, monitoring devices, stimulation devices, apnea treatment devices (or portions thereof), etc. and partially implemented in a computing resource separate from, and independent of, the apnea treatment devices (or portions thereof) but in communication with the apnea treatment devices (or portions thereof). For instance, in some examples control portion 4000 may be implemented via a server accessible via the cloud and/or other network pathways. In some examples, the control portion 4000 may be distributed or apportioned among multiple devices or resources such as among a server, an apnea treatment device (or portion thereof), and/or a user interface.

In some examples, control portion 4000 includes, and/or is in communication with, a user interface 4040 as shown in FIG. 63.

FIG. 62B is a diagram schematically illustrating at least some example implementations of a control portion 4020 by which the control portion 4000 (FIG. 62A) can be implemented, according to one example of the present disclosure. In some examples, control portion 4020 is entirely implemented within or by a pulse generator (PG) assembly 4025, which has at least some of substantially the same features and attributes as a pulse generator (e.g. power/control element, microstimulator) as previously described throughout the present disclosure. In some examples, control portion 4020 is entirely implemented within or by a remote control 4030 (e.g. a programmer) external to the patient's body, such as a patient control 4032 and/or a physician control 4034. In some examples, the control portion 4000 is partially implemented in the IPG assembly 4025 and partially implemented in the remote control 4030 (at least one of patient control 4032 and physician control 4034).

FIG. 63 is a block diagram schematically representing user interface 4040, according to one example of the present disclosure. In some examples, user interface 4040 forms part or and/or is accessible via a device external to the patient and by which the therapy system may be at least partially controlled and/or monitored. The external device which hosts user interface 4040 may be a patient remote (e.g. 4032 in FIG. 62B), a physician remote (e.g. 4034 in FIG. 62B) and/or a clinician portal. In some examples, user interface 4040 comprises a user interface or other display that provides for the simultaneous display, activation, and/or operation of at least some of the stimulation elements, power/control elements (e.g. pulse generators, microstimulators), sensors, and related elements, devices, user interfaces, instructions, information, engines, functions, actions, and/or method, etc., as described in association with FIGS. 1-68. In some examples, at least some portions or aspects of the user interface 4040 are provided via a graphical user interface (GUI), and may comprise a display 4044 and input 4042.

FIG. 64 is a block diagram 4300 which schematically represents some example implementations by which a device (MD) 4310 (e.g. implantable in some example), such as a pulse generator and/or sensing monitor, may communicate wirelessly with other devices. In some examples, the other devices may be located outside the patient. As shown in FIG. 64, in some examples, the MD 4310 may communicate with at least one of patient app 4330 on a mobile device 4320, a patient remote control 4340, a clinician programmer 4350, and a patient management tool 4336. The patient management tool 4336 may be implemented via a cloud-based portal 4362, the patient app 4330, and/or the patient remote control 4340. Among other types of data, these communication arrangements enable the MD 4310 to communicate, display, manage, etc. sleep/wake data for patient management as well as to allow for adjustment to the detection method if/where needed.

It will be understood that at least some of the various devices/elements 4320, 4340, 4350, patient management tool 4360 also may communicate with each other, with or without communicating with the implantable device 4310.

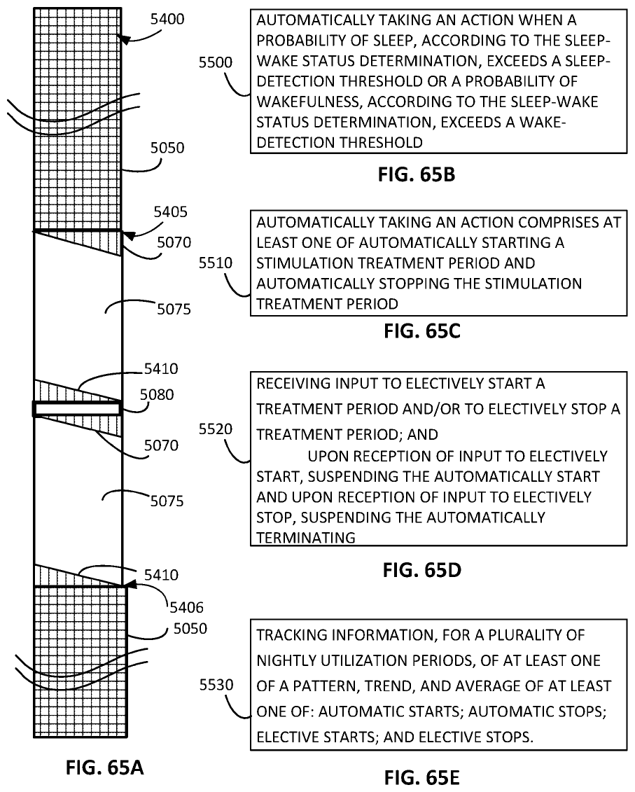

As shown in FIG. 65A, in some examples the user interface 4040 of FIG. 63 also may display and/or report the use of ramped initiation, ramped transitions in and out of a pause in therapy, and/or a ramped termination of stimulation for a given treatment period. For example, as shown in the schematic representation in FIG. 65A, a daily display portion 5400 may comprise various graphic identifiers, such as a wakefulness period 5050, automatic start (e.g. auto-start) instances 5070, on period 5075, etc.

In some example methods, at least some of the start, stop, pauses of stimulation within a treatment period may be implemented in a ramped manner with the display portion 5400 in FIG. 65A schematically representing these implementations. For instance, an automatic start of stimulation may comprise a ramped increase (from zero) to a target stimulation intensity as represented via the triangular shaped ramp symbol shown at 5070. This representation immediately indicates to a viewer the ramped manner in which the stimulation intensity was implemented. The ramped increase may occur at the beginning of a treatment period (e.g. 5405). Similarly, the triangular-shaped ramp symbol 5410 represents a ramped decrease of stimulation intensity from the target level (or another non-zero level) to zero, such as when stimulation is terminated (e.g. at 5406) or when stimulation is to be paused (e.g. at 5080). It will be understood that the representation of a ramped increase or decrease of stimulation intensity may be implemented via shapes other than a triangle.

The gradual ramped initiation or termination of stimulation therapy may enhance a patient's comfort by avoiding abrupt initiation, pause, or cessation of stimulation therapy. Among other features, the ramped implementation may increase the likelihood of patient compliance and appreciation for SDB care.

In some examples, at least some of the features and attributes associated with at least the methods and/or devices represented via FIG. 65A may be implemented via at least some features and attributes of the example methods described hereafter in association with FIGS. 65B-65E. In some examples, the methods described in association with FIGS. 65B-65E may be implemented via devices and elements other than those shown in at least FIG. 65A.

It will be understood that FIG. 65A schematically represents at least some aspects of patient's experience of, operation of a device, and/or a method of treating a patient for sleep apnea. Accordingly, at least some aspects of at least FIG. 65A schematically represent via a method such as example method, as shown at 5500 in FIG. 65B, which comprises automatically taking an action when a probability of sleep, according to the sleep-wake status determination, exceeds a sleep-detection threshold or a probability of wakefulness, according to the sleep-wake status determination, exceeds a wake-detection threshold. In some examples, as shown at 5510 in FIG. 65C, automatically taking an action comprises at least one of automatically starting a stimulation treatment period and automatically stopping the stimulation treatment period. In some such examples, the term “non-sleep” may correspond to a probability of sleep remaining below a sleep detection threshold, while in some such examples, the term “non-wake” may correspond to a probability of wakefulness remaining below a wake detection threshold.

In some such examples (at 5510 in FIG. 65C), an example method may further comprise, as shown at 5520 in FIG. 65D, receiving input to electively start a treatment period and/or to electively stop a treatment period; and upon reception of input to electively start, suspending the automatically start and upon reception of input to electively stop, suspending the automatically terminating.

In some examples, as shown at 5530 in FIG. 65E, a method (associated with the actions/methods in FIGS. 65B-65D) may further comprise tracking information, for a plurality of nightly utilization periods, of at least one of a pattern, trend, and average of at least one of: automatic starts; automatic stops; elective starts; and elective stops. It will be understood that other (or additional) nightly utilization parameters described in association with FIG. 65A may be tracked per method 5530 in FIG. 65E. This tracking information may be used to associate a patient to a cluster of similar patients as described in more detail below with reference to FIG. 69. The associated cluster for a patient may be used to generally characterize the patient and/or to determine eligibility for initiating therapy via automatic sleep detection. Each cluster may be associated with different parameters, criteria, thresholds, etc. for initiating therapy via automatic sleep detection.

FIG. 66 is a block diagram schematically representing an example arrangement 7400 to implement a data model for supporting and/or implementing determination of patient eligibility for automatic sleep detection. The patient eligibility may be based on at least one variability metric of sleep onset latency and/or other parameters. The automatic sleep detection may be used to initiate therapy.

In some examples, the data model may comprise a machine learning element, which may comprise a convolutional neural network, deep neural network, deep neural learning, and the like. It will be understood that in some examples the machine learning element may be implemented via other forms of artificial intelligence tools. The data model may be implemented as part of, or in a complementary manner with, data model parameter 3230 in FIG. 61A.

In some examples, the data model may comprise a heuristic data model or other data model that may be manually tuned. For example, the inputs and outputs of the heuristic data model or other data model may be manually selected and/or the weights applied to each input and/or output may be manually adjusted. In some examples, the heuristic data model or other data model may be manually tuned by a physician, a patient, and/or other person based on observations (e.g. sleep study), feedback (e.g. survey), etc. of and/or from a patient.

In some examples, such a data model arrangement may be used in analyzing sensed physiologic phenomenon (e.g. respiratory signals, cardiac signals, etc.) to determine a pattern(s) indicative of a sleep state (e.g. onset, onset latency, onset latency variability, offset, various sleep stages) and/or pattern(s) indicative of wakefulness (e.g. onset, offset various sleep stages). In some examples, at least part of this analysis may comprise comparing stored signal patterns with current or recent signal patterns.

The output of the data model arrangement 7400 may be provided to, or as, a comprehensive sleep onset determination such as (but not limited to) a comprehensive sleep onset latency variability determination. It will be understood that in some examples, the output of the data model arrangement 7400 may be the sole basis on which a comprehensive sleep onset (e.g. latency variability) determination is implemented. However, in some examples, the output of the data model arrangement 7400 may comprise just one input in a comprehensive sleep onset determination such as (but not limited to) a comprehensive sleep onset latency variability determination. In some examples, the output of the data model may comprise a sleep-wake determination from which the sleep onset determinations may be made.

In some examples, the data model arrangement 7400 as represented in FIG. 66 may comprise a trained data model (e.g. trained deep learning model), which may be trained (i.e. constructed) prior to its operation. As further shown in the example arrangement (e.g. example method or device) in FIG. 44, in some examples the training may be performed at least partially via a resource 7410. In some such examples, the resource 7410 may be external to patient's body and/or external to an implantable medical device 7420. In some examples, the implantable medical device 7420 may comprise an implantable sensor(s) (e.g. accelerometer, impedance, temperature, other) and control portion 4000 (FIG. 62A), among other components, features, etc. In some examples, after such training, the trained data model may be imported into the implantable medical device 7420 for use in determining a sleep-wake state and/or sleep onset latency information (including but not limited to at least one variability metric of sleep onset latency).

In some examples, the resource(s) 7410 (FIG. 66) may comprise a computing resource 7414 sized and scaled to implement a data model such as (but not limited to) performing various forms of machine learning. In some examples, the resource(s) 7410 may comprise a data store 7412, such as (but not limited to) a large data set of stored sleep information for many patients, which may comprise acceleration signal component information, etc. relating to different non-physiologic parameters and physiologic parameters, such as but not limited to cardiac information, respiratory information, motion/activity information, posture information, etc. It will be understood that any one or more of the sensor modalities disclosed within and throughout the present disclosure also contribute to the data store 7412. In some examples, the stored sleep-related data may be specific to the patient in which the trained data model may be imported, such as being imported into or as element within an implantable medical device (e.g. 7420).

With this in mind, in some examples the data model may be trained (i.e. constructed) via the resource 7410 according to the example arrangement (e.g. method and/or device) 7500 in FIG. 67. As is shown in FIG. 67, known inputs 7510 sensed via an accelerometer (e.g. implantable and/or external) and/or other sensing modalities and a known output 7540 are both provided to a trainable data model 7530. In some examples, the known output 7540 may comprise a determined sleep onset such as (but not limited to) variability of sleep onset latency (e.g. such as used to determine patient eligibility for automatic sleep detection to initiate stimulation therapy), which may comprise any number of internally measurable and/or externally measurable physiologic parameters for such determinations. In some such examples, these determinations may comprise parameters used in determining a sleep-wake state, such as but not limited to any one of (or combinations of) EEG, EOG, EMG, ECG, cardiac information, respiratory information, motion/activity, posture, etc.

With this in mind, in some examples the data model may be trained (i.e. constructed) via the resource 7410 according to the example arrangement (e.g. method and/or device) 7500 in FIG. 67. As shown in FIG. 67, known inputs 7510 sensed via an accelerometer (e.g. implantable and/or external) and/or other sensing modalities (e.g. implantable and/or external) and a known output 7540 are both provided to a trainable data model 7530. In some examples, the known output 7540 may comprise a sleep onset latency (which may include latency variability) determination, with the known output based on any number of internally measurable and/or externally measurable physiologic parameters for determining a sleep-wake state, such as but not limited to any one of (or combinations of) EEG, EOG, EMG, ECG, cardiac information, respiratory information, motion/activity, posture, etc.

As further shown in FIG. 67, in some examples at least some known inputs (obtained via the accelerometer and/or other sensor) may comprise any physiologic signal/information, which may comprise cardiac information 7512, respiratory information 7514, motion/activity information 7516, posture information 7518, and/or other information 7519. It will be understood that these inputs are mere examples, and that the known inputs (from the accelerometer signal or other implantable sensors or external sensors) may comprise any sensed physiologic information pertinent to determining a sleep-wake state.

By providing such known inputs 7510 and known outputs 7540 to the trainable data model 7530, a trained data model 7631 (FIG. 46) may be obtained. In some examples, just one or some of the known inputs 7510 may be used, while all of the known inputs 7510 may be used in some examples. As noted elsewhere, in some examples the trainable/trained data model 7530, 7631 may comprise a deep learning model.

FIG. 68 is a diagram schematically representing an example method 7600 (and/or example device) for using a trained data model 7631 for determining sleep onset (e.g. including latency variability in some examples) using internal measurements, such as (but not limited to) via an implanted accelerometer in some examples, and/or other internal or external measurements such as any one or more of the sensing modalities described within and throughout the present disclosure. As shown in FIG. 68, currently sensed inputs 7611 are fed into the trained data model 7631, which then produces a determinable output 7641, such as a current sleep onset determination 7643 (e.g. including latency variability in some examples), which is based on the current inputs 7611. In some examples, the current inputs 7611 correspond to the same type and/or number of known inputs 7510 (FIG. 67) used to train the data model. In some examples, just one or some of the current inputs 7611 may be used, while all of the current inputs 7611 may be used in some examples.

As previously noted, once the trained data model 7631 is obtained, in some examples it is imported into and/or otherwise forms part of control portion 4000 (and/or care engine 2500 in FIG. 61A).

In some examples, other information 7519 (shown in FIGS. 67-68) may comprise input such as from external sensors associated with a remote control 4340, an app 4330 on mobile consumer device 4320, etc. (as shown in FIG. 64 and FIG. 362B) and/or associated with remote, app, physical parameters 3012, 3013, 3018 in FIG. 61A. The external sensors/input may comprise ambient light, movement/operation of the remote control or of the app/mobile consumer device, etc. Other input may comprise time of day, time zone, geographic latitude, etc. as previously described in association with at least FIGS. 53E-53F, temporal parameter 3014 (FIG. 61A), boundary parameter 3016 (FIG. 61A), and the like regarding input used to at least partially determine sleep-wake status according to detecting a probability of sleep and/or a probability of wakefulness.

In some examples, implementing at least some of the example methods and/or devices described in association with FIGS. 1A-68 may comprise use of, and/or determining, at least some of the information provided in FIG. 69. Moreover, the example of FIG. 69 also may comprise an example implementation of at least some of the features of the example methods and/or devices associated with FIGS. 1A-68. With this in mind, FIG. 69 is an example chart 8000 for associating a patient with a cluster of similar patients, i.e. patients which exhibit similar sleep behavior patterns according to various parameters.

As shown in FIG. 69, chart 8000 includes a therapy usage parameter represented on a first radial axis 8002, an amplitude increase parameter represented on a second radial axis 8004, a “late therapy on” parameter represented on a third radial axis 8006, a “therapy on” variation parameter represented on a fourth radial axis 8008, a missed days parameter represented on a fifth radial axis 8010, and a therapy pauses parameter on a sixth radial axis 8012. Each respective radial axis extends from a central origin (A) radially outward to an end point, such as B, C, D, E, F, G for radial axes 8002, 8004, 8006, 8008, 8010, and 8012, respectively. In some examples, each radial axis may represent a range of absolute values from a first value, which may be zero or a selected non-zero value, to a second value of higher magnitude than the first value. However, in some examples, each radial axis may represent a range of relative values from a first relative value, such as “low”, to a second value such as “high”, which is greater than the first relative value. In some examples, at least one of the radial axes may represent values according to a numerical scale (e.g. ones, tens, etc.) which is the same as other radial axes, while in some examples, at least one of the radial axes may represent values according to a numerical scale (e.g. ones, tens, etc.) which is different from other radial axes. The concentric pattern of rings in FIG. 69 represents intervals of different values for the respective radial axes.

As further shown in FIG. 69, chart 8000 includes a first cluster 8020 of similar patients (i.e. patients exhibiting similar sleep patterns), a second cluster 8022 of similar patients, a third cluster 8024 of similar patients, and a fourth cluster 8026 of similar patients. While four clusters relating to six parameters are illustrated in chart 8000, in some examples, more than four clusters may be defined and/or less than six parameters, more than six parameters, or different parameters may be used to define the clusters. As further shown in FIG. 69, each of the different clusters (8020, 8022, 8024, 8026) is represented by a line tracing a path intersecting with the value for each one of the respective different parameters for that group of similar patients.

The associated cluster for a patient may be used to determine eligibility for initiating therapy via automatic sleep detection and/or to adjust parameters, criteria, thresholds, etc. for initiating therapy via automatic sleep detection.

In some examples, therapy usage parameter (axis 8002) indicates how often a patient uses therapy (e.g. average amount of time of therapy usage per day), such as described above with reference to at least FIGS. 65A and 65E. In some examples, amplitude increase parameter (axis 8004) indicate how often, and/or a value of, the amplitude of therapy for a patient is increased (e.g., ramped up), such as described above with reference to at least FIGS. 61A and 65A. In some such examples, the total increase represented along radial axis 8004 may extend from a selectable lower limit (at A) to a selectable upper limit (at C).

In some examples, a “late therapy on” parameter (axis 8006) indicates how late therapy for a patient turns on (e.g., time of day when therapy starts), such as in response to detecting sleep as described above with reference to at least FIG. 41A. In some examples, a “therapy on” variation parameter (axis 8008) indicates the consistency of initiating or turning on of therapy (e.g. time of day when the patient goes to bed), such as variation in the time of elective starts and/or automatic starts described above with reference to at least FIGS. 65D and 65E. In some examples, a missed days parameter (axis 8010) indicates how often a patient misses therapy for an entire day, such as by tracking usage as described above with reference to at least FIGS. 65A and 65E. In some examples, a pauses parameter (axis 8012) indicates how often therapy for a patient is paused, such as elective pauses and/or automatic pauses as described above with reference to at least FIGS. 8, 24, 38A, and 65A.

Accordingly, for the particular example shown in FIG. 69, first cluster 8020 indicates patients with high therapy usage (8002), high amplitude increase (8004), medium “late therapy on” (8006), low “therapy on” variation (8008), low missed days (8010), and low pauses (8012). Patients in the first cluster 8020 use therapy often and increase the amplitude often (within the permitted selectable limits), thus they are the most adherent patients. For patients in this cluster, since there is little variation in when therapy is turned on (e.g. when they go to bed), a consistent time might be weighted more heavily for activating therapy (e.g. patient consistently goes to bed between 11:00 pm and 11:30 pm, therefore that time might be more heavily weighted for detecting sleep). In general terms, a threshold for detecting sleep for this cluster 8020 of patients may be set at a low value.

In some examples, second cluster 8022 indicates patients with medium therapy usage (8002), medium amplitude increase (8004), high “late therapy on” (8006), medium “therapy on” variation (8008), medium missed days (8010), and low pauses (8012). Patients in the second cluster 8022 turn therapy on the latest, but have some variation in when therapy is turned on. For patients in this cluster, detecting sleep may be weighted more heavily toward detecting sleep later, while still being somewhat resistant to detecting sleep onset.

In some examples, third cluster 8024 indicates patients with medium therapy usage (8002), medium amplitude increase (8004), low “late therapy on” (8006), high “therapy on” variation (8008), high missed days (8010), and low pauses (8012). Patients in the third cluster 8024 are highly variable in when therapy is turned on (e.g., when they go to bed), but they have few pauses. For patients in this cluster, an optimal set of parameters might be set to be less sensitive to sleep onset (to avoid accidentally stimulating while they are awake) and less sensitive to wake after sleep onset.

In some examples, fourth cluster 8026 indicates patients with low therapy usage (8002), low amplitude increase (8004), medium “late therapy on” (8006), medium “therapy on” variation (8008), low missed days (8010), and high pauses (8012). Patients in the fourth cluster 8026 do not miss many days of therapy, but when they use the therapy, they do not use the therapy very much and they frequently pause the therapy. However, they are consistent in when therapy is turned on (e.g. when they go to bed). For patients in this cluster, an optimal set of parameters might be set to be more sensitive to (e.g. weighted more heavily for) sleep onset, due to the consistency in when therapy is turned on, but also set to be more sensitive to (e.g. weighted more heavily for) wake after sleep onset, due to the many pauses during the night requiring additional wake detections.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

What is claimed is:

1. A method comprising:
determining patient eligibility, based on at least one variability metric of sleep onset latency of the patient, for initiating therapy via an automatic sleep detection mode.

2. The method of claim 1, further comprising
determining the at least one variability metric via receiving recorded sleep onset latency information for a historical period of nightly sleep treatment periods.

3. The method of claim 1, further comprising determining the at least one variability metric via:
determining sleep onset latency information via:
sensing, via at least one sensor, sleep-related physiologic information for a plurality of nightly sleep treatment periods.

4. The method of claim 3, wherein the at least one sensor includes an accelerometer.

5. The method of claim 3, wherein the sleep onset latency information includes a sleep state of the patient being one of a lying-while-awake state and laying-while-asleep state.

6. The method of claim 3, wherein the sleep-related physiologic information includes at least one of heart rate, temperature, posture, and respiration rate.

7. The method of claim 3, further comprising the step of identifying a quantity of outliers included in the sleep onset latency information, the outliers being a number of standard deviations away from a mean of the sleep onset latency information and overriding the initiation of therapy via the automatic sleep detection mode based on the identified quantity of outliers.

8. The method of claim 7, further comprising initiating automatic sleep detection mode when the quantity of outliers is zero.

9. The method of claim 8, wherein the automatic sleep detection mode includes utilizing a motion-activity sensor to initiate stimulation therapy.

10. The method of claim 7, further comprising initiating automatic sleep detection mode when the quantity of outliers meets a criteria.

11. The method of claim 10, wherein the automatic sleep detection mode includes initiating sleep disordered breathing stimulation therapy after a period of time has elapsed once a posture sensor indicates that the patient posture has not changed for a period of time.

12. The method of claim 10, wherein, when the quantity of outliers is zero, the method including initiating therapy when two modalities indicate a sleep status.

13. The method of claim 1, when criteria is met, automatic sleep detection mode is activated to initiate stimulation therapy.

14. The method of claim 1, delaying stimulation therapy via an automatic sleep detection mode for a set period of time when criteria is met.

15. The method of claim 14, calculating the set period of time from a point in time in which at least one sensor senses that the patient is in a sleep associated position.

16. The method of claim 14, wherein the set period of time is variable based on a historical plurality of nightly sleep treatment periods completed by the patient.

17. The method of claim 1, selecting automatic sleep detection mode from a plurality of modes.

18. The method of claim 17, wherein the plurality of modes include:
a motion-activity only detection mode;
a non-motion-activity physiologic parameter detection mode;
a combination motion-activity and non-motion-activity physiologic parameter detection mode; and
a posture timer detection mode.

19. The method of claim 18, switching from one of the plurality of modes to a second of the plurality of modes.

20. The method of claim 1, wherein the automatic sleep detection mode is solely dependent on motion-activity sensor derived information.

21. The method of claim 1, wherein the automatic sleep detection mode is dependent on accelerometer and non-accelerometer physiologic sensor derived information.

22. The method of claim 1, further comprising re-evaluating the patient eligibility for automatic sleep detection after a selectable number of nightly treatment periods occurring after the step of determining.

23. The method of claim 22, comprising determining the at least one variability metric based on a plurality of parameters, the method further comprising assigning each of the plurality of parameters a weight during the step of re-evaluating.

24. The method of claim 23, wherein the plurality of parameters are selected from the group consisting of: sleep onset latency for one night; mean sleep onset latency; variability in sleep onset; quantity of outliers; severity of outliers, quantity of pauses of the sleep disordered breathing (SDB) stimulation therapy during one night.

25. The method of claim 1, comprising initiating a standard sleep detection mode when the sleep onset latency variability metric is above a threshold.

26. The method of claim 1, comprising initiating the automatic sleep detection mode when the sleep onset latency variability metric is below a threshold.

27. The method of claim 1, further comprising receiving a patient input to initiate therapy.

28. The method of claim 1, comprising implementing the therapy as a stimulation therapy.

29. The method of claim 1, comprising implementing the therapy to treat sleep disordered breathing.

30. The method of claim 29, wherein the therapy comprises a stimulation therapy.

31. The method of claim 1, comprising implementing the stimulation therapy to treat sleep disordered breathing (SDB) to include electrically stimulating at least one upper airway patency-related tissue.

32. The method of claim 1, wherein the at least one variability metric is a measure of time.

33. The method of claim 1, wherein the at least one variability metric is a physical parameter.

34. The method of claim 33, wherein the at least one variability metric includes a plurality of parameters; further wherein the plurality of parameters includes one or more of sleep onset latency for a plurality of nightly sleep treatment periods, mean sleep onset latency for the plurality of nightly sleep treatment periods, variability in sleep onset, number of outliers, and number of user-initiated therapy pauses for each of the plurality of nightly sleep treatment periods.

35. The method of claim 30, wherein the automatic sleep detection mode is one of a plurality of different automatic sleep detection modes.

36. The method of claim 35, wherein in at least one of the plurality of automatic sleep detection modes evaluates changes in at least one of: patient posture, patient motion-activity, and/or non-motion-activity physiologic parameters.

37. The method of claim 35, wherein in at least one of the plurality of automatic sleep detection modes evaluates changes in at least two patient physiologic parameters.

38. The method of claim 1, implementing the automatic sleep detection mode to actuate the therapy after a first set period of time after patient sleep is detected.

39. The method of claim 35, further comprising delaying actuation of the therapy at the end of the first set period of time to a second set period of time when movement of the patient is detected at the end of the first set period of time.

40. The method of claim 39, comprising detecting the movement with an accelerometer.

41. A method for treating sleep disordered breathing, comprising:
applying, via a stimulation element, to an electrical stimulation therapy administered via a stimulation therapy protocol to an upper airway patency-related tissue; and
authorizing initiation of the application of the stimulation therapy protocol for a patient via:
an automatic sleep detection mode upon a value of sleep onset latency variability metric meeting a criteria; and
a standard initiation mode upon the value of the sleep onset latency variability metric not meeting the criteria.

42. The method of claim 41, further comprising:
receiving recorded sleep onset latency information for a historical period of nightly sleep treatment periods.

43. The method of claim 42, further comprising:
determining the sleep onset latency variability metric via:
sensing, via at least one sensor, sleep-related physiologic information for a plurality of nightly sleep treatment periods.

44. The method of claim 41, actuating the stimulation therapy after a set period of time after patient sleep is detected.

45. The method of claim 41, further comprising re-evaluating the stimulation therapy protocol by repeating the steps of claim 35 after the step of authorizing initiation of the application of the stimulation therapy protocol.

46. The method of claim 41, wherein the criteria is defined by the value of sleep onset latency variability metric being below a threshold.

47. The method of claim 41, wherein the criteria is defined by the value of sleep onset latency variability metric being above a threshold.

48. The method of claim 41, further comprising the patient providing an input to indicate a nightly treatment period is about to begin prior to the step of applying electrical stimulation therapy.

49. The method of claim 41, wherein the sleep onset latency variability metric is a measure of time.

50. The method of claim 41, wherein the sleep onset latency variability metric is a physical parameter.

51. The method of claim 41, wherein the automatic sleep detection mode is a selection from a plurality of modes.

52. The method of claim 51, wherein the plurality of modes include:
a motion-activity only detection mode;
a non-motion-activity physiologic parameter detection mode;
a combination motion-activity and non-motion-activity physiologic parameter detection mode; and
a posture timer detection mode.

53. The method of claim 51, further comprising switching from one mode to a second mode.

54. A device comprising:
a control portion programmed to determine patient eligibility of a patient, based on at least one variability metric of sleep onset latency of the patient, for initiating sleep disordered breathing (SDB) stimulation therapy via an automatic sleep detection mode.

55. The device of claim 54, wherein the automatic sleep detection mode is a selection from a plurality of modes.

56. The device of claim 55, wherein the plurality of modes include:
a motion-activity only detection mode;
a non-motion-activity physiologic parameter detection mode;
a combination motion-activity and non-motion-activity physiologic parameter detection mode; and
a posture timer detection mode.
